(19) United States
(12) Patent Application Publication
(10) Pub. No.: US 2010/0062374 A1
(43) Pub. Date: Mar. 11, 2010

(54) POSITIVE RESIST COMPOSITION AND PATTERNING PROCESS

(75) Inventors: Tsunehiro NISHI, Joetsu-shi (JP); Takeshi KINSHO, Joetsu-shi (JP); Masaki OHASHI, Joetsu-shi (JP); Koji HASEGAWA, Joetsu-shi (JP); Masashi IIO, Joetsu-shi (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: SHIN-ETSU CHEMICAL CO., LTD., Tokyo (JP)

(21) Appl. No.: 12/553,441

(22) Filed: Sep. 3, 2009

(30) Foreign Application Priority Data
Sep. 5, 2008 (JP) 2008-227754

(51) Int. Cl. G03F 7/004; G03F 7/20; C07C 69/52

(52) U.S. Cl. 430/286.1; 430/326; 560/220

(57) ABSTRACT

A positive resist composition comprises (A) a resin component which becomes soluble in an alkaline developer under the action of an acid and (B) an acid generator. The resin (A) is a polymer comprising recurring units containing a non-leaving hydroxyl group represented by formula (1) wherein R¹ is H, methyl or trifluoromethyl, X is a single bond or methylene, m is 1 or 2, and the hydroxyl group attaches to a secondary carbon atom. The composition is improved in resolution when processed by lithography.

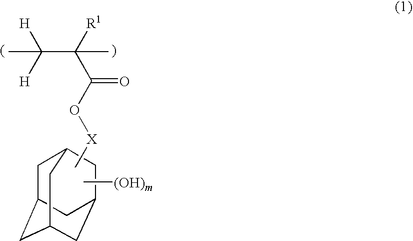

### CROSS-REFERENCE TO RELATED APPLICATION

This non-provisional application claims priority under 35 U.S.C. §119(a) on Patent Application No. 2008-227754 filed in Japan on Sep. 5, 2008, the entire contents of which are hereby incorporated by reference.

### TECHNICAL FIELD

This invention relates to a positive resist composition for the micropatterning technology which is improved in resolution, pattern density dependency and mask fidelity, and a patterning process using the same.

### BACKGROUND ART

In the recent drive for higher integration and operating speeds in LSI devices, it is desired to miniaturize the pattern rule. Great efforts have been devoted for the development of the micropatterning technology using deep-ultraviolet (deep-UV) or vacuum-ultraviolet (VUV) lithography. The photolithography using KrF excimer laser (wavelength 248 nm) as the light source has already established the main role in the commercial manufacture of semiconductor devices. The photolithography using ArF excimer laser (wavelength 193 nm) has commenced commercial manufacture as the advanced microprocessing process. In the ArF excimer laser lithography, a further improvement in resist material performance is strongly demanded to enable a further extension of resolution, partially because the succeeding technology remains uncertain. Efforts have also been made on the development of the immersion lithography designed to extend the resolution by interposing a high refractive index liquid between a resist coating and a projection lens, and a resist material suited for the immersion lithography is thus needed.

The requisite properties for the resist materials adapted for the ArF excimer laser lithography include transparency at wavelength 193 nm and dry etch resistance. Resist materials comprising as a base resin poly(meth)acrylic acid derivatives having bulky acid-labile protective groups as typified by 2-ethyl-2-adamantyl and 2-methyl-2-adamantyl groups were proposed as having both the properties (JP-A 9-73173 and JP-A 9-90637). Since then, a variety of materials have been proposed. Most of them commonly use resins having a highly transparent main chain and a carboxylic acid moiety protected with a bulky tertiary alkyl group.

While the prior art resist materials for the ArF excimer laser lithography suffer from several problems, a decline of resolution by over-diffusion of the acid generated by the photoacid generator is a serious problem. In the ArF excimer laser lithography, the acid generated upon exposure triggers deprotection reaction on the base resin which proceeds during heat treatment following exposure (post-exposure bake or PEB). Movement or diffusion of the acid occurs during PEB. Since chemically amplified resist materials depends on the function of the acid to act as a catalyst to promote deprotection reaction, moderate acid movement is necessary. However, acid movement degrades an optical image, indicating that excess acid movement detracts from resolution. To comply with the outstanding demands for a further size reduction in the ArF excimer laser lithography including and a higher resolution due to a good command of the immersion lithography, there is a need for a resist material featuring controlled acid movement and higher resolution capability.

Citation List

Patent Document 1: JP-A H09-73173

Patent Document 2: JP-A H09-90637

Patent Document 3: U.S. Pat. No. 6,391,520 (JP-A 2000-122295)

### DISCLOSURE OF THE INVENTION

An object of the invention is to provide a positive resist composition which exhibits a high resolution, improved pattern density dependency and mask fidelity when processed by the photolithography using ArF excimer laser light as a light source, and a patterning process using the same.

The inventors have found that a positive resist composition comprising a polymer comprising specific recurring units as a base resin displays an excellent resolution capability when processed by photolithography. The composition is thus quite effective for precise micropatterning.

The invention provides a positive resist composition and a pattern forming process as defined below.

In one aspect, the invention provides a positive resist composition comprising (A) a resin component which becomes soluble in an alkaline developer under the action of an acid and (B) a compound capable of generating an acid in response to actinic light or radiation. The resin component (A) is a polymer comprising recurring units containing a non-leaving hydroxyl group, represented by the general formula (1).

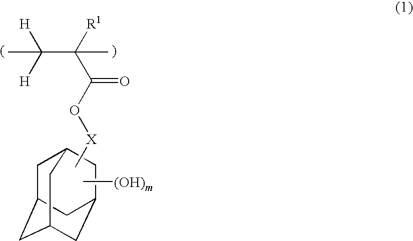

Herein R¹ is hydrogen, methyl or trifluoromethyl, X is a single bond or methylene, and m is 1 or 2. The number “m” of hydroxyl groups each attach to a secondary carbon atom.

In a preferred embodiment, the polymer as resin component (A) further comprises recurring units of the general formulae (2) and (3).

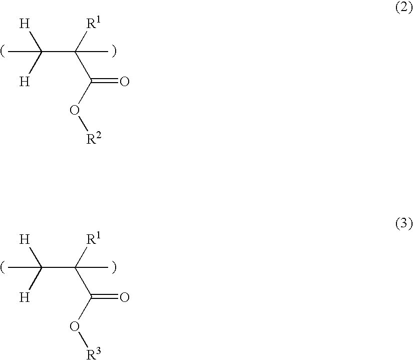

Herein R¹ is each independently hydrogen, methyl or trifluoromethyl, R² is an acid labile group, and R³ is a group containing a 5- or 6-membered lactone ring as a partial structure.

In a preferred embodiment, the compound (B) is a sulfonium salt compound having the general formula (4).

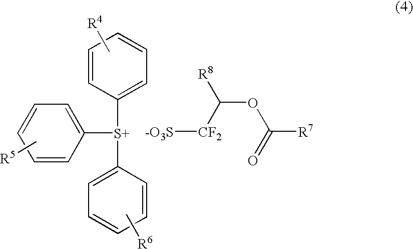

Herein R⁴, R⁵ and R⁶ are each independently hydrogen or a straight, branched or cyclic, monovalent hydrocarbon group of 1 to 20 carbon atoms which may contain a heteroatom, R⁷ is a straight, branched or cyclic, monovalent hydrocarbon group of 7 to 30 carbon atoms which may contain a heteroatom, and R⁸ is hydrogen or trifluoromethyl.

In another aspect, the invention provides a process for forming a pattern, comprising the steps of applying the positive resist composition defined above onto a substrate to form a resist coating; heat treating the resist coating and exposing it to high-energy radiation or electron beam through a photomask; heat treating the exposed coating and developing it with a developer. In one preferred embodiment, the exposing step is effected by the immersion lithography wherein a high refractive index liquid having a refractive index of at least 1.0 intervenes between the resist coating and a projection lens. In another preferred embodiment, the process further comprises the step of applying a protective coating on the resist coating, and the exposing step is effected by the immersion lithography wherein a high refractive index liquid having a refractive index of at least 1.0 intervenes between the protective coating and a projection lens.

Also provided is a polymerizable compound containing a non-leaving hydroxyl group, represented by the general formula (1a):

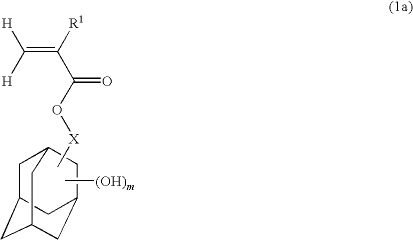

wherein R¹ is hydrogen, methyl or trifluoromethyl, X is a single bond or methylene, and m is 1 or 2, with the proviso that the number “m” of hydroxyl groups each attach to a secondary carbon atom.

### ADVANTAGEOUS EFFECT OF INVENTION

The positive resist composition of the invention exhibits a significantly high resolution when processed by the micropatterning process, especially ArF lithography. The composition is thus quite effective for precise micropatterning.

### DESCRIPTION OF EMBODIMENTS

Below the resist composition of the invention is described in detail. The singular forms “a,” “an” and “the” include plural referents unless the context clearly dictates otherwise. The notation (Cn-Cm) means a group containing from n to m carbon atoms per group.

It is understood that for some structures represented by chemical formulae, there can exist enantiomers and diastereomers because of the presence of asymmetric carbon atoms. In such a case, a single formula collectively represents all such isomers. The isomers may be used alone or in admixture.

The resist composition of the invention comprises (A) a resin component which becomes soluble in an alkaline developer under the action of an acid, and (B) a compound capable of generating an acid in response to actinic light or radiation, wherein resin component (A) is a polymer or high-molecular weight compound comprising recurring units containing a non-leaving hydroxyl group, represented by the general formula (1).

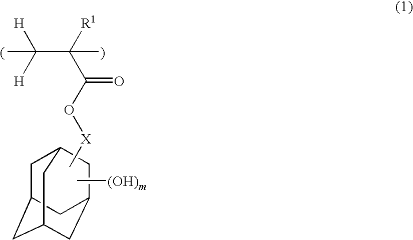

Herein R¹ is hydrogen, methyl or trifluoromethyl, X is a single bond or methylene, and m is 1 or 2, with the proviso that the number “m” of hydroxyl groups each attach to a secondary carbon atom.

As the base resin in resist compositions for the ArF excimer laser lithography, resins having incorporated (meth)acrylate units having a 3-hydroxyadamantan-1-yl or 3,5-dihydroxyadamantan-1-yl group as shown below are widely used.

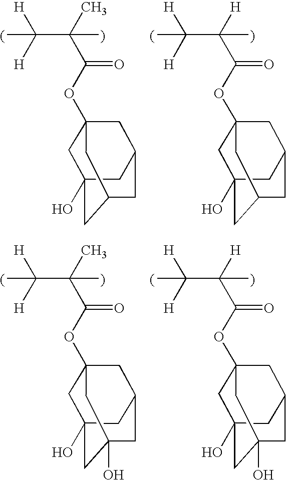

By introducing these recurring units into resins, the movement of the acid generated upon exposure is controlled appropriate so that the resolution capability may be improved. Although the mechanism is not well understood, it is surmised that hydroxyl groups on these units repeat capture and release of proton whereby acid diffusion is controlled. Since these hydroxyl groups are at the bridgehead positions of adamantane ring, they are not eliminated by dehydration reaction and a proton capture ability is never lost. This is also an advantage of the structure.

Based on the hypothetical acid diffusion controlling mechanism of 3-hydroxyadamantan-1-yl (meth)acrylate and 3,5-dihydroxyadamantan-1-yl (meth)acrylate, the inventors tried to design recurring units having a greater acid diffusion controlling effect. It has been found that those units satisfying the following requirements 1) to 4) are compliant with the object.
1) To introduce a non-leaving hydroxyl group for the purpose of imparting a proton capture ability.
2) To introduce a secondary hydroxyl group for the purpose of enhancing the proton capture effect. By virtue of a less influence of intramolecular hydrogen bond as compared with a tertiary hydroxyl group, the secondary hydroxyl group is able to capture proton more effectively.
3) A hydroxyl group may be placed at a position spaced appropriately apart from the main chain via a linking group, for the purpose of enhancing the proton capture effect. The increased distance from the main chain increases the probability of contact between hydroxyl group and proton, with an improvement in proton capture ability being expectable. The introduction of the linking group allows for an appropriate freedom of movement, which also enhances the proton capture effect.
4) To introduce a robust adamantane ring structure in the link between the main chain and the hydroxyl group for the purpose of densifying a resist film to control acid diffusion. This prevents embedment of hydroxyl group in proximity to the main chain and reduces the free volume of a resist film to achieve controlled acid movement.

Most preferred among those units satisfying requirements 1) to 4) are recurring units containing a non-leaving hydroxyl group, represented by formula (1). Exemplary recurring units of formula (1) are illustrated below.

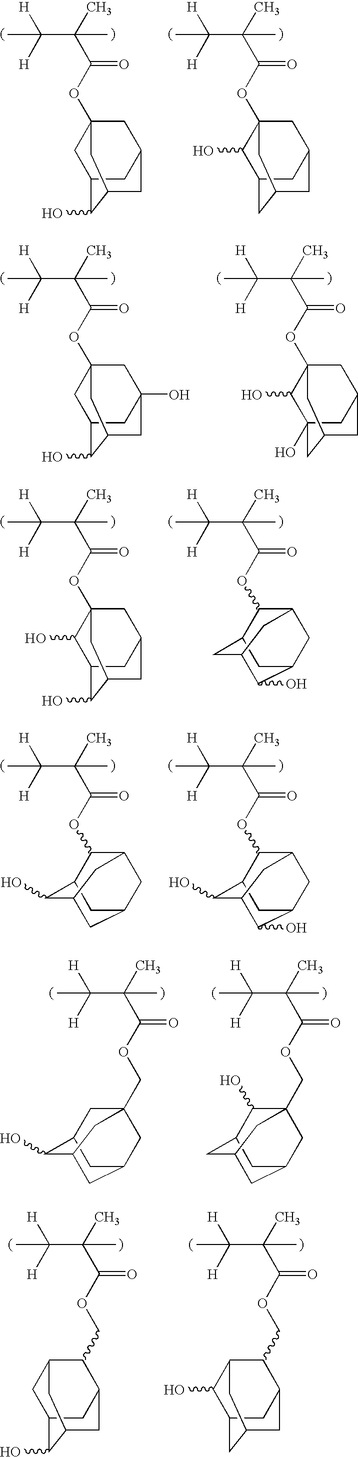

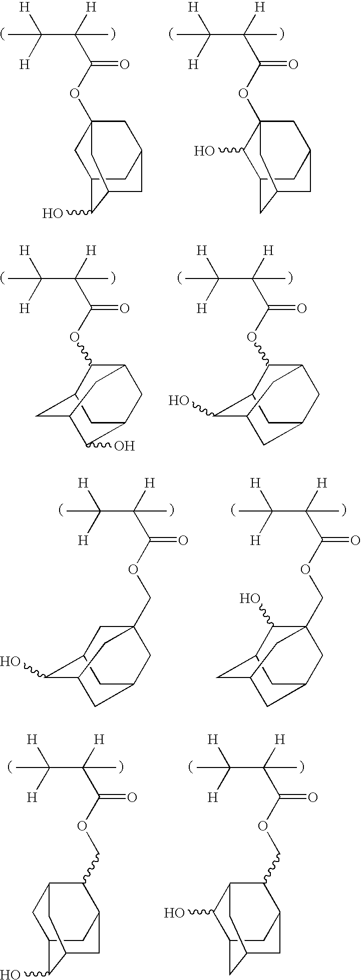

Herein, the wavy line indicates an indefinite direction of the bond. The same applies hereinafter.

In the foregoing examples, the bond position of a hydroxyl group is on a secondary carbon atom of adamantane ring. Since a double bond cannot be formed within the adamantane ring despite the presence of hydrogen atoms on adjacent carbon atoms, elimination of a hydroxyl group by dehydration reaction cannot occur. Now that the above-mentioned recurring units having a secondary hydroxyl group capable of exerting a stronger acid diffusion controlling effect introduced at a non-eliminatable position so that the effect may last long, and optionally a linking group for enhancing the effect are incorporated in a base resin, a resist composition having high resolution, improved pattern density dependency (or optical proximity effect), and mask fidelity may be formulated therefrom.

In the resin component (A) which becomes soluble in an alkaline developer under the action of an acid, the recurring units containing a non-leaving hydroxyl group, represented by formula (1), are introduced in an amount of 1 to 50 mol %, preferably 5 to 40 mol %, and more preferably 10 to 30 mol %, provided that the total of entire recurring units is 100 mol %. Although values outside the range need not be positively excluded, a balance of properties required of the resist material may be disrupted at outside values.

Prior to the present invention, Patent Document 3 (U.S. Pat. No. 6,391,520, JP-A 2000-122295) discloses recurring units possessing an adamantane ring having a non-leaving hydroxyl group introduced therein. In Patent Document 3, hydroxyl and other polar functional groups are introduced for the only purpose of mitigating the hydrophobicity of adamantane ring. By contrast, making investigations on the type and position of a functional group to be introduced and optionally the structure of a linking group for the purpose of improving the acid diffusion controlling effect, the inventors have completed the present invention. While Patent Document 3 intends to introduce a polar functional group at the bridgehead position of adamantane ring as taught in Description of Embodiment and Example sections, it does not encompass the feature of the present invention of introducing a hydroxyl group on secondary carbon of adamantane ring. It is believed that since the present invention has an object and effect different from those of Patent Document 3, employs a construction different from that of Patent Document 3 in a substantial sense, and achieves different results of significance, the present invention is not obvious over Patent Document 3.

In the resist composition of the invention, the resin component (A) which becomes soluble in an alkaline developer under the action of an acid is preferably a polymer further comprising recurring units of the general formulae (2) and (3).

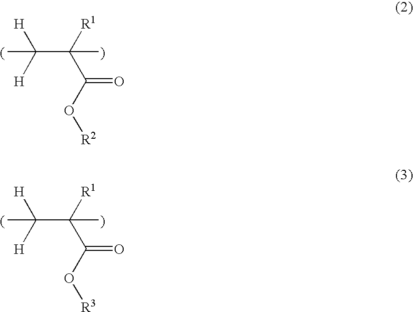

Herein R¹ is each independently hydrogen, methyl or trifluoromethyl, R² is an acid labile group, and R is a group containing a 5- or 6-membered lactone ring as a partial structure.

The acid labile group represented by R² may be selected from a variety of such groups to be deprotected with the acid generated from the photoacid generator to be described later. It may be any of well-known acid labile groups commonly used in prior art resist compositions, especially chemically amplified resist compositions. Examples of the acid labile group are groups of the following general formulae (L1) to (L4), tertiary alkyl groups of 4 to 20 carbon atoms, preferably 4 to 15 carbon atoms, trialkylsilyl groups in which each alkyl moiety has 1 to 6 carbon atoms, and oxoalkyl groups of 4 to 20 carbon atoms.

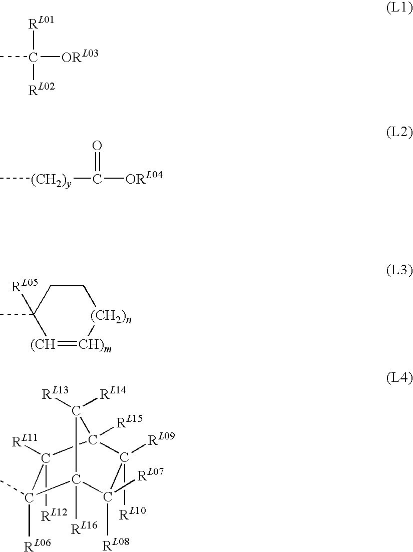

Herein, the broken line denotes a valence bond. In formula (L1), R^{L01} and R^{L02} are hydrogen or straight, branched or cyclic alkyl groups of 1 to 18 carbon atoms, preferably 1 to 10 carbon atoms. Exemplary alkyl groups include methyl, ethyl, propyl, isopropyl, n-butyl, sec-butyl, tert-butyl, cyclopentyl, cyclohexyl, 2-ethylhexyl, n-octyl, and adamantyl. R^{L03} is a monovalent hydrocarbon group of 1 to 18 carbon atoms, preferably 1 to 10 carbon atoms, which may contain a heteroatom such as oxygen, examples of which include unsubstituted straight, branched or cyclic alkyl groups and substituted forms of such alkyl groups in which some hydrogen atoms are replaced by hydroxyl, alkoxy, oxo, amino, alkylamino or the like. Illustrative examples of the straight, branched or cyclic alkyl groups are as exemplified above for R^{L01} and R^{L02}, and examples of the substituted alkyl groups are as shown below.

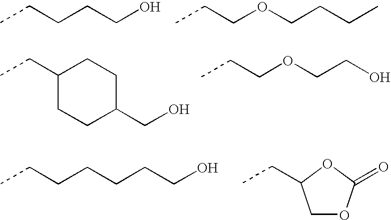

A pair of R^{L01} and R^{L02}, R^{L01}, and R^{L03}, or R^{L02} and R^{L03} may bond together to form a ring with carbon and oxygen atoms to which they are attached. Each of R^{L01}, R^{L02} and R^{L03} is a straight or branched alkylene group of 1 to 18 carbon atoms, preferably 1 to 10 carbon atoms when they form a ring.

In formula (L2), R^{L04} is a tertiary alkyl group of 4 to 20 carbon atoms, preferably 4 to 15 carbon atoms, a trialkylsilyl group in which each alkyl moiety has 1 to 6 carbon atoms, an oxoalkyl group of 4 to 20 carbon atoms, or a group of formula (L1). Exemplary tertiary alkyl groups are tert-butyl, tert-amyl, 1,1-diethylpropyl, 2-cyclopentylpropan-2-yl, 2-cyclohexylpropan-2-yl, 2-(bicyclo[2.2.1]heptan-2-yl)propan-2-yl, 2-(adamantan-1-yl)propan-2-yl, 2-(tricyclo[5.2.1.0^{2,6}]decan-8-yl)propan-2-yl, 2-(tetracyclo[4.4.0.1^{2,5}.1^{7,10}]dodecan-3-yl)propan-2-yl, 1-ethylcyclopentyl, 1-butylcyclopentyl, 1-ethylcyclohexyl, 1-butylcyclohexyl, 1-ethyl-2-cyclopentenyl, 1-ethyl-2-cyclohexenyl, 2-methyl-2-adamantyl, 2-ethyl-2-adamantyl, 8-methyl-8-tricyclo[5.2.1.0^{2,6}]decyl, 8-ethyl-8-tricyclo[5.2.1.0^{2,6}]decyl, 3-methyl-3-tetracyclo[4.4.0.1^{2,5}.1^{7,10}]dodecyl, 3-ethyl-3-tetracyclo[4.4.0.1^{2,5}.1^{7,10}]dodecyl, and the like. Exemplary trialkylsilyl groups are trimethylsilyl, triethylsilyl, and dimethyl-tert-butylsilyl. Exemplary oxoalkyl groups are 3-oxocyclohexyl, 4-methyl-2-oxooxan-4-yl, and 5-methyl-2-oxooxolan-5-yl. Letter y is an integer of 0 to 6.

In formula (L3), R^{L05} is an optionally substituted, straight, branched or cyclic C₁-C₁₀ alkyl group or an optionally substituted C₆-C₂₀ aryl group. Examples of the optionally substituted alkyl groups include straight, branched or cyclic alkyl groups such as methyl, ethyl, propyl, isopropyl, n-butyl, sec-butyl, tert-butyl, tert-amyl, n-pentyl, n-hexyl, cyclopentyl, cyclohexyl, and bicyclo[2.2.1]heptyl, and substituted forms of such groups in which some hydrogen atoms are replaced by hydroxyl, alkoxy, carboxy, alkoxycarbonyl, oxo, amino, alkylamino, cyano, mercapto, alkylthio, sulfo or other groups or in which some methylene groups are replaced by oxygen or sulfur atoms. Examples of optionally substituted aryl groups include phenyl, methylphenyl, naphthyl, anthryl, phenanthryl, and pyrenyl. Letter m is equal to 0 or 1, n is equal to 0, 1, 2 or 3, and 2m+n is equal to 2 or 3.

In formula (L4), R^{L06} is an optionally substituted, straight, branched or cyclic C₁-C₁₀ alkyl group or an optionally substituted C₆-C₂₀ aryl group. Examples of these groups are the same as exemplified for R^{L05}. R^{L07} to R^{L16} independently represent hydrogen or monovalent hydrocarbon groups of 1 to 15 carbon atoms. Exemplary hydrocarbon groups are straight, branched or cyclic alkyl groups such as methyl, ethyl, propyl, isopropyl, n-butyl, sec-butyl, tert-butyl, tert-amyl, n-pentyl, n-hexyl, n-octyl, n-nonyl, n-decyl, cyclopentyl, cyclohexyl, cyclopentylmethyl, cyclopentylethyl, cyclopentylbutyl, cyclohexylmethyl, cyclohexylethyl and cyclohexylbutyl, and substituted forms of these groups in which some hydrogen atoms are replaced by hydroxyl, alkoxy, carboxy, alkoxycarbonyl, oxo, amino, alkylamino, cyano, mercapto, alkylthio, sulfo or other groups. Alternatively, two of R^{L07} to R^{L16} may bond together to form a ring with the carbon atom(s) to which they are attached (for example, a pair of R^{L07} and R^{L08}, R^{L07} and R^{L09}, R^{L08} and R^{L10}, R^{L09} and R^{L10}, R^{L11} and R^{L12}, R^{L13} and R^{L14}, or a similar pair form a ring). Each of R^{L07} to R^{L16} represents a divalent C₁-C₁₅ hydrocarbon group when they form a ring, examples of which are those exemplified above for the monovalent hydrocarbon groups, with one hydrogen atom being eliminated. Two of R^{L07} to R^{L16} which are attached to vicinal carbon atoms may bond together directly to form a double bond (for example, a pair of R^{L07} and R^{L09}, R^{L09} and R^{L15}, R^{L13} and R^{L15}, or a similar pair).

Of the acid labile groups of formula (L1), the straight and branched ones are exemplified by the following groups.

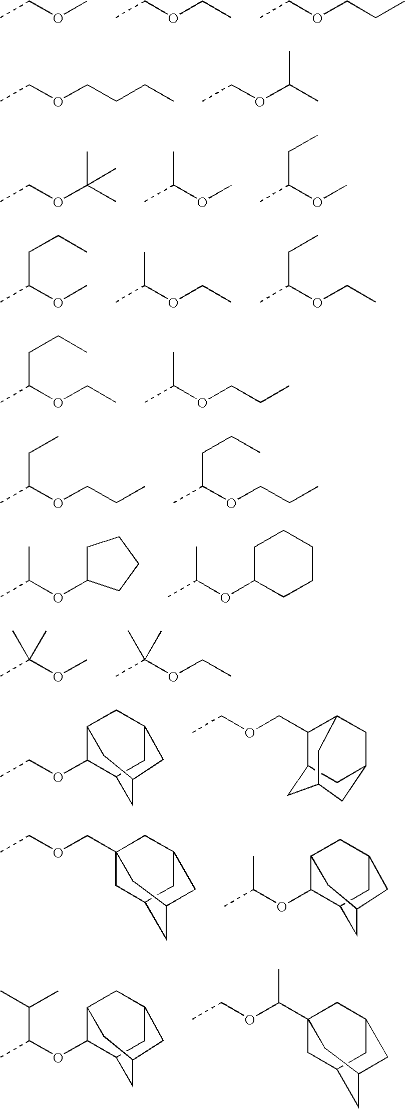

Of the acid labile groups of formula (L1), the cyclic ones are, for example, tetrahydrofuran-2-yl, 2-methyltetrahydrofuran-2-yl, tetrahydropyran-2-yl, and 2-methyltetrahydropyran-2-yl.

Examples of the acid labile groups of formula (L2) include tert-butoxycarbonyl, tert-butoxycarbonylmethyl, tert-amyloxycarbonyl, tert-amyloxycarbonylmethyl, 1,1-diethylpropyloxycarbonyl, 1,1-diethylpropyloxycarbonylmethyl, 1-ethylcyclopentyloxycarbonyl, 1-ethylcyclopentyloxycarbonylmethyl, 1-ethyl-2-cyclopentenyloxycarbonyl, 1-ethyl-2-cyclopentenyloxycarbonylmethyl, 1-ethoxyethoxycarbonylmethyl, 2-tetrahydropyranyloxycarbonylmethyl, and 2-tetrahydrofuranyloxycarbonylmethyl groups.

Examples of the acid labile groups of formula (L3) include 1-methylcyclopentyl, 1-ethylcyclopentyl, 1-n-propylcyclopentyl, 1-isopropylcyclopentyl, 1-n-butylcyclopentyl, 1-sec-butylcyclopentyl, 1-cyclohexylcyclopentyl, 1-(4-methoxybutyl)cyclopentyl, 1-(bicyclo[2.2.1]heptan-2-yl)cyclopentyl, 1-(7-oxabicyclo[2.2.1]heptan-2-yl)cyclopentyl, 1-methylcyclohexyl, 1-ethylcyclohexyl, 1-methyl-2-cyclopentenyl, 1-ethyl-2-cyclopentenyl, 1-methyl-2-cyclohexenyl, and 1-ethyl-2-cyclohexenyl groups.

Of the acid labile groups of formula (L4), those groups of the following formulae (L4-1) to (L4-4) are preferred.

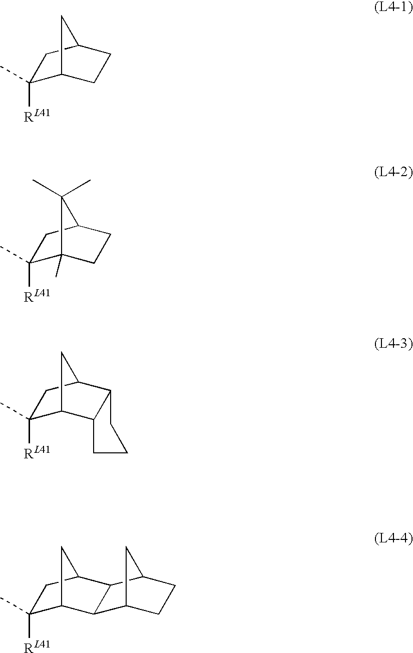

In formulas (L4-1) to (L4-4), the broken line denotes a bonding site and direction. R^{L41} is each independently a monovalent hydrocarbon group, typically a straight, branched or cyclic C₁-C₁₀ alkyl group, such as methyl, ethyl, propyl, isopropyl, n-butyl, sec-butyl, tert-butyl, tert-amyl, n-pentyl, n-hexyl, cyclopentyl and cyclohexyl.

For formulas (L4-1) to (L4-4), there can exist enantiomers and diastereomers. Each of formulae (L4-1) to (L4-4) collectively represents all such stereoisomers. Such stereoisomers may be used alone or in admixture.

For example, the general formula (L4-3) represents one or a mixture of two selected from groups having the following general formulas (L4-3-1) and (L4-3-2).

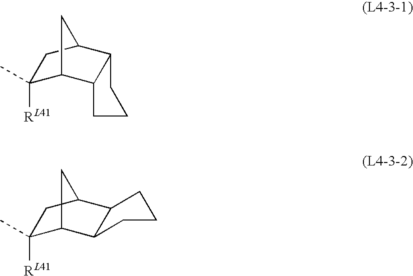

Similarly, the general formula (L4-4) represents one or a mixture of two or more selected from groups having the following general formulas (L4-4-1) to (L4-4-4).

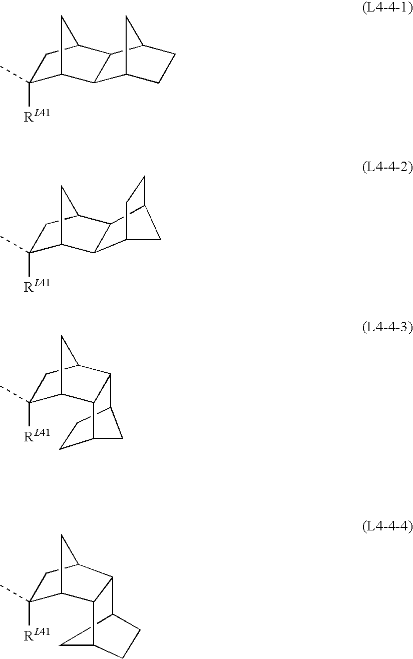

Each of formulas (L4-1) to (L4-4), (L4-3-1) and (L4-3-2), and (L4-4-1) to (L4-4-4) collectively represents an enantiomer thereof and a mixture of enantiomers.

It is noted that in the above formulas (L4-1) to (L4-4), (L4-3-1) and (L4-3-2), and (L4-4-1) to (L4-4-4), the bond direction is on the exo side relative to the bicyclo[2.2.1]heptane ring, which ensures high reactivity for acid catalyzed elimination reaction (see JP-A 2000-336121). In preparing these monomers having a tertiary exo-alkyl group of bicyclo[2.2.1]heptane structure as a substituent group, there may be contained monomers substituted with an endo-alkyl group as represented by the following formulas (L4-1-endo) to (L4-4-endo). For good reactivity, an exo proportion of at least 50 mol % is preferred, with an exo proportion of at least 80 mol % being more preferred.

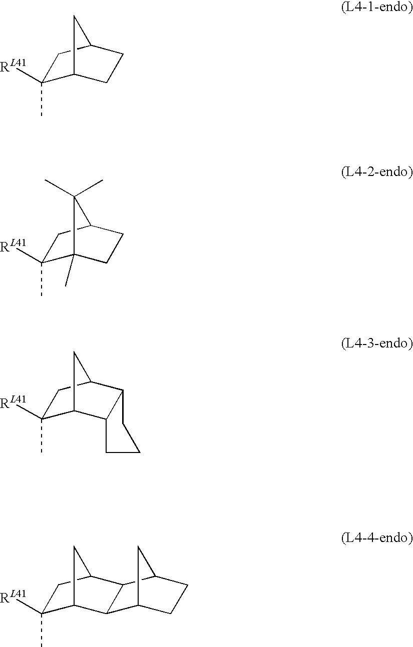

Illustrative examples of the acid labile group of formula (L4) are given below.

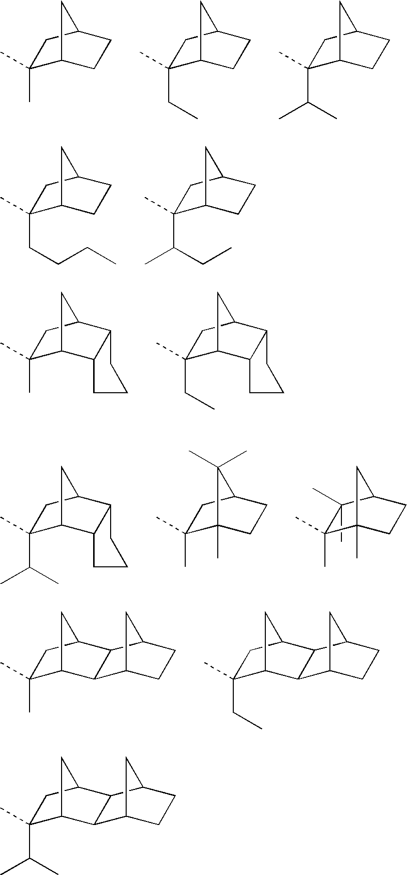

Examples of the tertiary C₄-C₂₀ alkyl groups, trialkylsilyl groups in which each alkyl moiety has 1 to 6 carbon atoms, and C₄-C₂₀ oxoalkyl groups are as exemplified for R^{L04} and the like.

In the resin component (A) which becomes soluble in an alkaline developer under the action of an acid, the recurring units having an acid labile group, represented by formula (2), are introduced in an amount of 5 to 80 mol %, preferably 10 to 70 mol %, and more preferably 15 to 65 mol % provided that the total of entire recurring units is 100 mol %. Although values outside the range need not be positively excluded, a balance of properties required of the resist material may be disrupted at outside values.

R³ is a group containing a 5- or 6-membered lactone ring as a partial structure, examples of which are illustrated below, but not limited thereto.

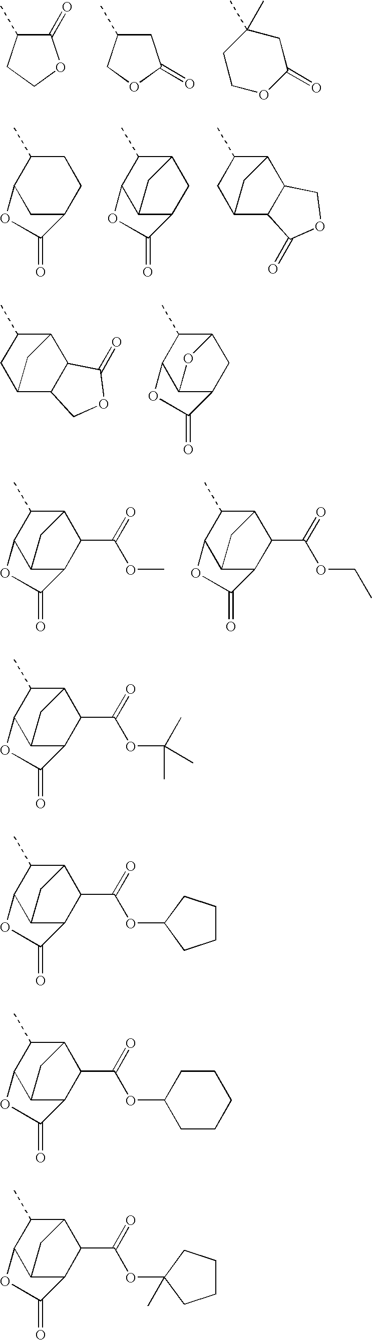

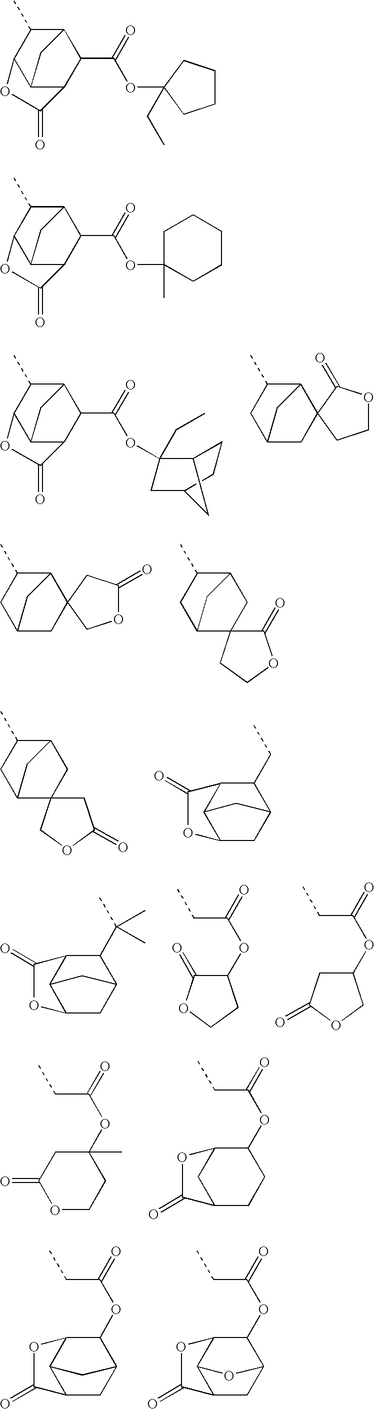

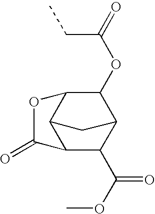

In the resin component (A) which becomes soluble in an alkaline developer under the action of an acid, the recurring units having a 5- or 6-membered lactone ring, represented by formula (3), are introduced in an amount of 5 to 80 mol %, preferably 10 to 70 mol %, and more preferably 15 to 65 mol %, provided that the total of entire recurring units is 100 mol %. Although values outside the range need not be positively excluded, a balance of properties required of the resist material may be disrupted at outside values.

In addition to the non-leaving hydroxyl group-containing recurring units of formula (1), the acid labile group-containing recurring units of formula (2), and the 5- or 6-membered lactone ring-containing recurring units of formula (3), the resin component (A) which becomes soluble in an alkaline developer under the action of an acid may further comprise additional recurring units, as shown below, in an amount of 0 to 50 mol %, and preferably 0 to 40 mol %, provided that the total of entire recurring units is 100 mol %.

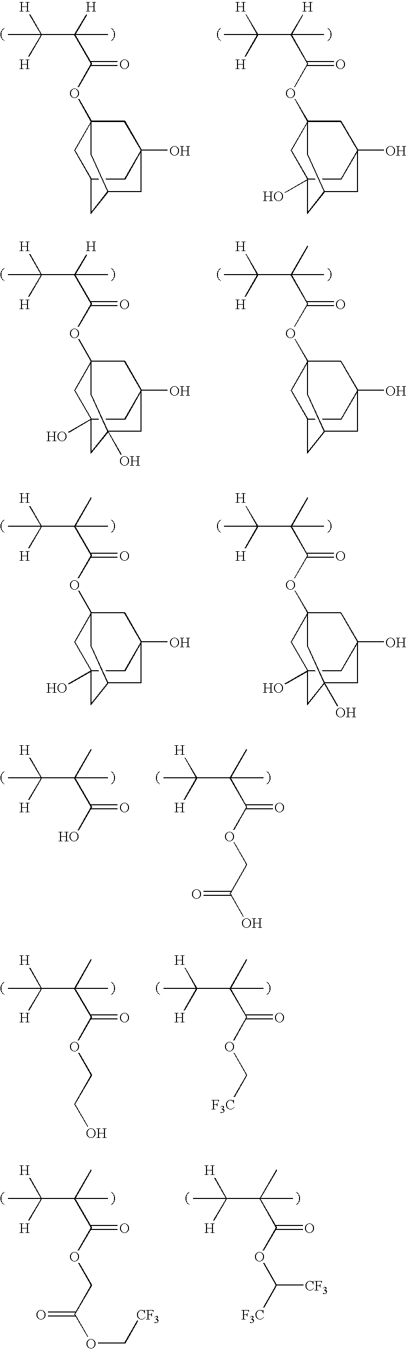

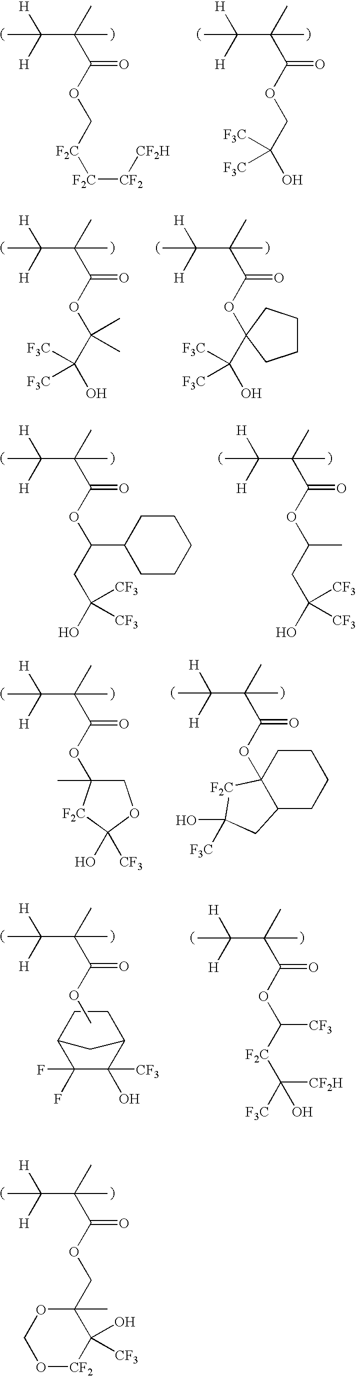

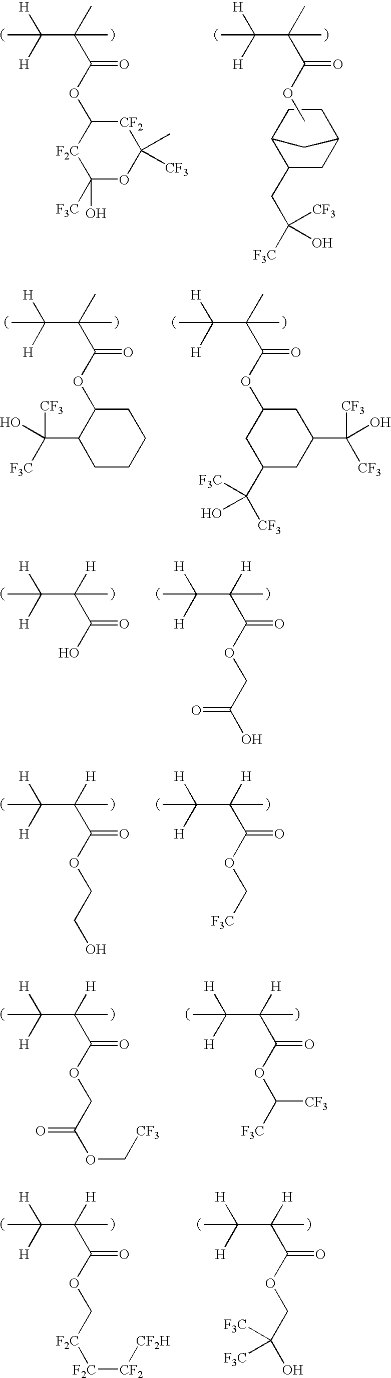

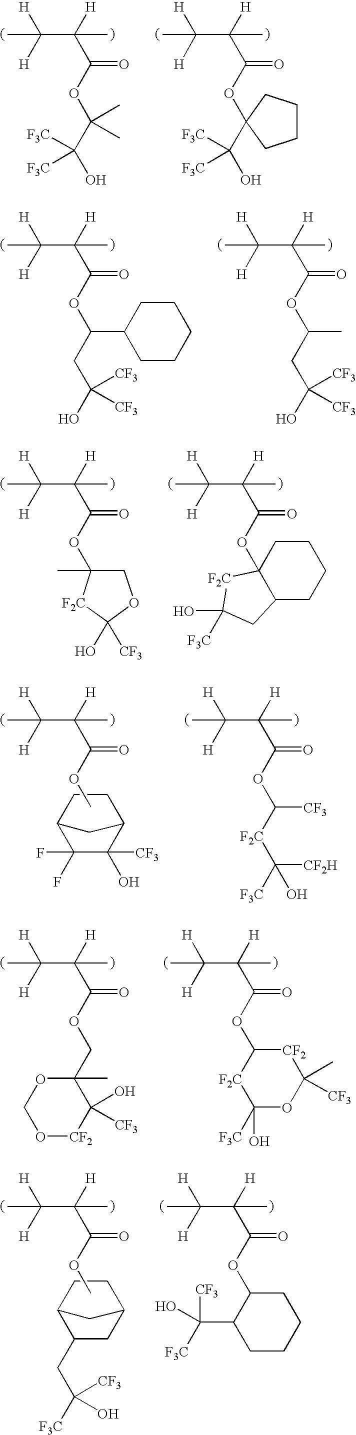

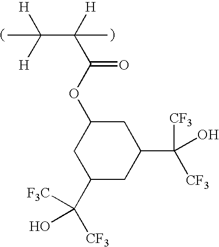

Exemplary preferred constructions of resin component (A) are given below, but not limited thereto.

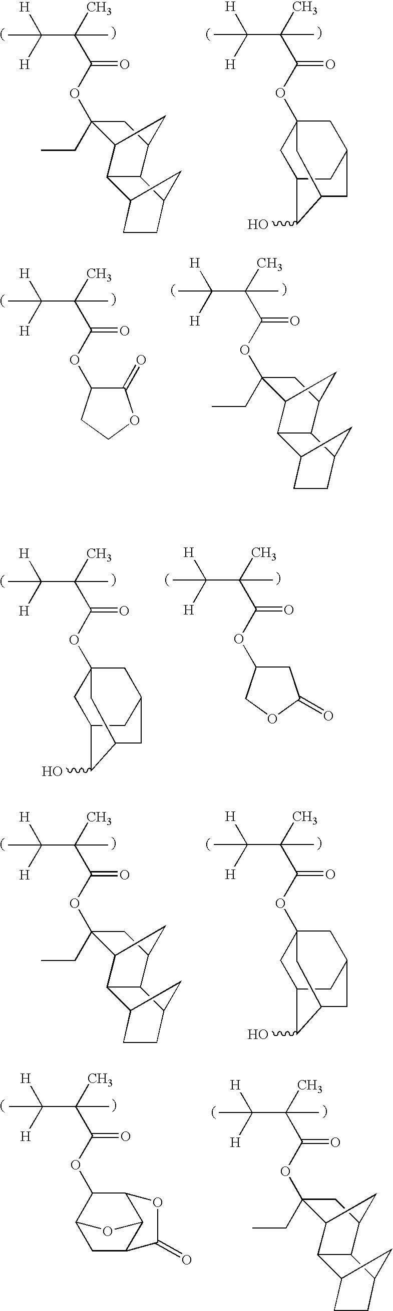

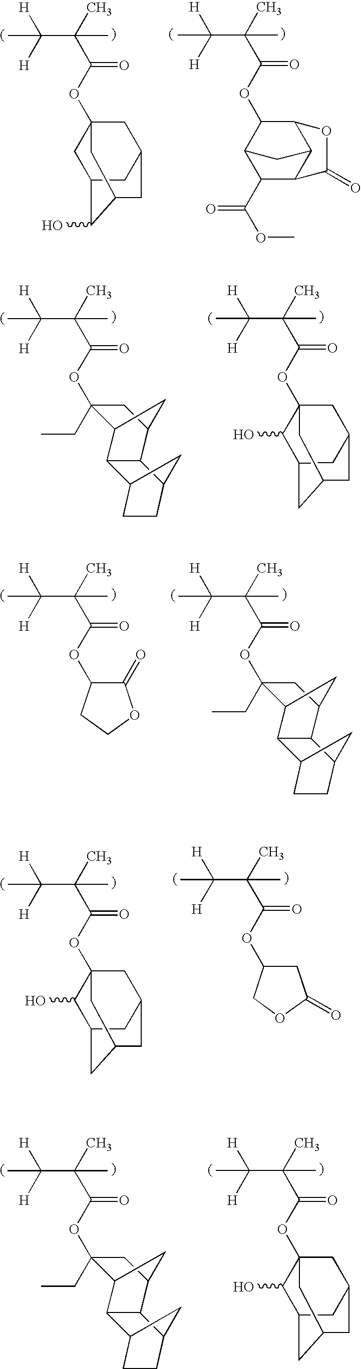

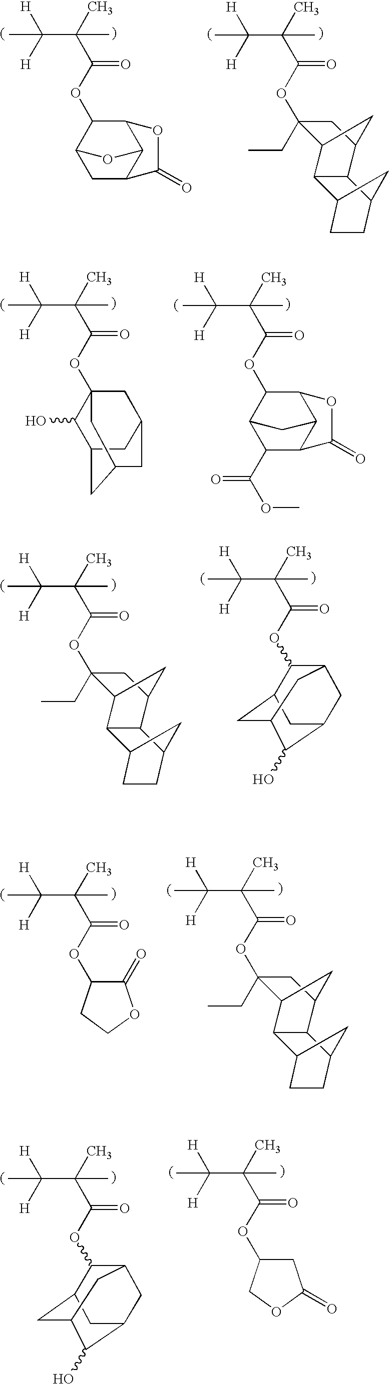

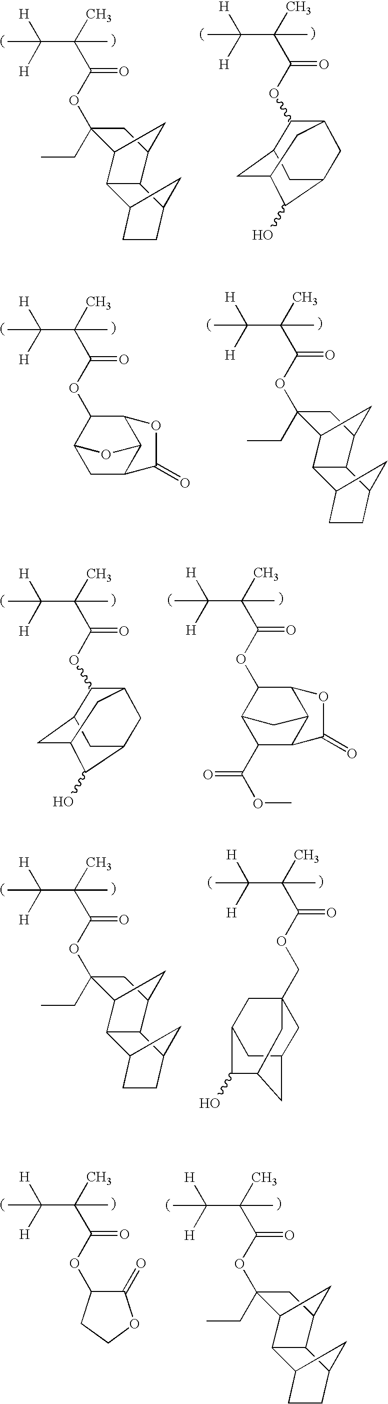

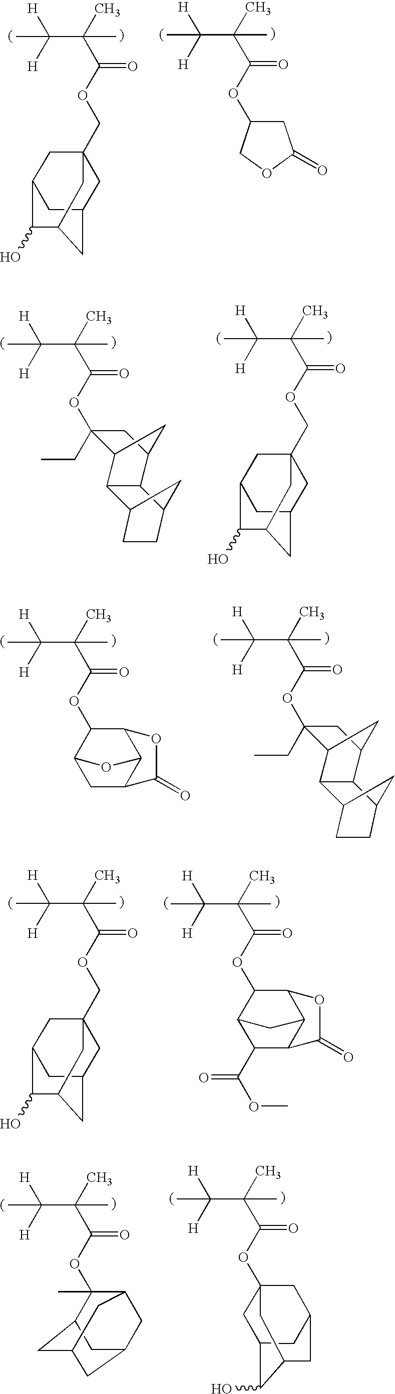

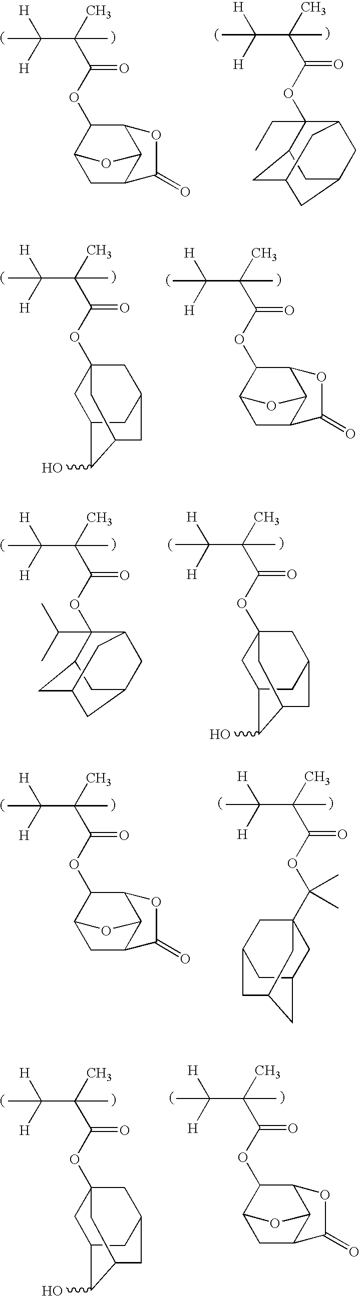

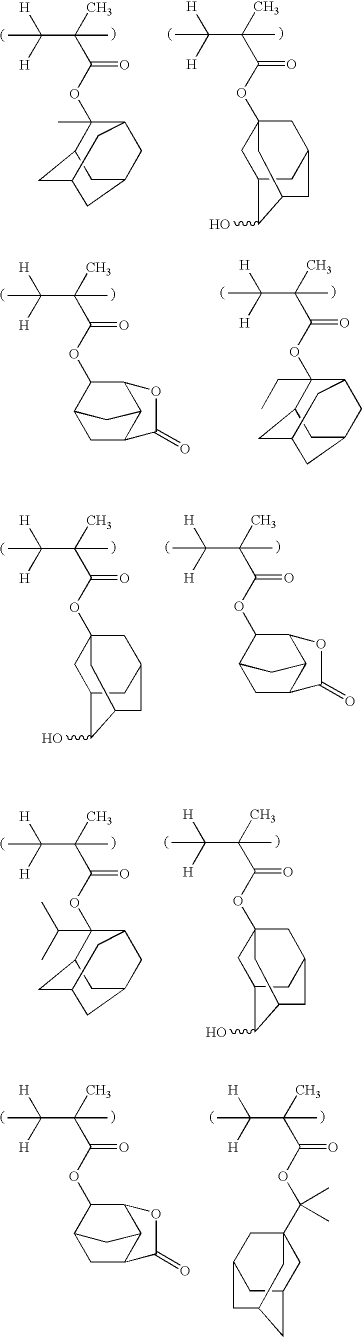

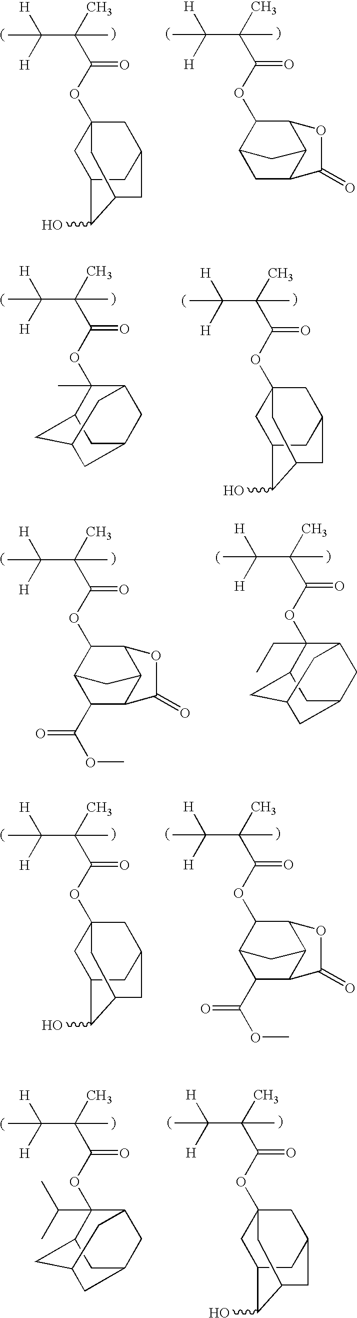

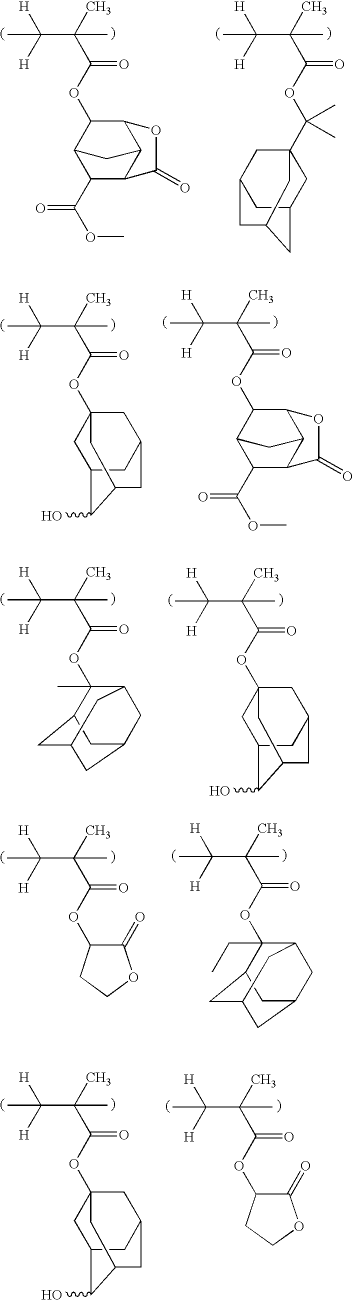

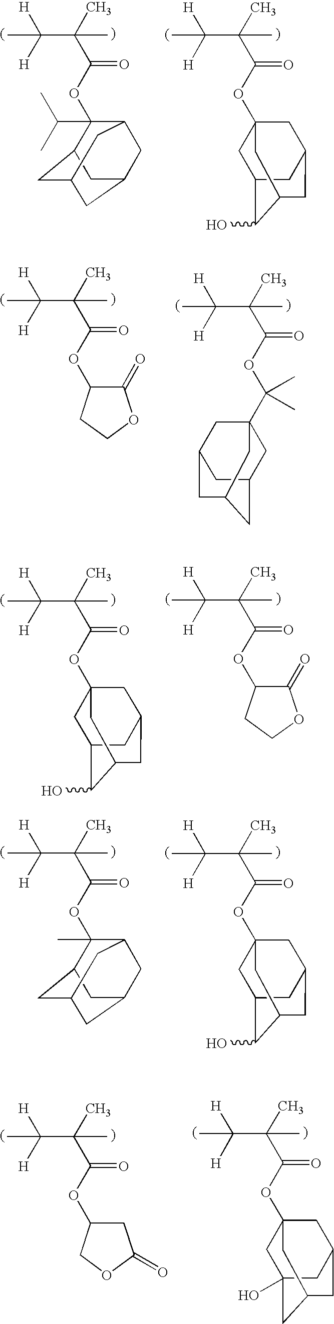

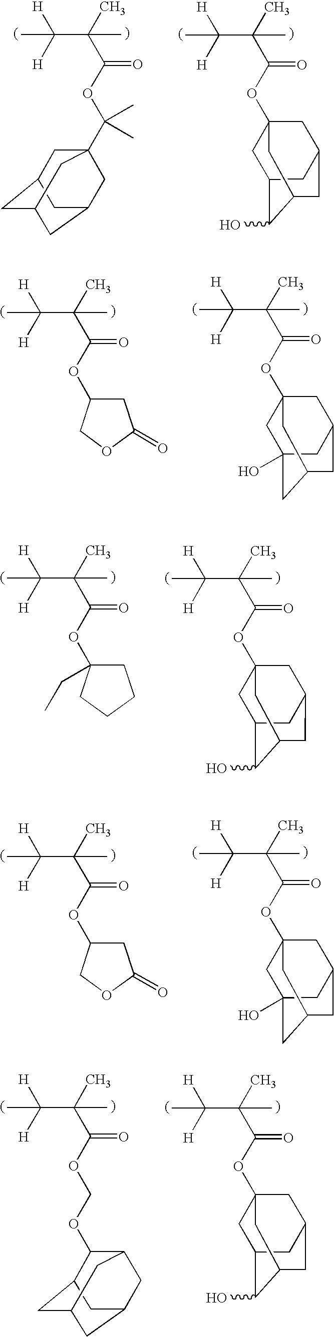

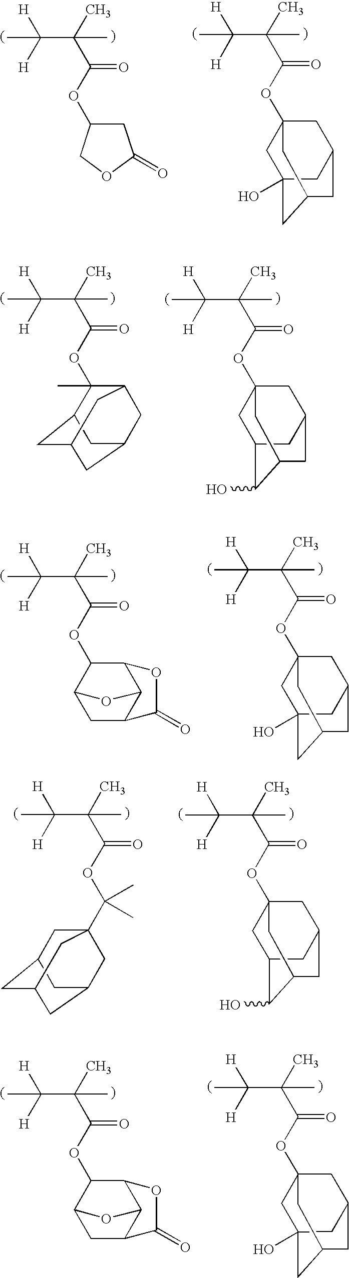

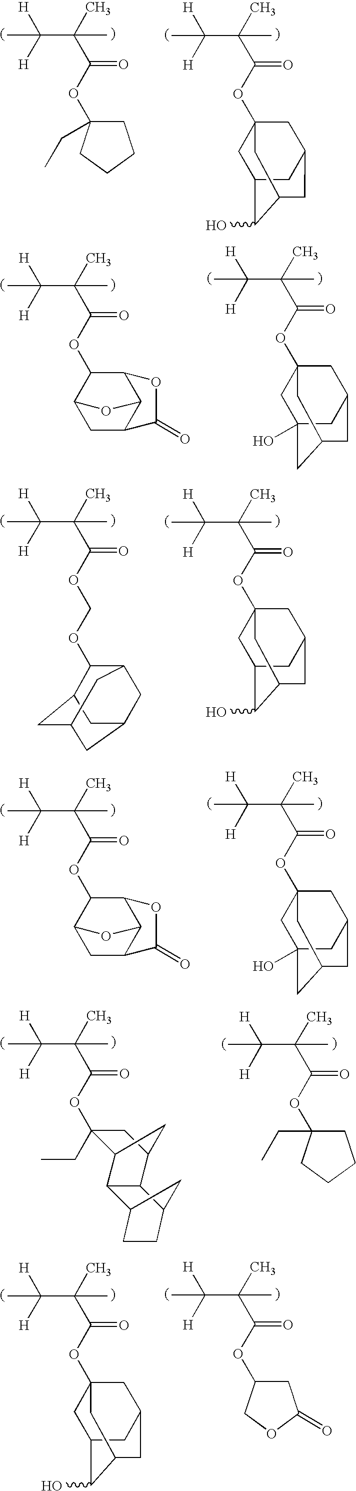

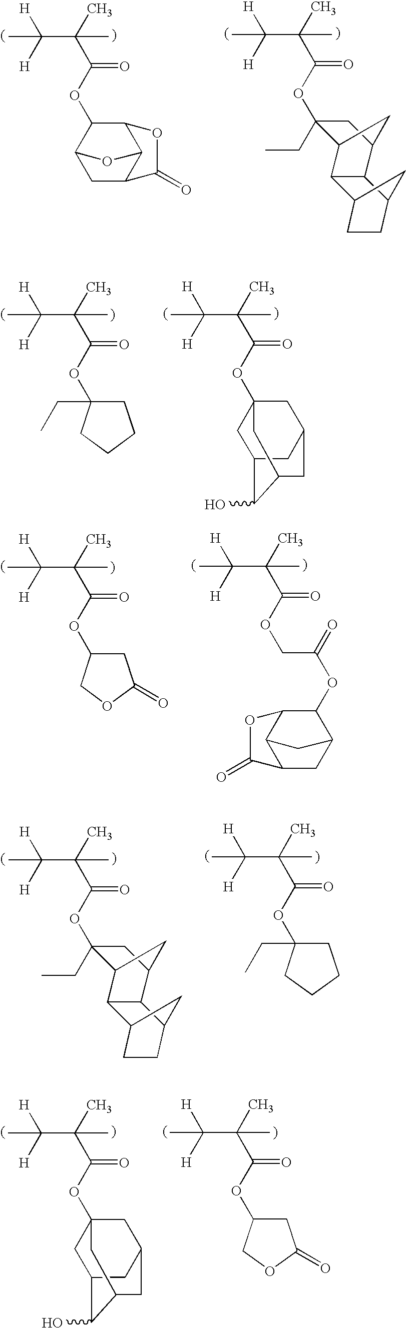

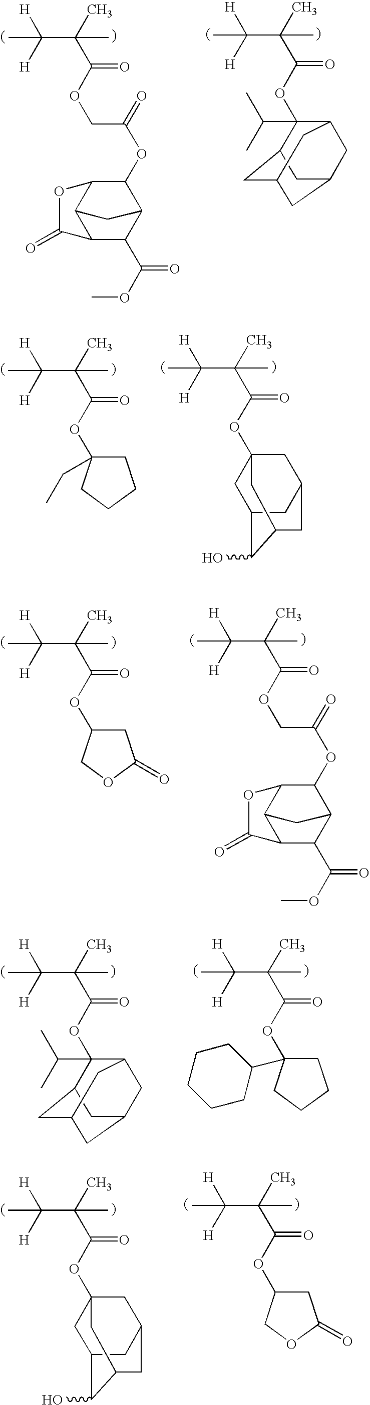

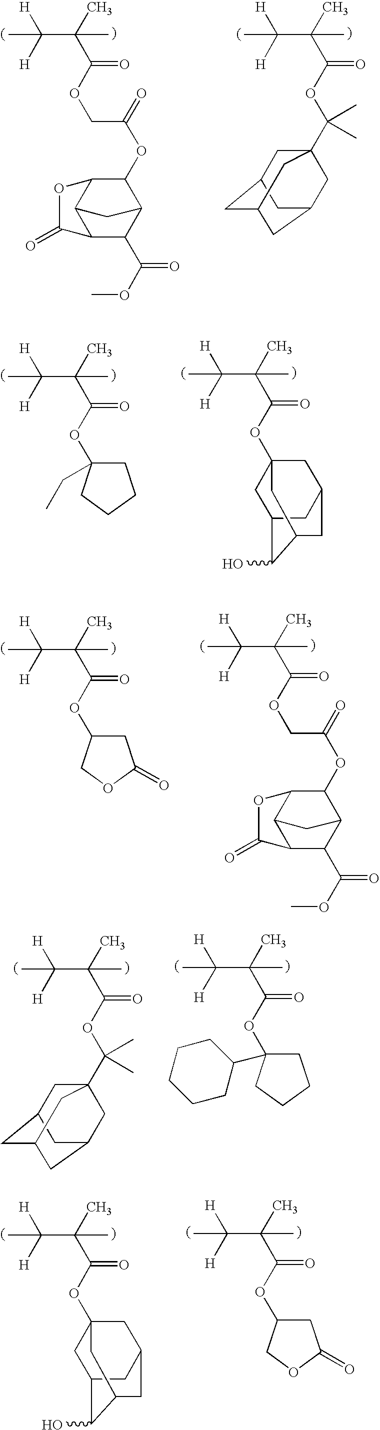

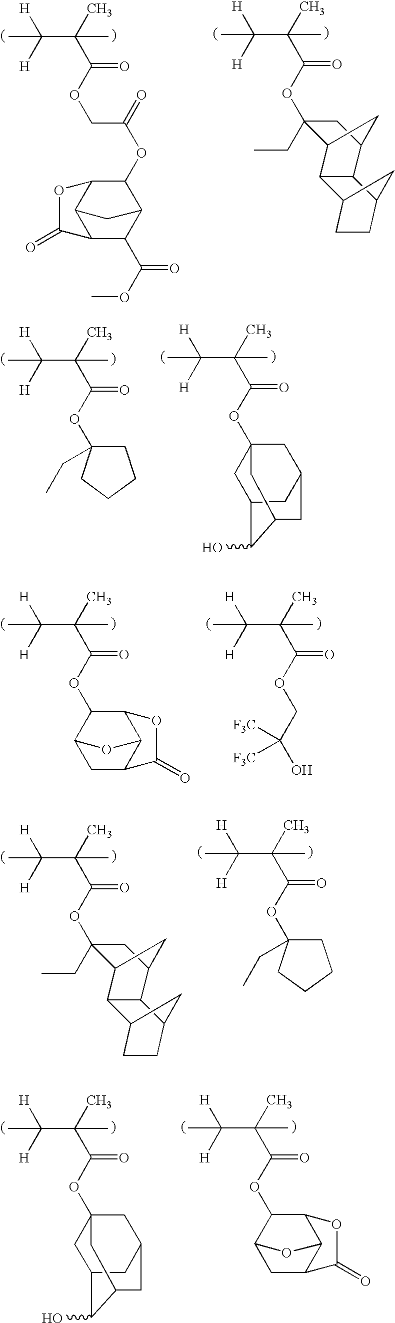

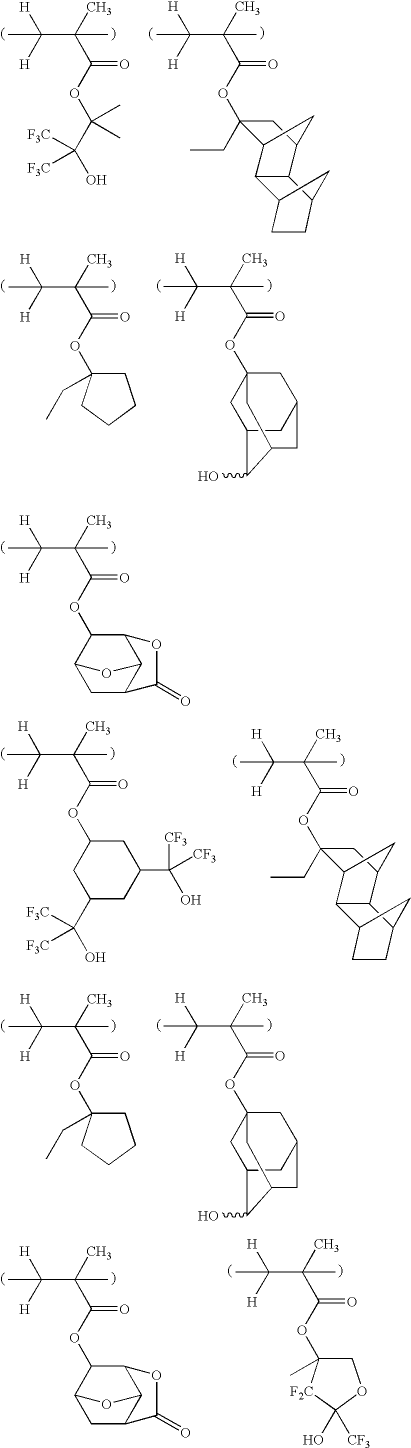

The polymer as resin component (A) should preferably have a weight average molecular weight (Mw) of 1,000 to 50,000, and more preferably 2,000 to 30,000, as measured by gel permeation chromatography (GPC) versus polystyrene standards.

The polymer as resin component (A) may be obtained through copolymerization of (meth)acrylate derivative monomers corresponding to the respective recurring units by any well-known technique such as radical polymerization. It is noted that the polymers used in Examples to be described later were synthesized from preselected (meth)acrylate derivative monomers by a standard radical polymerization technique.

The recurring units of formula (1) are derived from a polymerizable compound containing a non-leaving hydroxyl group, represented by the general formula (1a):

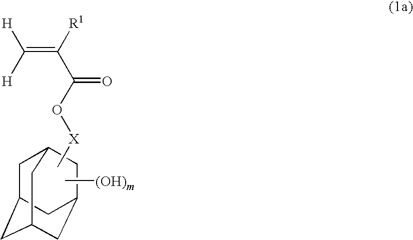

wherein R¹ is hydrogen, methyl or trifluoromethyl, X is a single bond or methylene, and m is 1 or 2, with the proviso that the number “m”, of hydroxyl groups each attach to a secondary carbon atom.

The compound of formula (1a) may be prepared by starting with a commercially available hydroxyadamantanone resulting from oxidation of 1-adamantanol or 1,3-adamantanediol, and treating it with (meth)acrylic anhydride or an acid halide such as (meth)acrylic acid chloride in the presence of a base such as triethylamine or pyridine, to form a ketone-containing ester compound. This compound is treated with sodium borohydride to selectively reduce only the ketone moiety, obtaining the desired compound of formula (1a).

In a preferred embodiment, the compound (B) capable of generating an acid in response to actinic light or radiation is a sulfonium salt compound having the general formula (4):

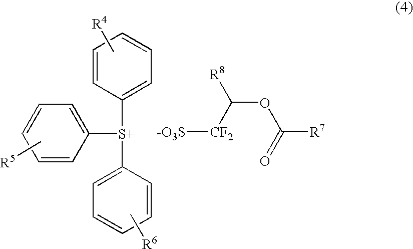

wherein R⁴, R⁵ and R⁶ are each independently hydrogen or a straight, branched or cyclic, monovalent hydrocarbon group of 1 to 20 carbon atoms which may contain a heteroatom, R⁷ is a straight, branched or cyclic, monovalent hydrocarbon group of 7 to 30 carbon atoms which may contain a heteroatom, and R⁸ is hydrogen or trifluoromethyl.

Use of a sulfonium salt having formula (4) as an acid generator is effective in controlling acid diffusion and improving resolution capability. Upon exposure, the compound of formula (4) generates a fluoroalkanesulfonic acid, whose mobility is substantially restrained due to a bulky partial structure and a polar group, as compared with simple perfluoroalkanesulfonic acids such as nonafluorobutanesulfonic acid. Accordingly, a polymer comprising non-leaving hydroxyl group-containing recurring units of formula (1) may be used, preferably in combination with an acid generator of formula (4), to formulate a resist composition which is characterized by effectively controlled acid diffusion and capable of forming a pattern faithful to an optical image.

Below the compound (B) capable of generating an acid in response to actinic light or radiation is further illustrated. In formula (4), R⁴, R⁵ and R⁶ are each independently hydrogen or a straight, branched or cyclic, monovalent hydrocarbon group of 1 to 20 carbon atoms which may contain a heteroatom(s). Exemplary hydrocarbon groups include methyl, ethyl, propyl, isopropyl, n-butyl, sec-butyl, tert-butyl, tert-amyl, n-pentyl, n-hexyl, cyclopentyl, cyclohexyl, ethylcyclopentyl, butylcyclopentyl, ethylcyclohexyl, butylcyclohexyl, adamantyl, ethyladamantyl, and butyladamantyl, and modified forms of the foregoing in which any carbon-carbon bond is separated by a hetero atomic group such as —O—, —S—, —SO—, —SO₂—, —NH—, —C(═O)—, —C(═O)O— or —C(═O)NH— or in which any hydrogen atom is substituted by a functional group such as —OH, —NH₂, —CHO, or —CO₂H. R⁷ is a straight, branched or cyclic, monovalent hydrocarbon group of 7 to 30 carbon atoms which may contain a heteroatom(s), examples of which are given below, but not limited thereto.

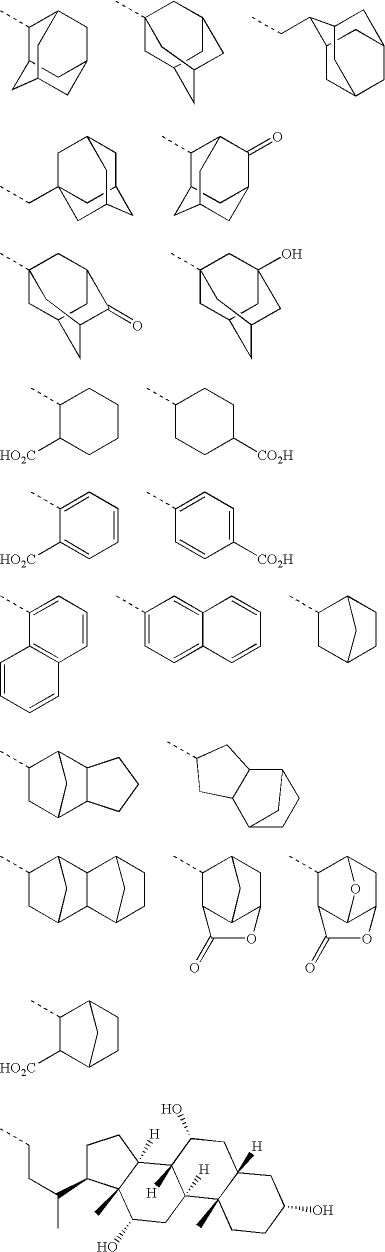

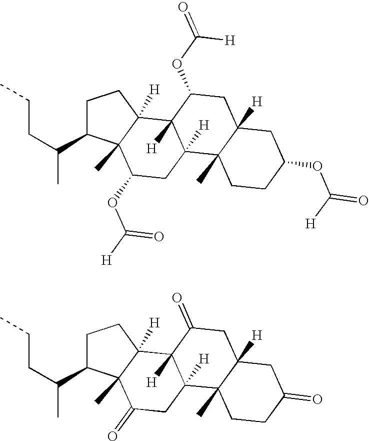

Note that the broken line denotes a valence bond. R⁸ is hydrogen or trifluoromethyl.

Exemplary preferred constructions of the acid generator (B) are given below, but not limited thereto.

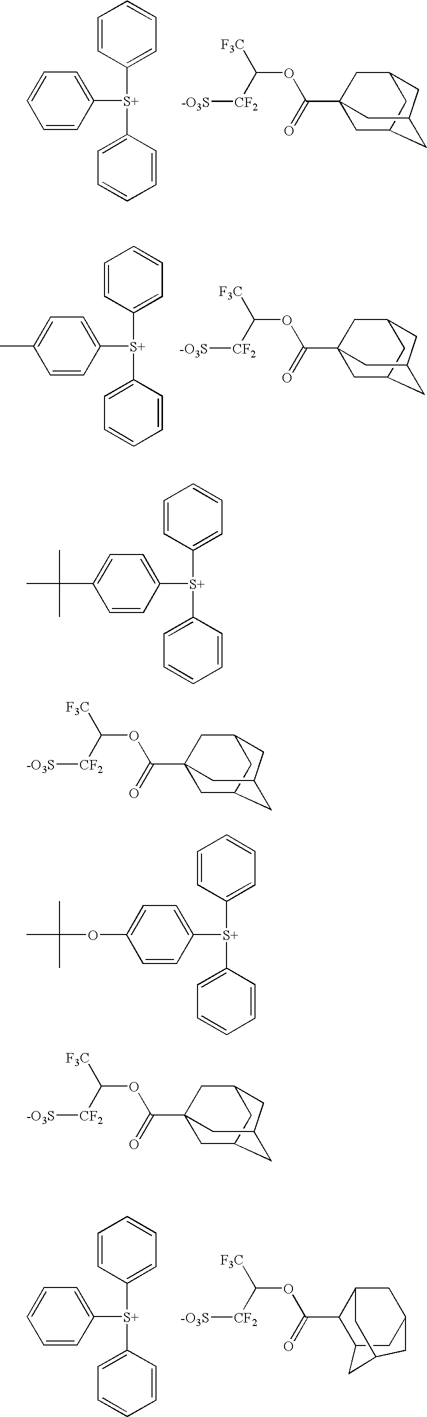

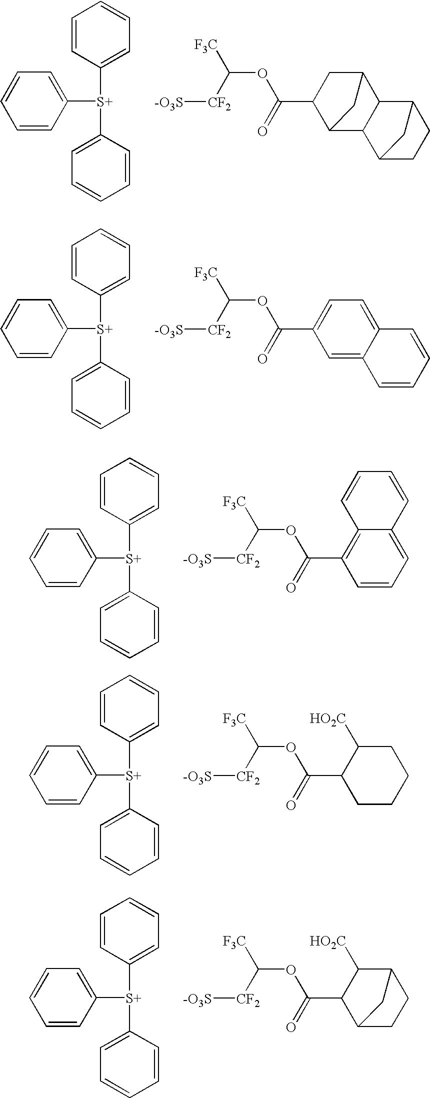

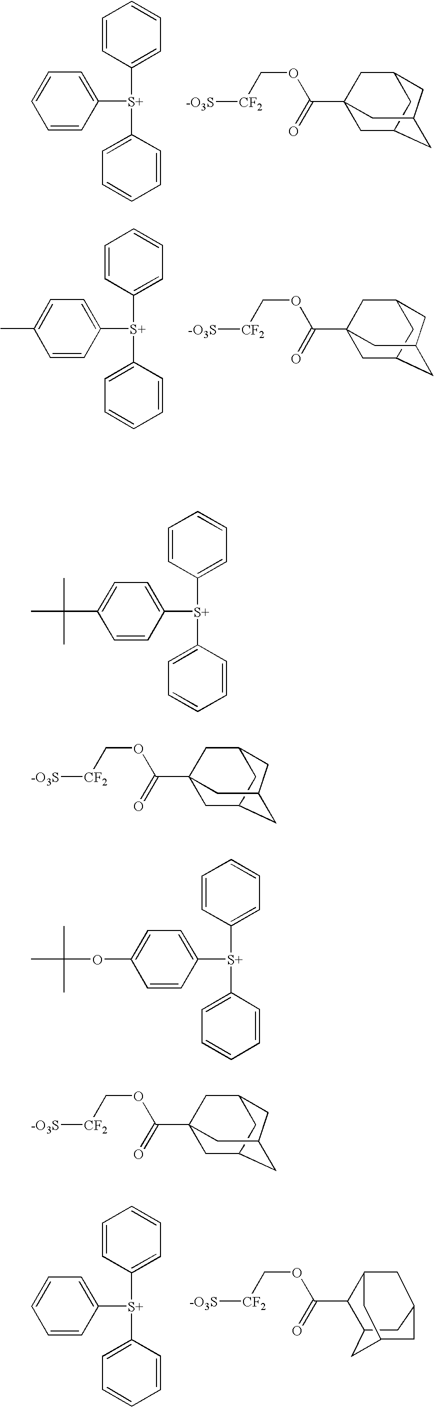

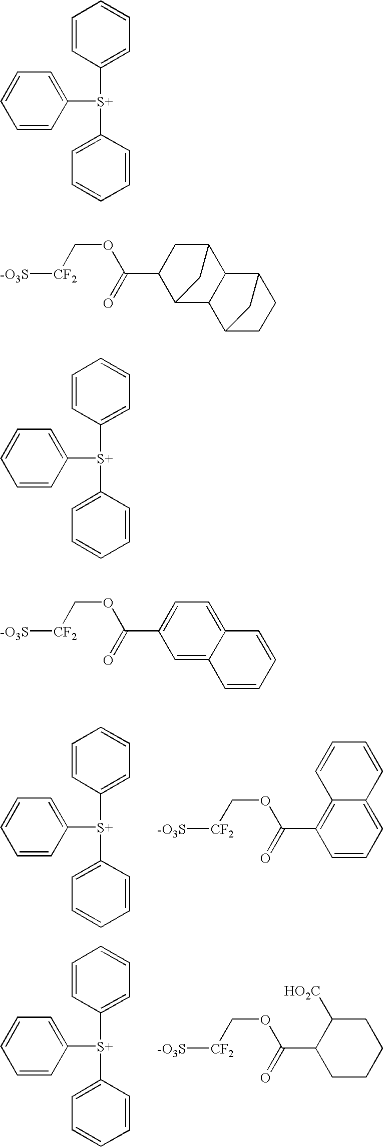

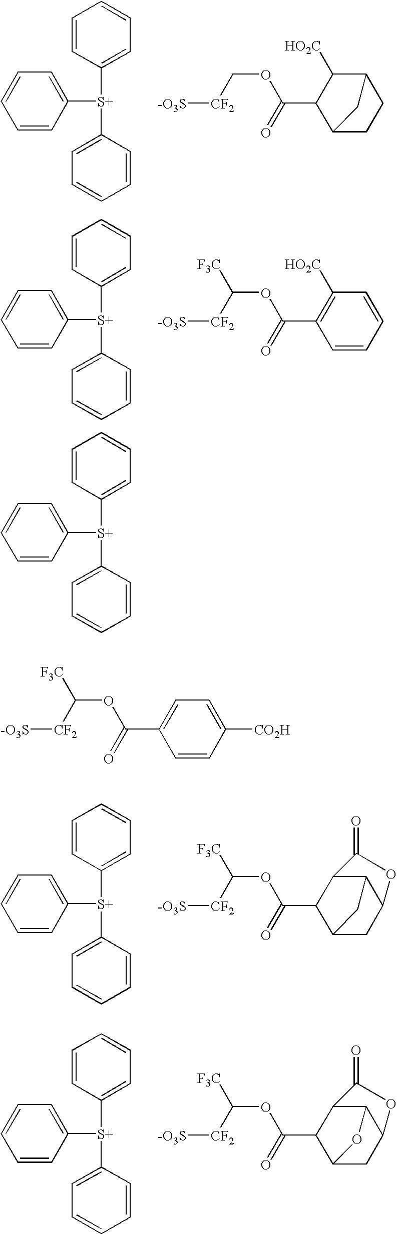

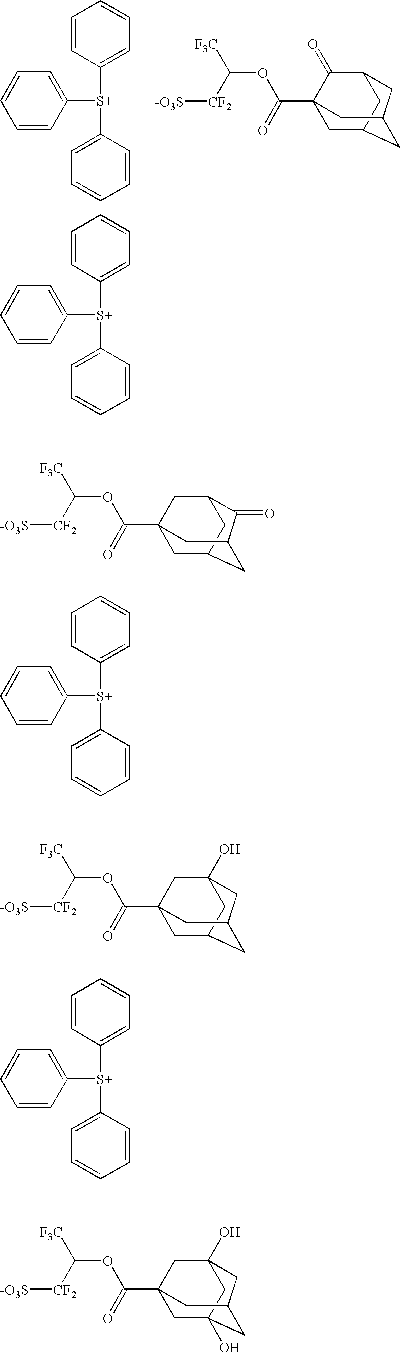

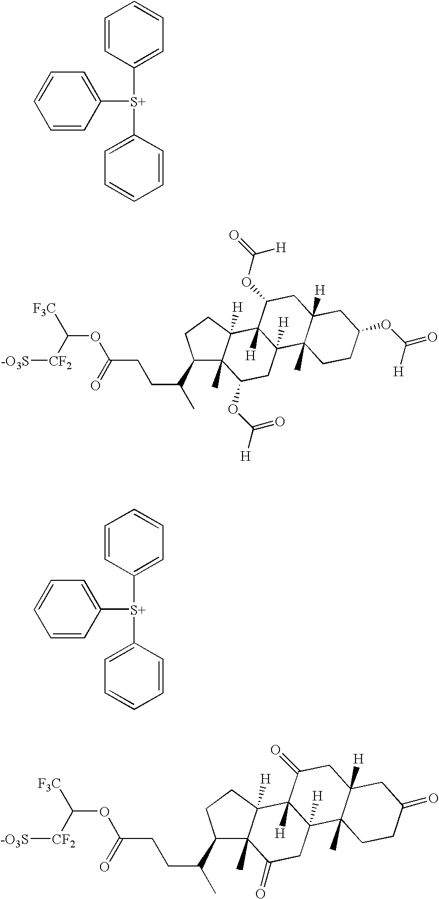

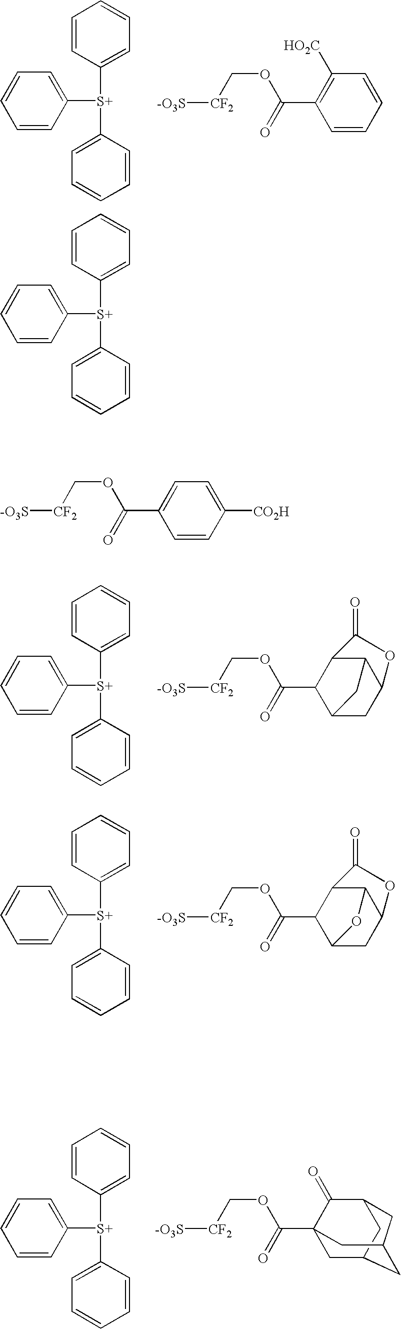

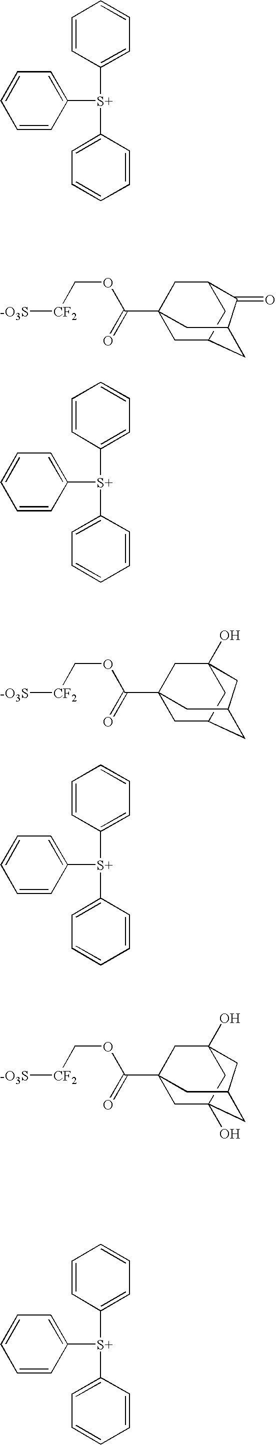

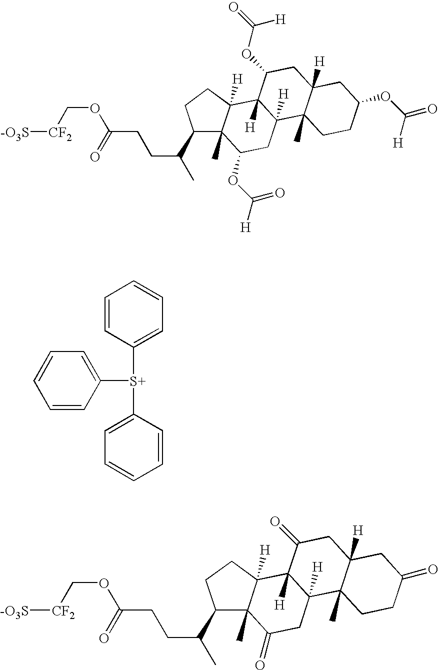

### Other Resin Component

In addition to resin component (A) or polymer comprising non-leaving hydroxyl group-containing recurring units of formula (1), the resist composition of the invention may further comprise another resin component.

The resin component other than resin component (A) that can be added to the resist composition includes, but is not limited to, polymers comprising units of the following formula (R1) and/or (R2) and having a weight average molecular weight of 1,000 to 100,000, especially 3,000 to 30,000, as measured by GPC versus polystyrene standards.

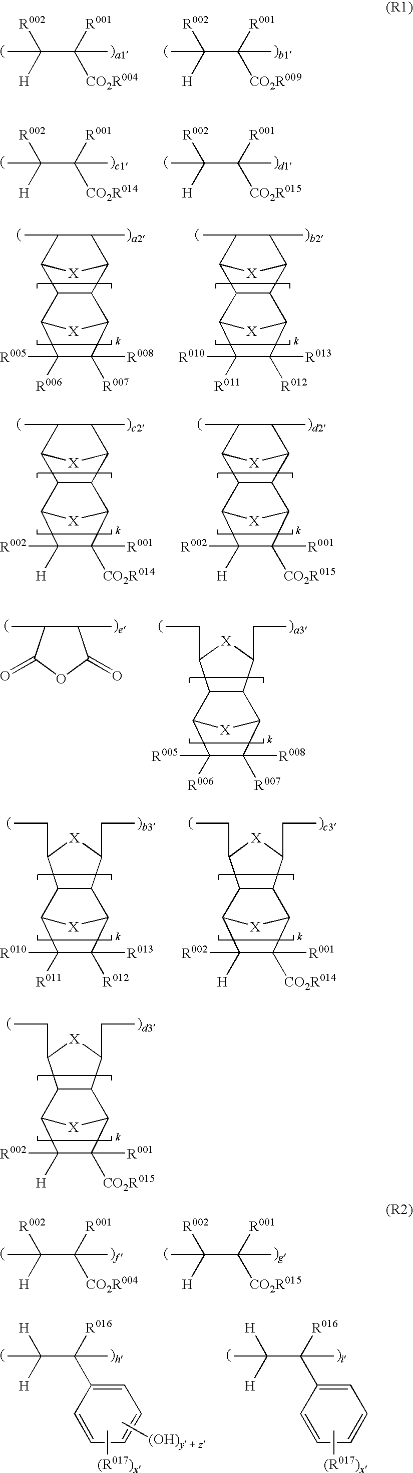

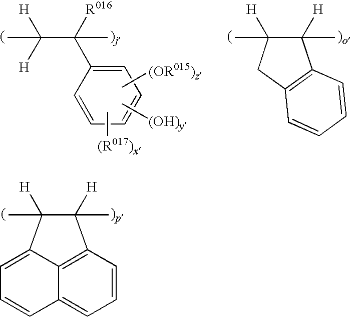

Herein, R⁰⁰¹ is hydrogen, methyl or CH₂CO₂R⁰⁰³. R⁰⁰² is hydrogen, methyl or CO₂R⁰⁰³. R⁰⁰³ is a straight, branched or cyclic C₁-C₁₅ alkyl group, examples of which include methyl, ethyl, propyl, isopropyl, n-butyl, sec-butyl, tert-butyl, tert-amyl, n-pentyl, n-hexyl, cyclopentyl, cyclohexyl, ethylcyclopentyl, butylcyclopentyl, ethylcyclohexyl, butylcyclohexyl, adamantyl, ethyladamantyl, and butyladamantyl.

R⁰⁰⁴ is hydrogen or a monovalent hydrocarbon group of 1 to 15 carbon atoms having at least one group selected from among fluorinated substituent groups, carboxyl groups and hydroxyl groups. Examples include hydrogen, carboxyethyl, carboxybutyl, carboxycyclopentyl, carboxycyclohexyl, carboxynorbornyl, carboxyadamantyl, hydroxyethyl, hydroxybutyl, hydroxycyclopentyl, hydroxycyclohexyl, hydroxynorbornyl, hydroxyadamantyl, [2,2,2-trifluoro-1-hydroxy-1-(trifluoromethyl)ethyl]cyclohexyl, and bis[2,2,2-trifluoro-1-hydroxy-1-(trifluoromethyl)ethyl]-cyclohexyl.

At least one of R⁰⁰⁵ to R⁰⁰⁸ represents a carboxyl group or a monovalent hydrocarbon group of 1 to 15 carbon atoms having at least one group selected from among fluorinated substituent groups, carboxyl groups and hydroxyl groups while the remaining R's independently represent hydrogen or straight, branched or cyclic C₁-C₁₅ alkyl groups. Examples of suitable monovalent C₁-C₁₅ hydrocarbon groups having at least one group selected from among fluorinated substituent groups, carboxyl groups and hydroxyl groups include carboxymethyl, carboxyethyl, carboxybutyl, hydroxymethyl, hydroxyethyl, hydroxybutyl, 2-carboxyethoxycarbonyl, 4-carboxybutoxycarbonyl, 2-hydroxyethoxycarbonyl, 4-hydroxybutoxycarbonyl, carboxycyclopentyloxycarbonyl, carboxycyclohexyloxycarbonyl, carboxynorbornyloxycarbonyl, carboxyadamantyloxycarbonyl, hydroxycyclopentyloxycarbonyl, hydroxycyclohexyloxycarbonyl, hydroxynorbornyloxycarbonyl, hydroxyadamantyloxycarbonyl, [2,2,2-trifluoro-1-hydroxy-1-(trifluoromethyl)ethyl]cyclo-hexyloxycarbonyl, and bis[2,2,2-trifluoro-1-hydroxy-1-(trifluoromethyl)ethyl]cyclo-hexyloxycarbonyl. Suitable straight, branched or cyclic C₁-C₁₅ alkyl groups are as exemplified for R⁰⁰³.

Two of R⁰⁰⁵ to R⁰⁰⁸ (for example, a pair of R⁰⁰⁵ and R⁰⁰⁶, R⁰⁰⁶ and R⁰⁰⁷, or R⁰⁰⁷ and R⁰⁰⁸) may bond together to form a ring with the carbon atom(s) to which they are attached, and in that event, at least one of ring-forming R⁰⁰⁵ to R⁰⁰⁸ is a divalent hydrocarbon group of 1 to 15 carbon atoms having at least one group selected from fluorinated substituent groups, carboxyl groups and hydroxyl groups, while the remaining R's are independently single bonds, hydrogen atoms or straight, branched or cyclic C₁-C₁₅ alkyl groups. Suitable divalent C₁-C₁₅ hydrocarbon groups having at least one group selected from fluorinated substituent groups, carboxyl groups and hydroxyl groups include those exemplified above as the monovalent hydrocarbon groups having at least one group selected from fluorinated substituent groups, carboxyl groups and hydroxyl groups, with one hydrogen atom eliminated therefrom. Suitable straight, branched or cyclic C₁-C₁₅ alkyl groups are as exemplified for R⁰⁰³.

R⁰⁰⁹ is a monovalent hydrocarbon group of 3 to 15 carbon atoms containing a —CO₂— partial structure. Examples include 2-oxooxolan-3-yl, 4,4-dimethyl-2-oxooxolan-3-yl, 4-methyl-2-oxooxan-4-yl, 2-oxo-1,3-dioxolan-4-ylmethyl, and 5-methyl-2-oxooxolan-5-yl.

At least one of R⁰¹⁰ to R⁰¹³ is a monovalent hydrocarbon group of 2 to 15 carbon atoms containing a —CO₂— partial structure, while the remaining R's are independently hydrogen atoms or straight, branched or cyclic C₁-C₁₅ alkyl groups. Illustrative examples of suitable monovalent C₂-C₁₅ hydrocarbon groups containing a —CO₂— partial structure include 2-oxooxolan-3-yloxycarbonyl, 4,4-dimethyl-2-oxooxolan-3-yloxycarbonyl, 4-methyl-2-oxooxan-4-yloxycarbonyl, 2-oxo-1,3-dioxolan-4-ylmethyloxycarbonyl, and 5-methyl-2-oxooxolan-5-yloxycarbonyl. Suitable straight, branched or cyclic C₁-C₁₅ alkyl groups are as exemplified for R⁰⁰³.

Two of R⁰¹⁰ to R⁰¹³ (for example, a pair of R⁰¹⁰ and R⁰¹¹, R⁰¹¹ and R⁰¹², or R⁰¹² and R⁰¹³) may bond together to form a ring with the carbon atom(s) to which they are attached, and in that event, at least one of ring-forming R⁰¹⁰ to R⁰¹³ is a divalent hydrocarbon group of 1 to 15 carbon atoms containing a —CO₂— partial structure, while the remaining R's are independently single bonds, hydrogen atoms or straight, branched or cyclic C₁-C₁₅ alkyl groups. Illustrative examples of suitable divalent C₁-C₁₅ hydrocarbon groups containing a —CO₂— partial structure include 1-oxo-2-oxapropane-1,3-diyl, 1,3-dioxo-2-oxapropane-1,3-diyl, 1-oxo-2-oxabutane-1,4-diyl, and 1,3-dioxo-2-oxabutane-1,4-diyl, as well as those exemplified as the monovalent hydrocarbon groups containing a —CO₂— partial structure, with one hydrogen atom eliminated therefrom. Suitable straight, branched or cyclic C₁-C₁₅ alkyl groups are as exemplified for R⁰⁰³.

R⁰¹⁴ is a polycyclic hydrocarbon group having 7 to 15 carbon atoms or an alkyl group containing such a polycyclic hydrocarbon group. Examples include norbornyl, bicyclo[3.3.1]nonyl, tricyclo[5.2.1.0^{2,6}]decyl, adamantyl, norbornylmethyl, and adamantylmethyl as well as alkyl- or cycloalkyl-substituted forms of the foregoing. R⁰¹⁵ is an acid labile group as illustrated for R².

R⁰¹⁶ is hydrogen or methyl. R⁰¹⁷ is a straight, branched or cyclic C₁-C₈ alkyl group, examples of which include methyl, ethyl, propyl, isopropyl, n-butyl, sec-butyl, tert-butyl, tert-amyl, n-pentyl, n-hexyl, cyclopentyl and cyclohexyl.

X is CH₂ or an oxygen atom. Letter k is 0 or 1.

In formulae (R1) and (R2), letters a1′, a2′, a3′, b1′, b2′, b3′, c1′, c2′, c3′, d1′, d2′, d3′, and e′ are numbers from 0 to less than 1, satisfying a1′+a2′+a3′+b1′+b2′+b3′+c1′+c2′+c3′+d1′+d2′+d3′+e′=1; f′, g′, h′, i′, j′, o′, and p′ are numbers from 0 to less than 1, satisfying f′+g′+h′+i′+j′+o′+p′=1; x′, y′ and z′ are each an integer of 0 to 3, satisfying 1≦x′+y′+z′≦5 and 1≦y′+z′≦3.

With respect to the recurring units of formula (R1) and (R2), units of more than one type may be incorporated at the same time. Incorporation of units of more than one type enables to adjust the performance of a resist material in which the resulting polymer is formulated.

Understandably, the sum of respective units=1 means that in a polymer comprising recurring units, the total of the indicated recurring units is 100 mol % relative to the total of entire recurring units.

Examples of the recurring units incorporated at compositional ratio a1′ in formula (R1) and the recurring units incorporated at compositional ratio f′ in formula (R2) are given below, but not limited thereto.

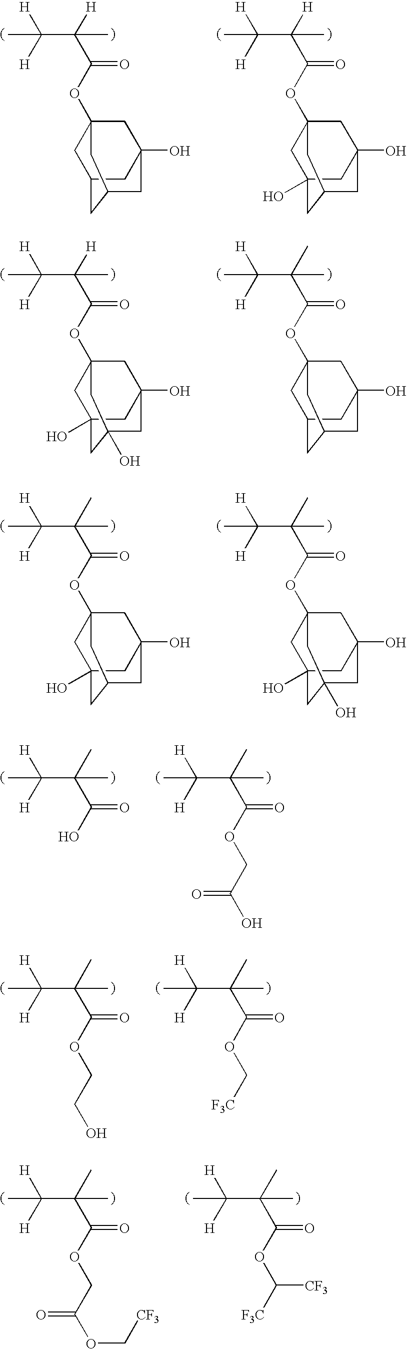

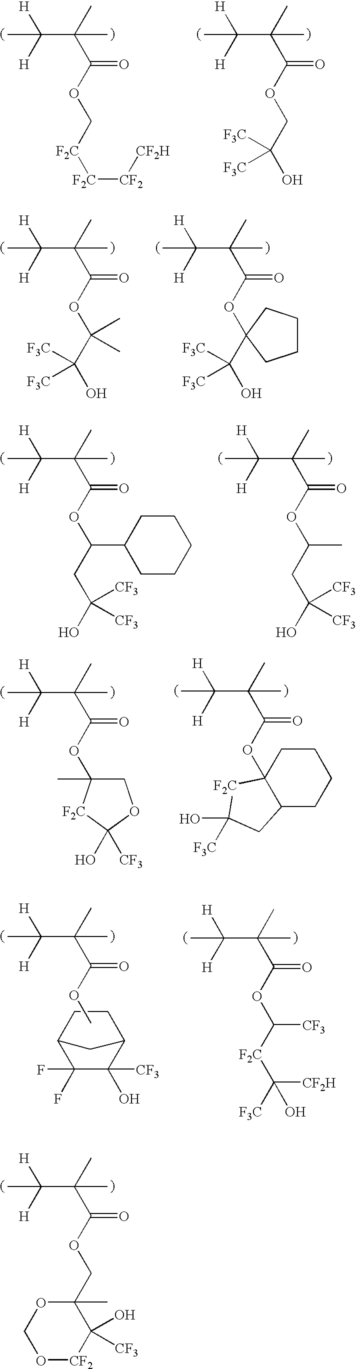

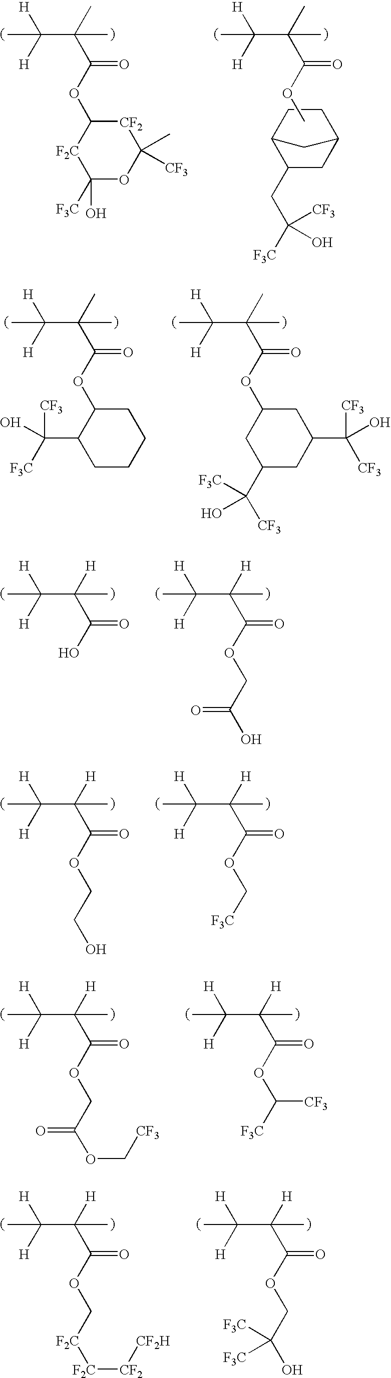

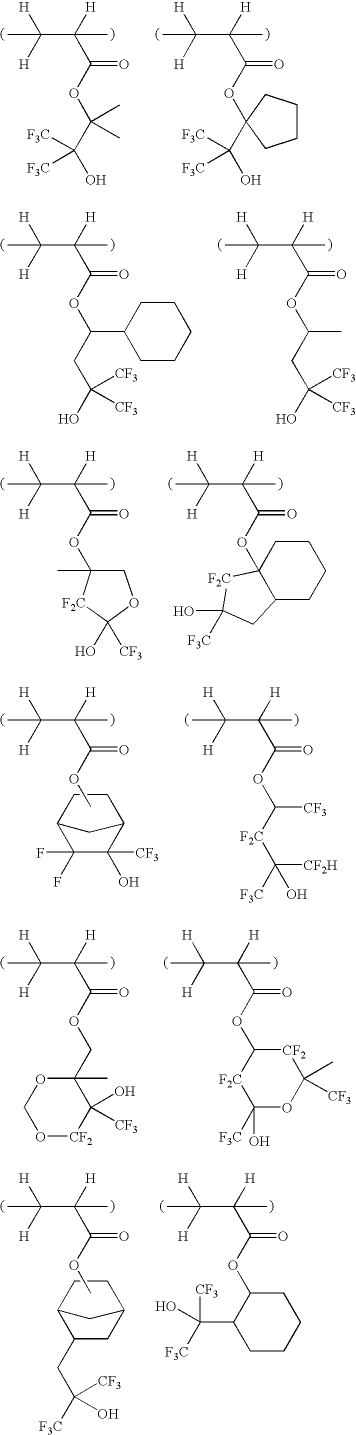

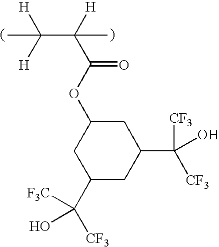

Examples of the recurring units incorporated at compositional ratio b1′ in formula (R1) are given below, but not limited thereto.

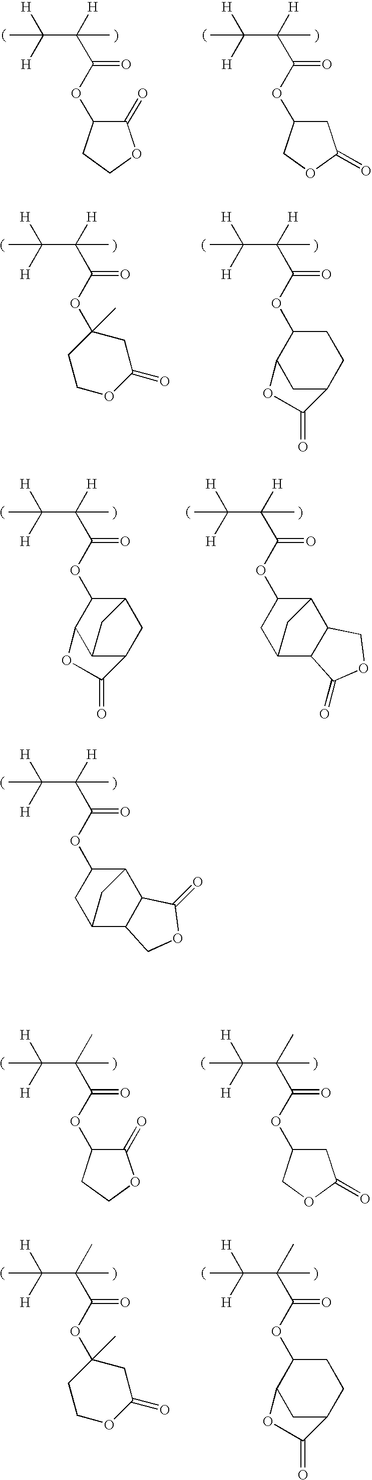

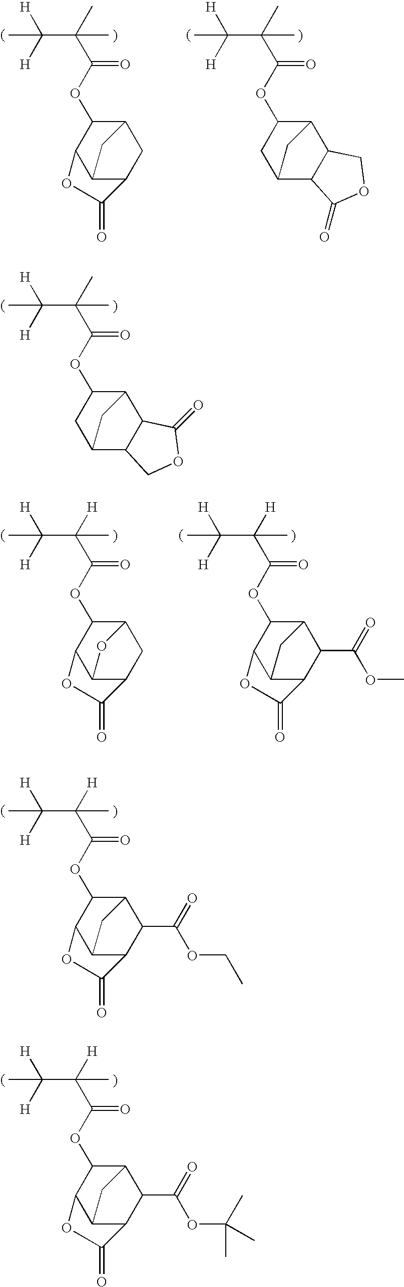

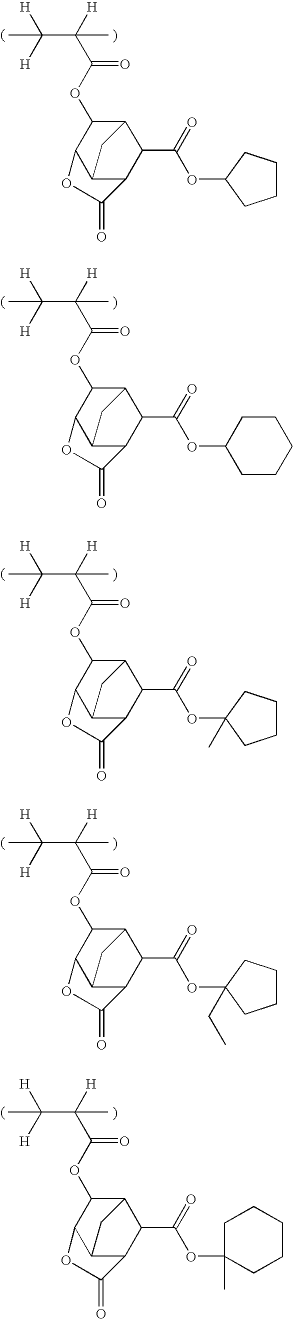

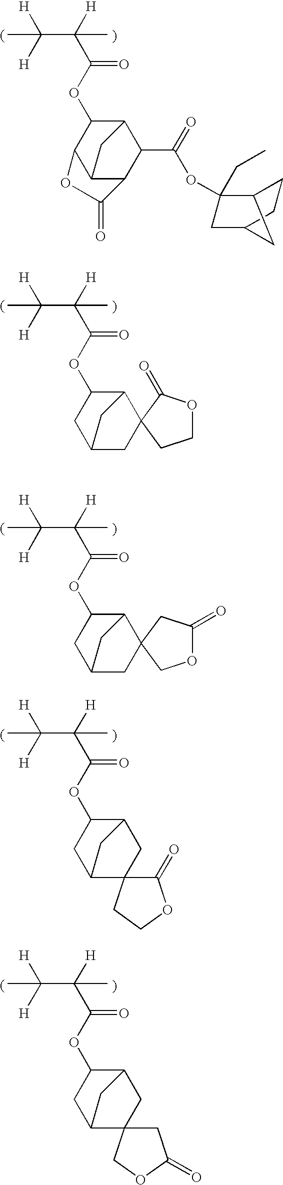

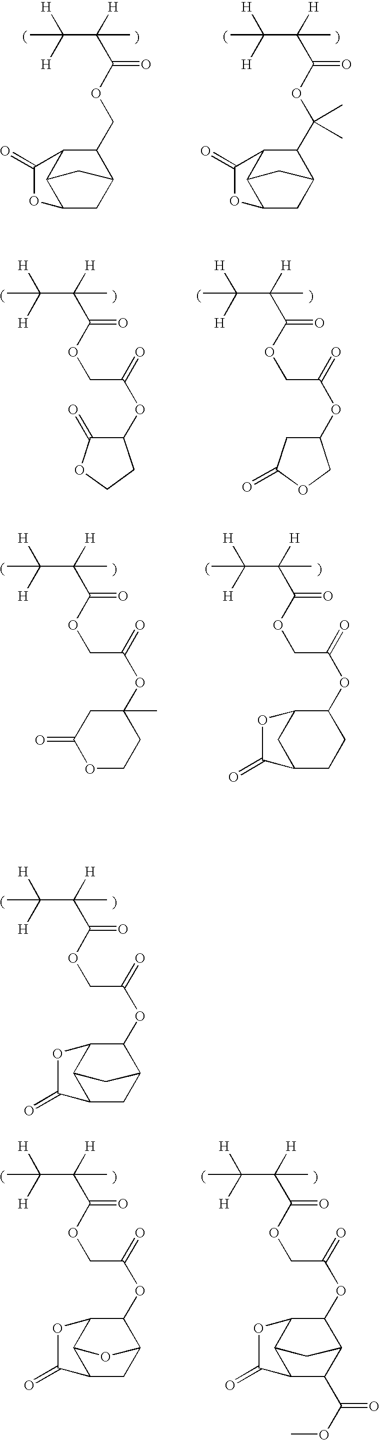

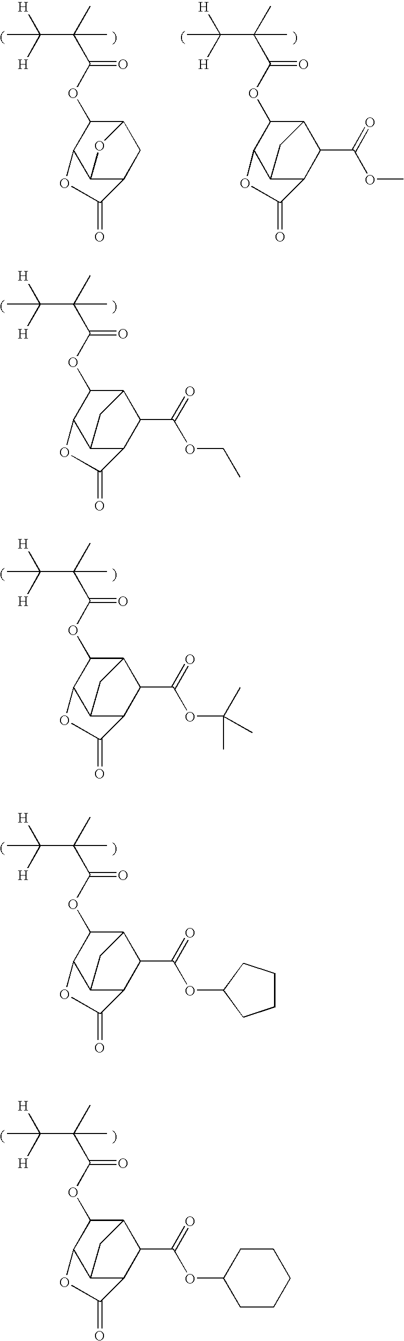

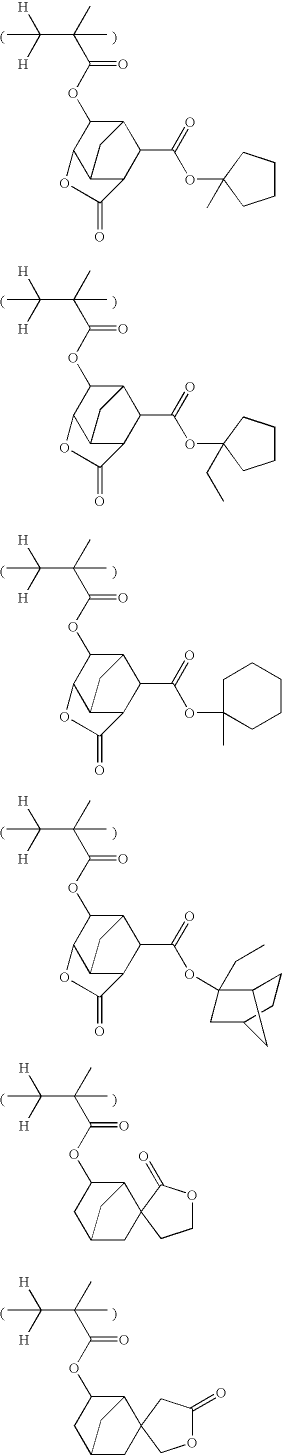

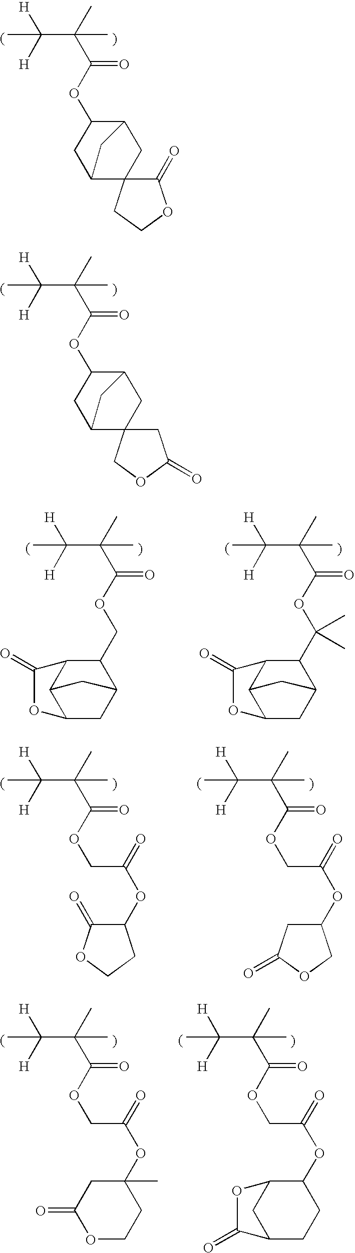

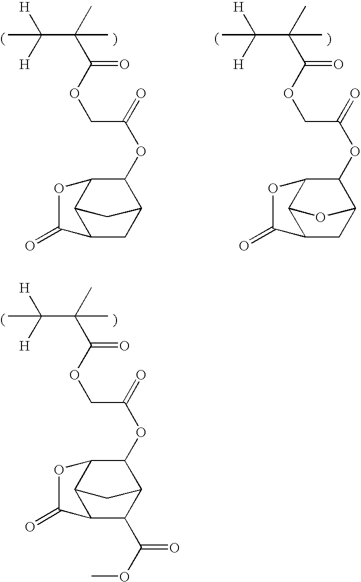

Examples of the recurring units incorporated at compositional ratio d1′ in formula (R1) and the recurring units incorporated at compositional ratio g′ in formula (R2) are given below, but not limited thereto.

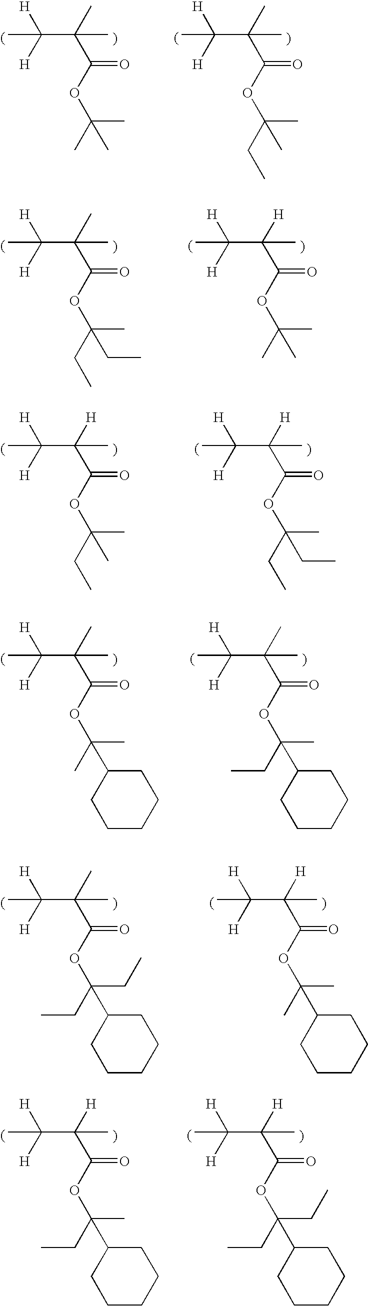

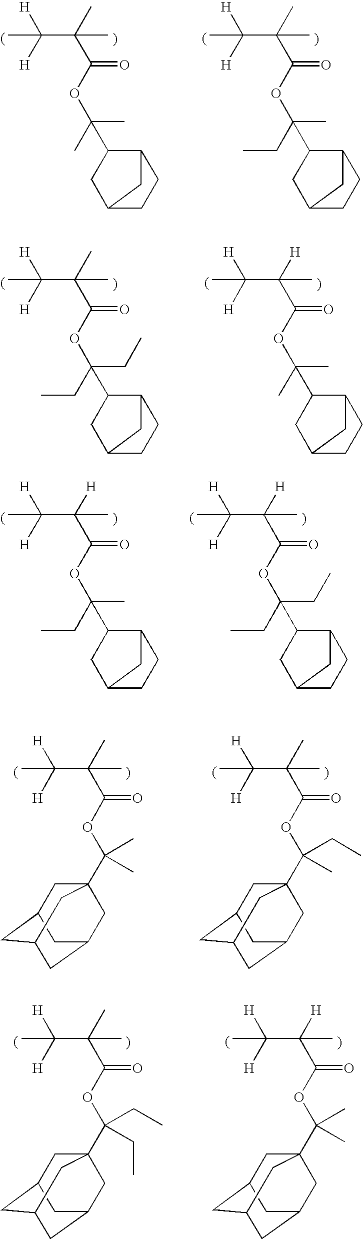

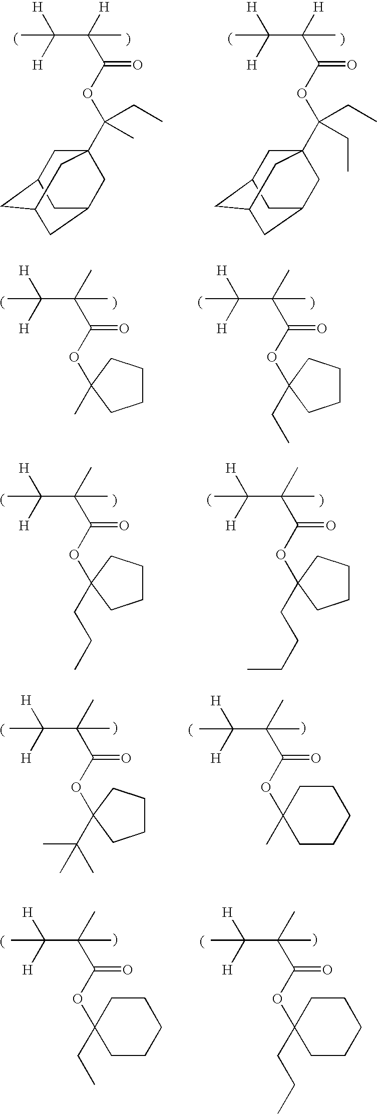

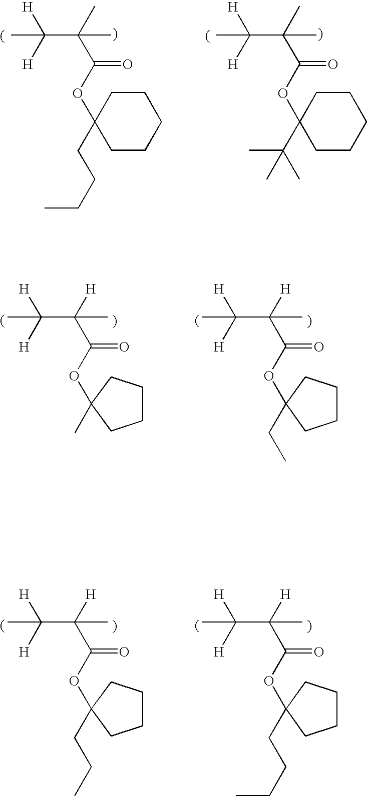

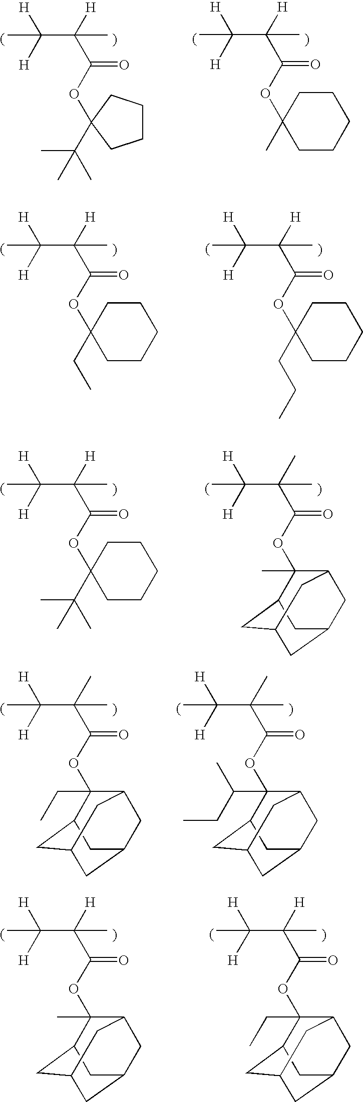

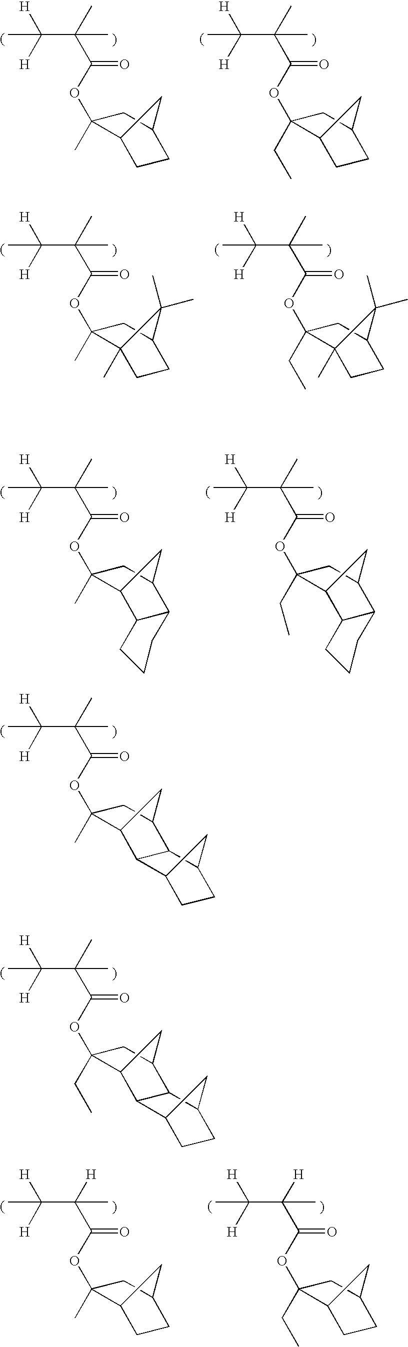

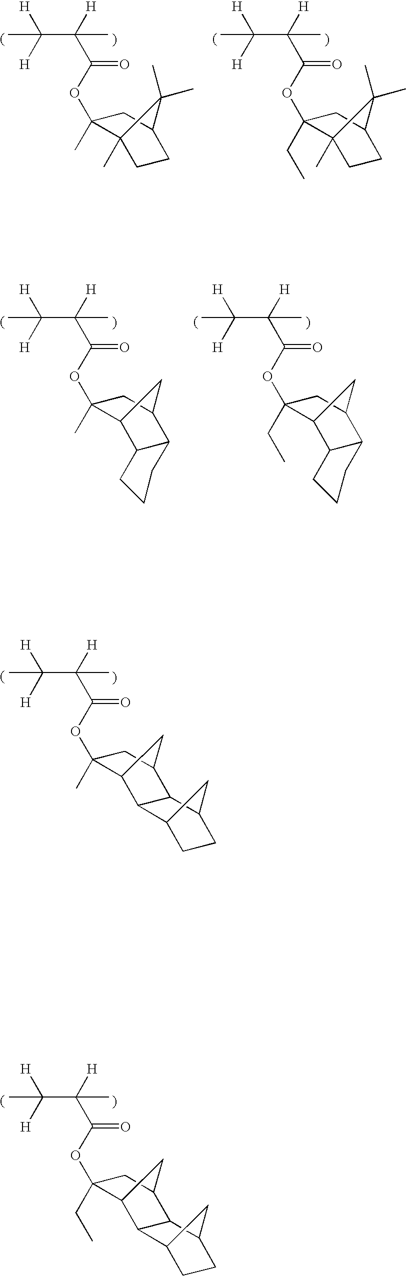

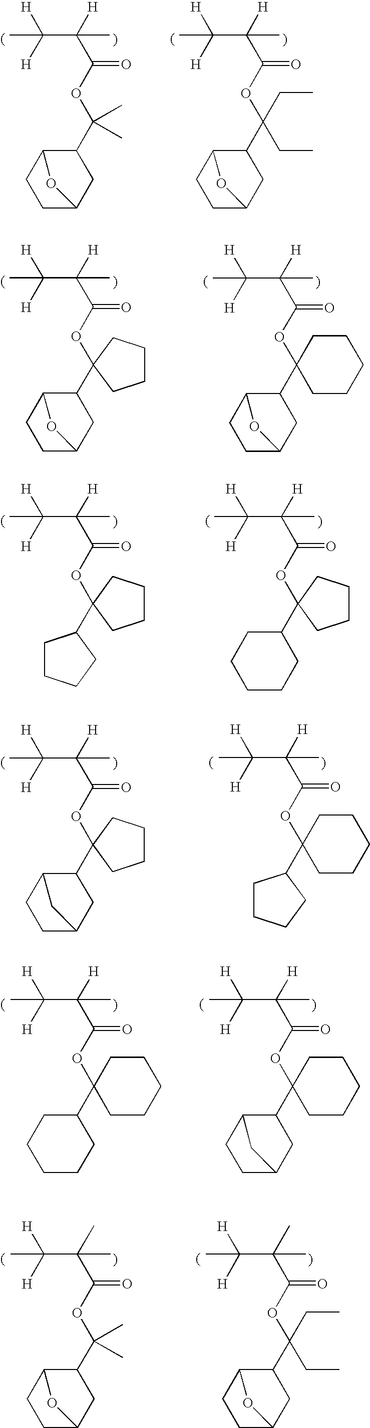

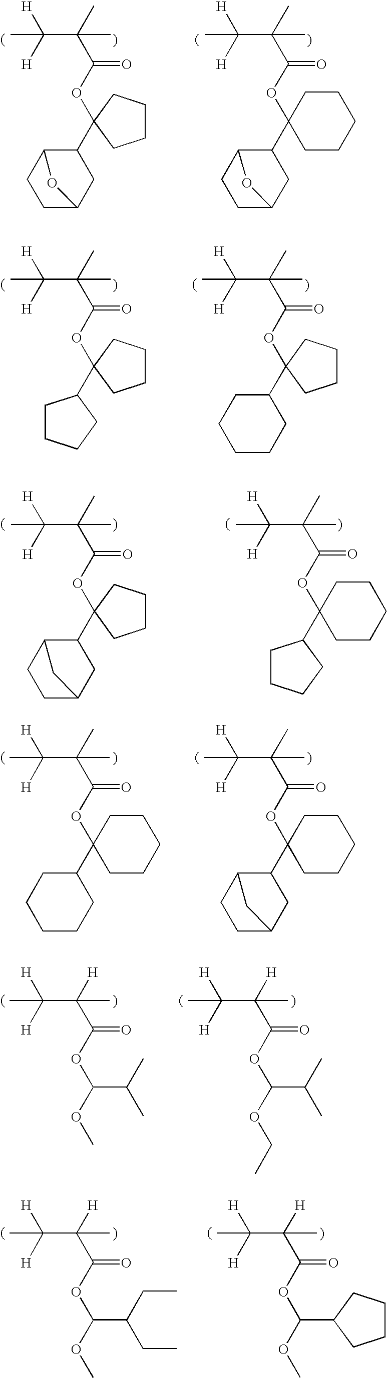

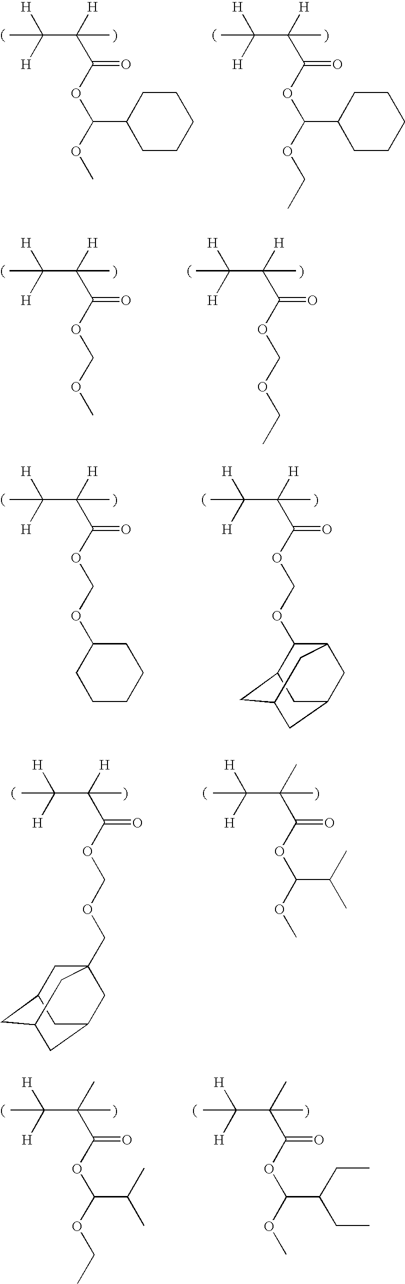

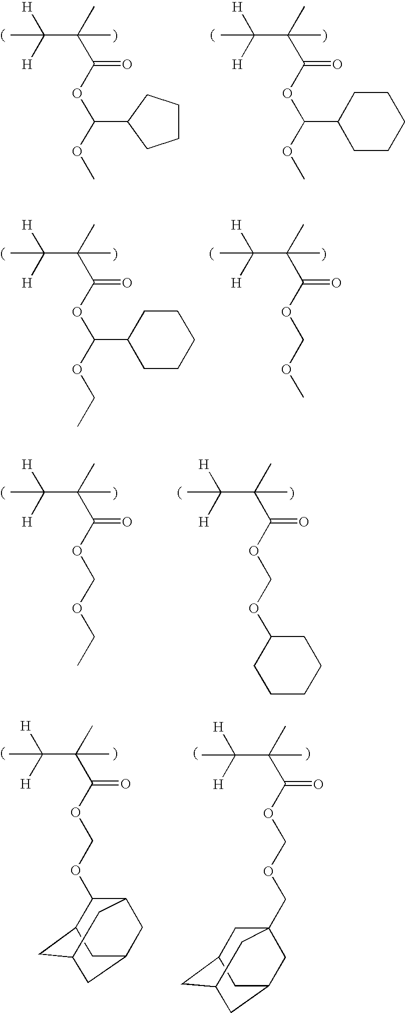

Exemplary polymers comprising recurring units incorporated at compositional ratios a1′, b1′, c1′, and d1′ in formula (R1) are shown below, but not limited thereto.

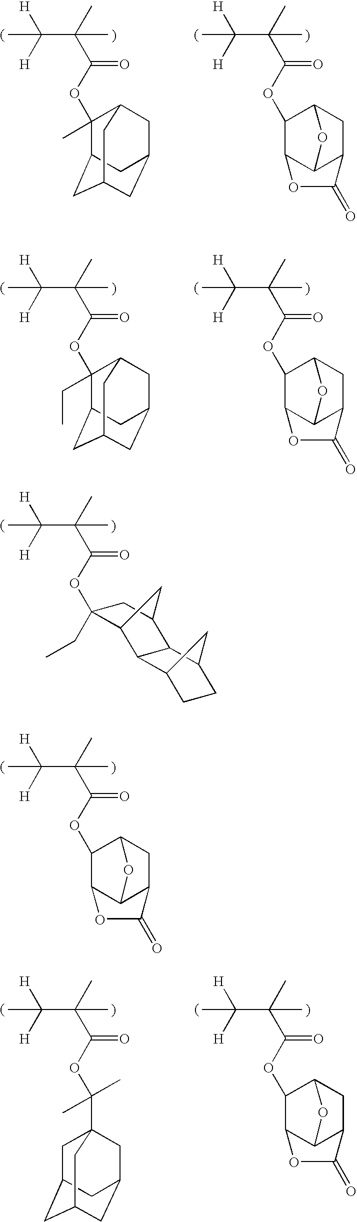

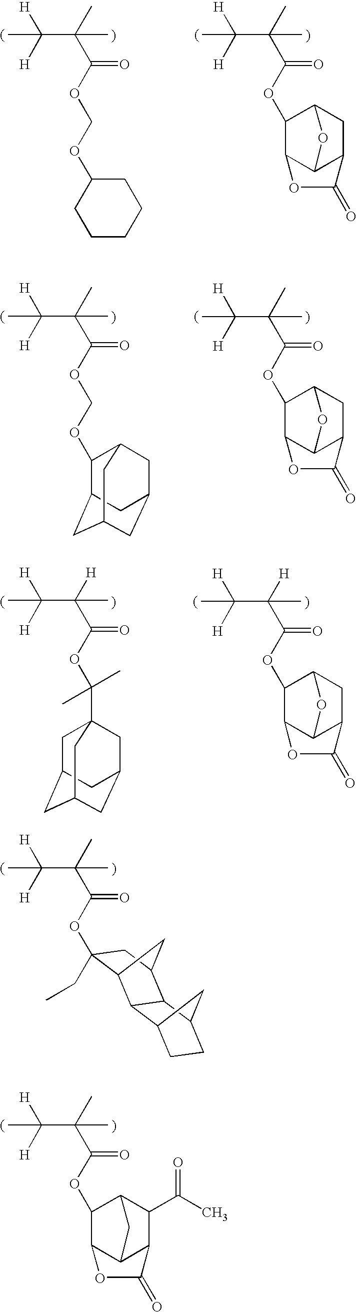

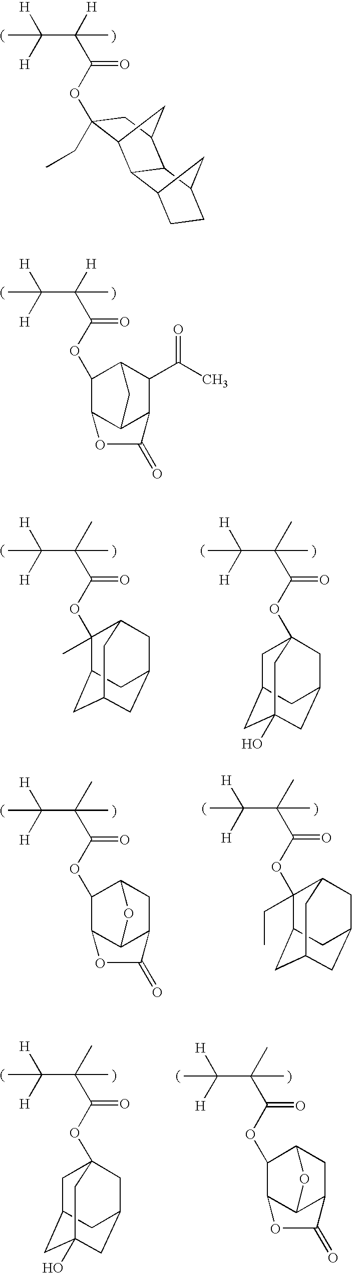

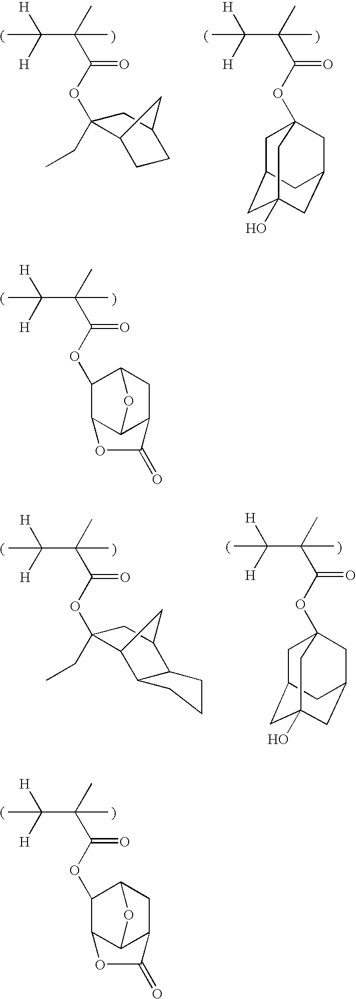

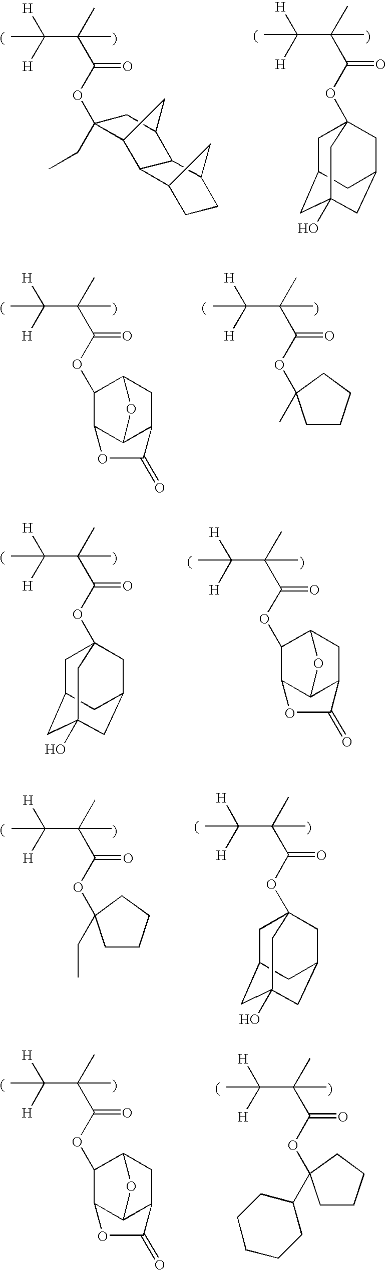

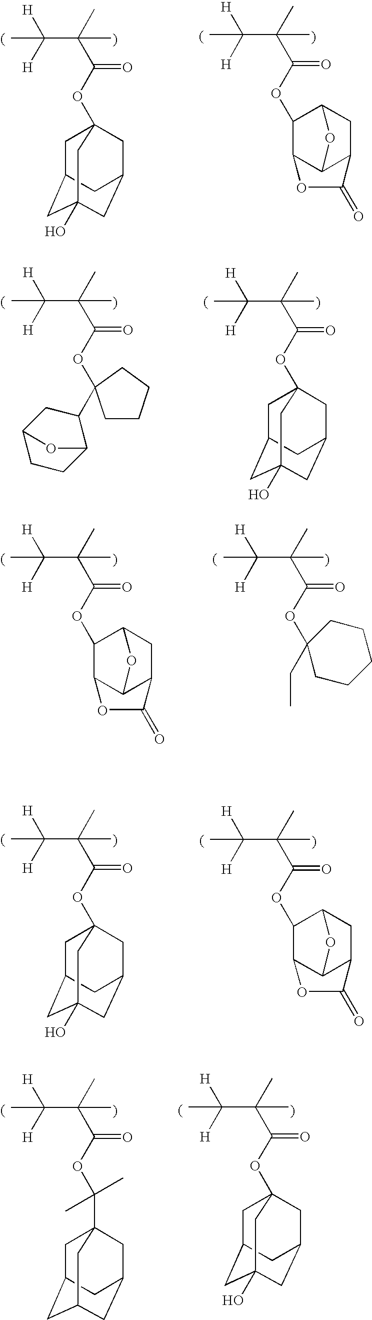

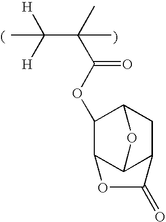

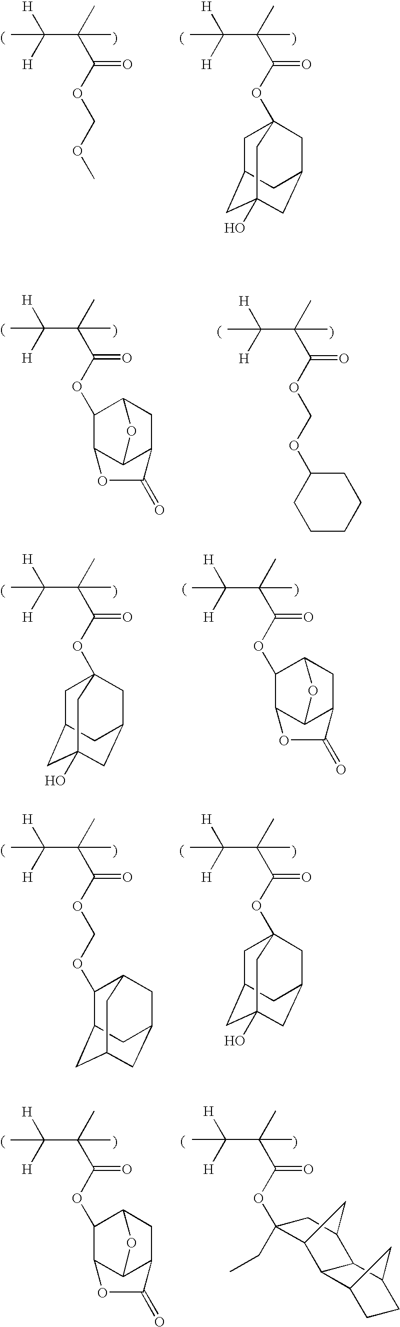

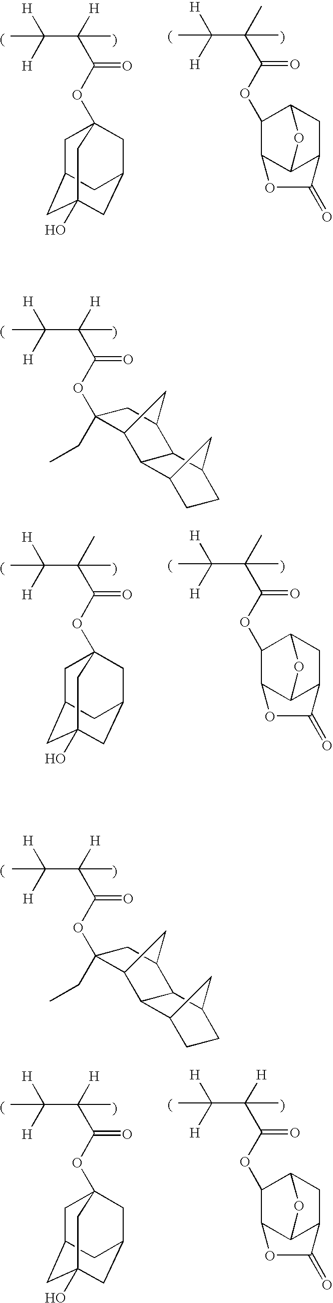

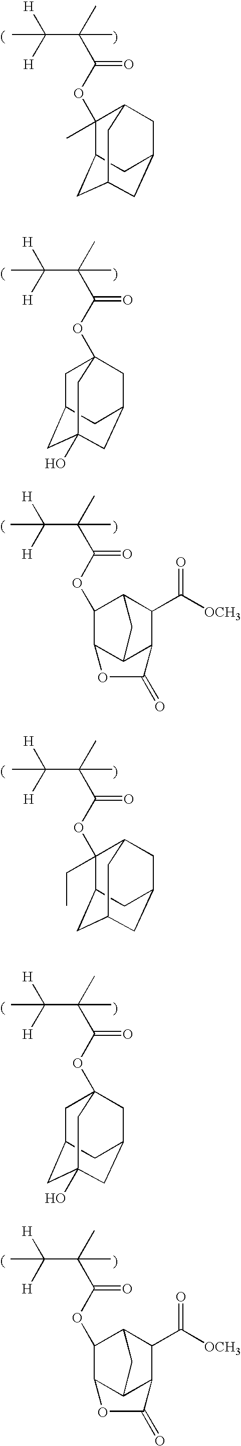

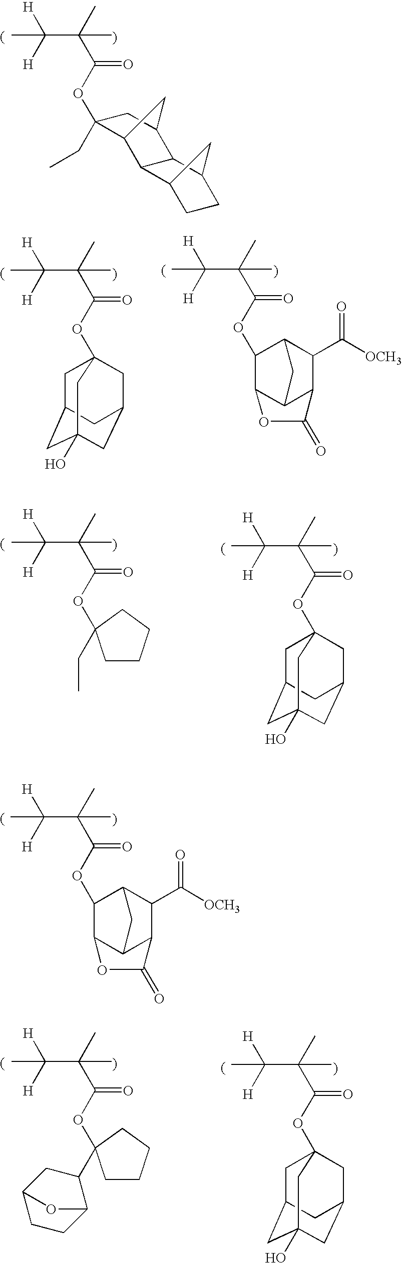

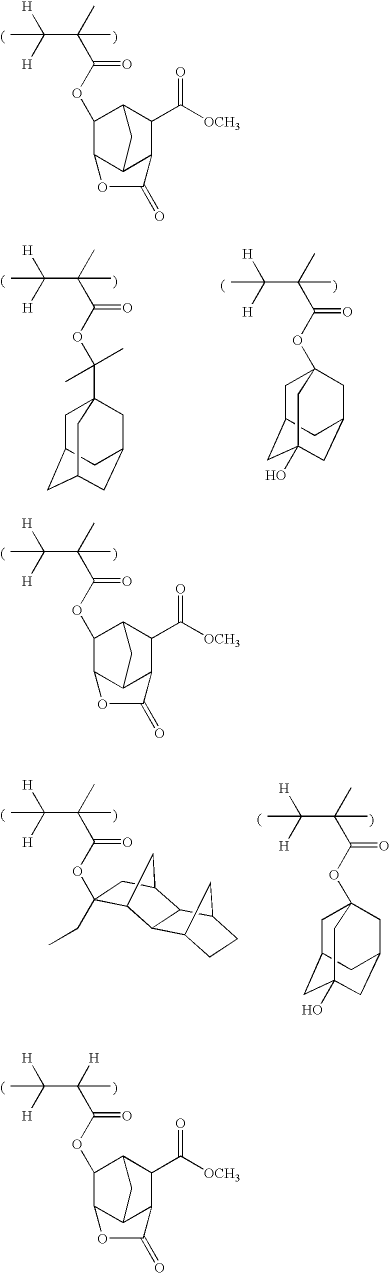

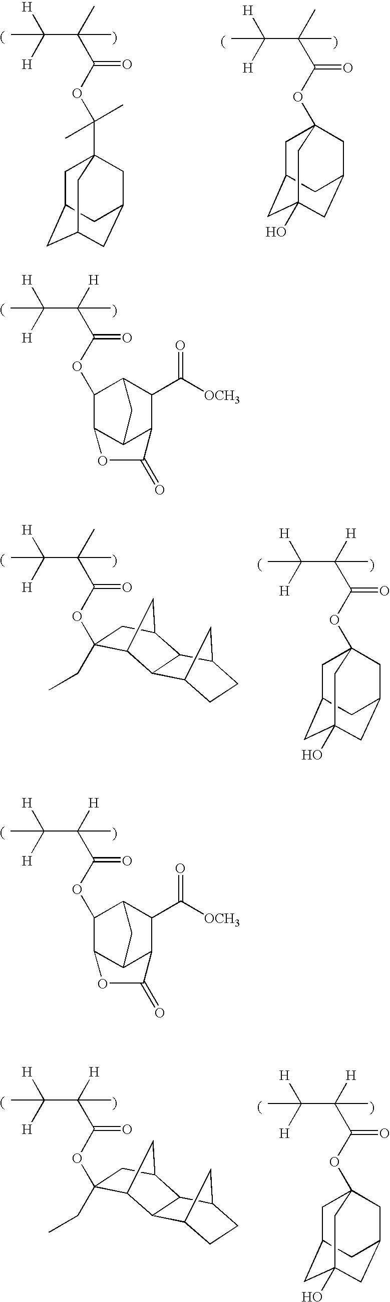

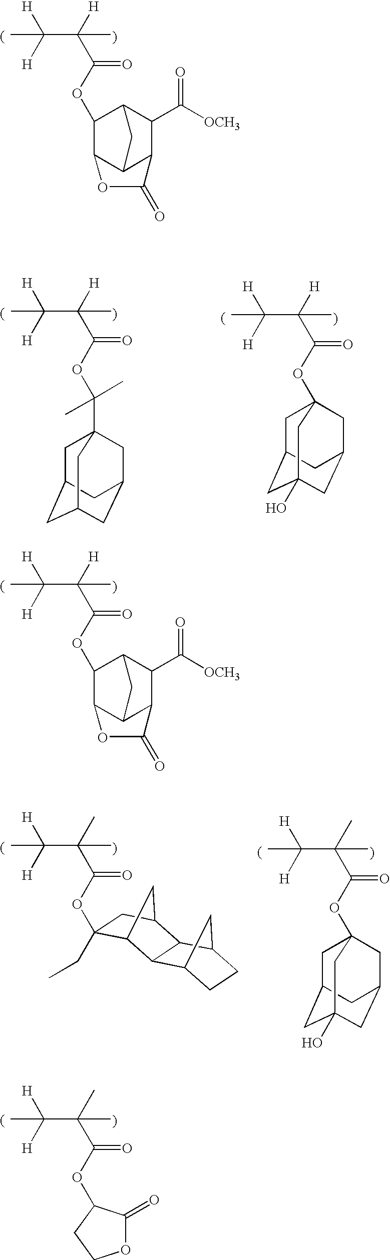

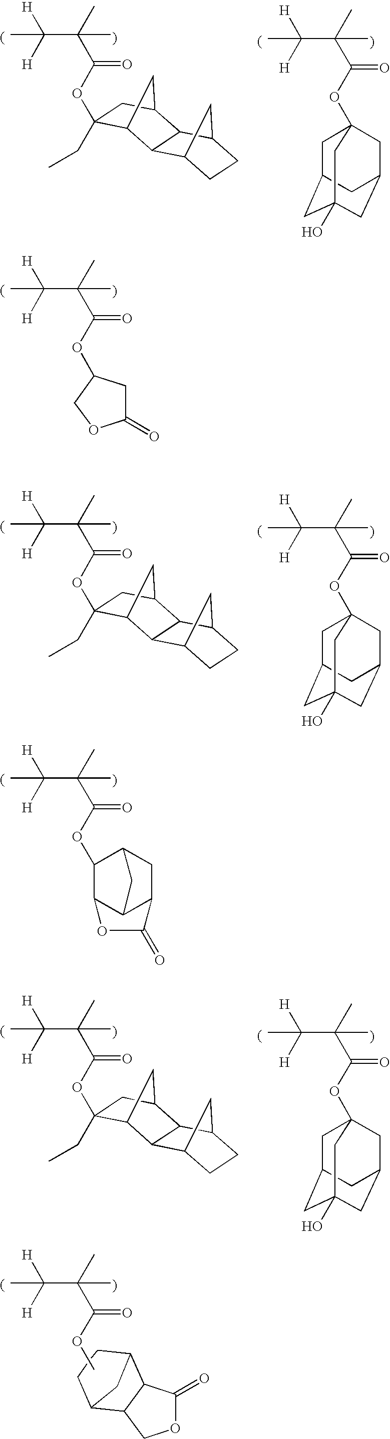

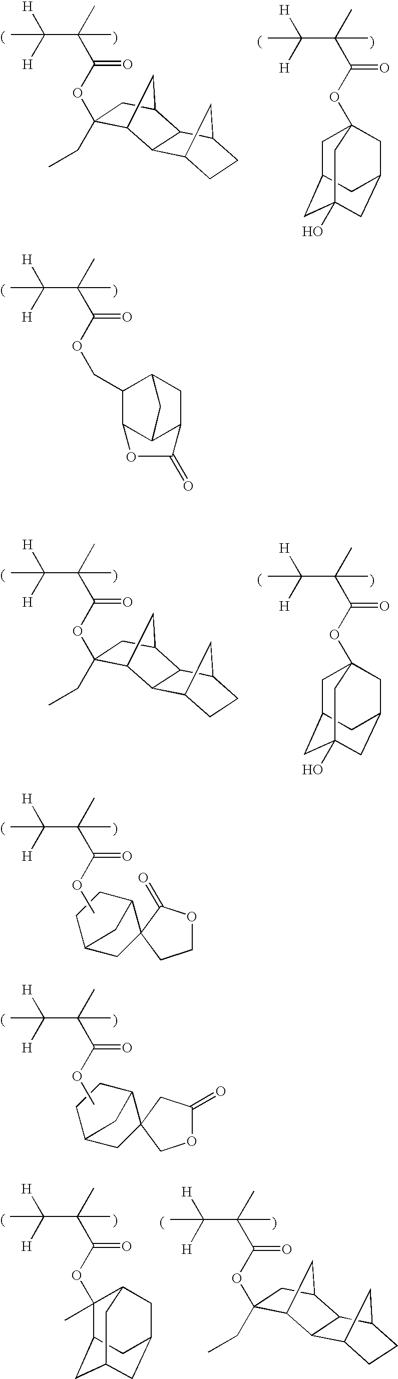

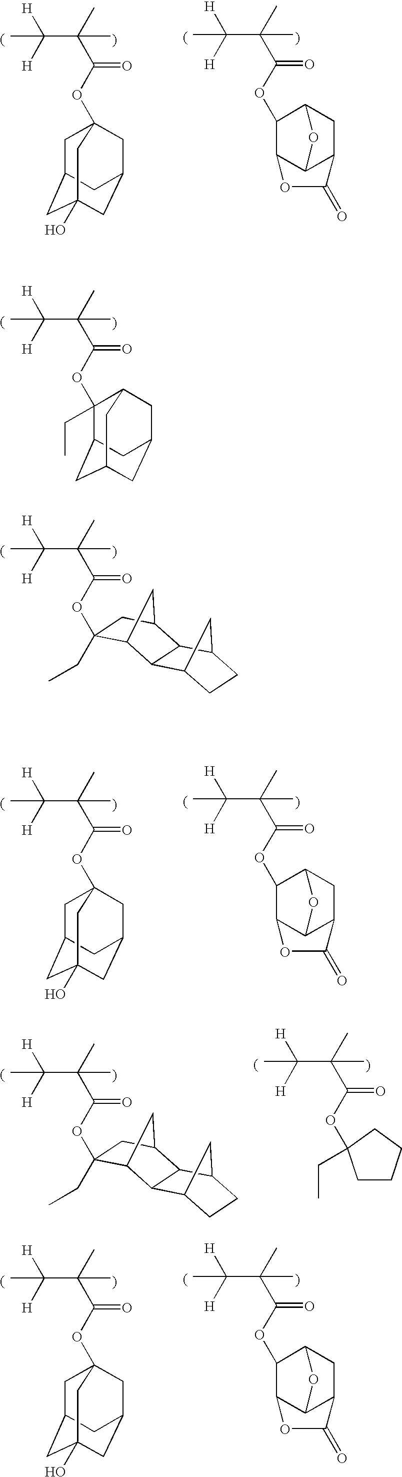

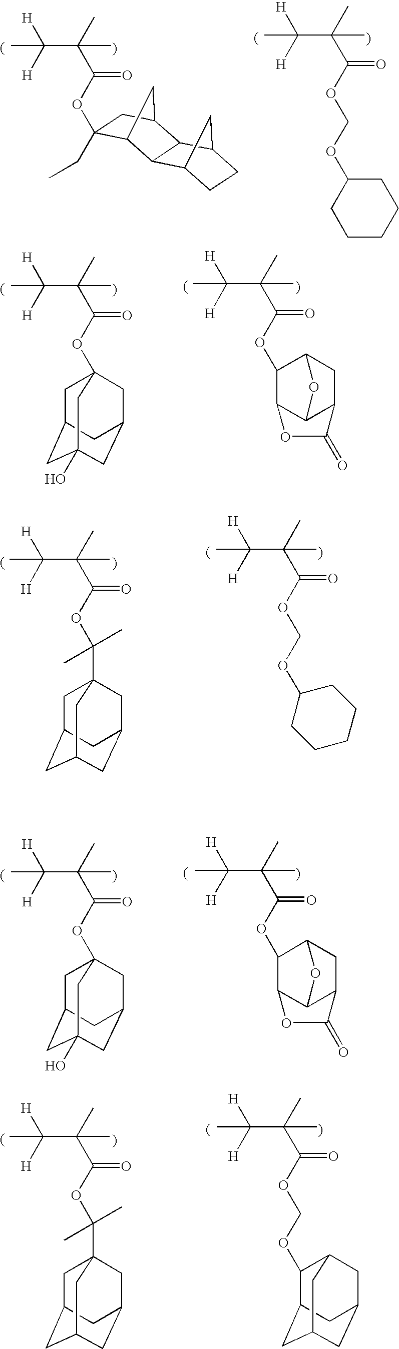

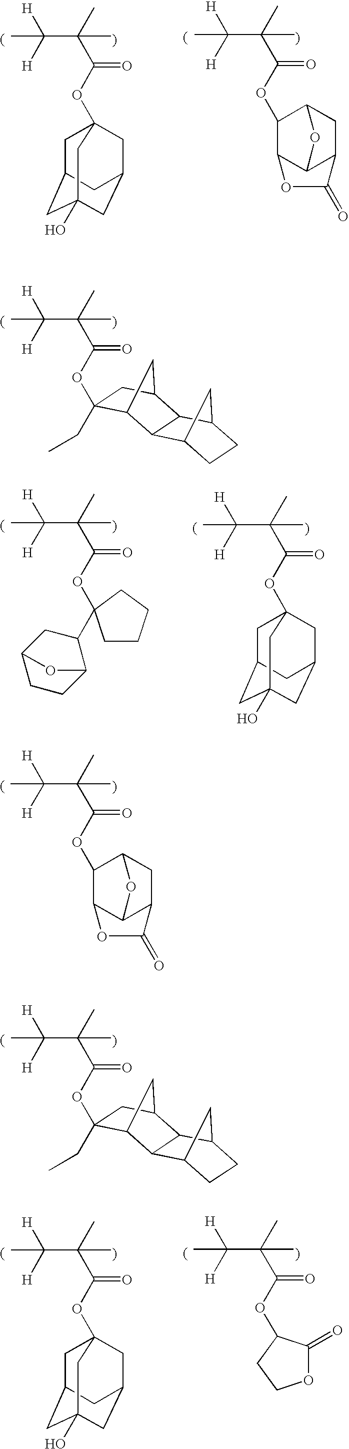

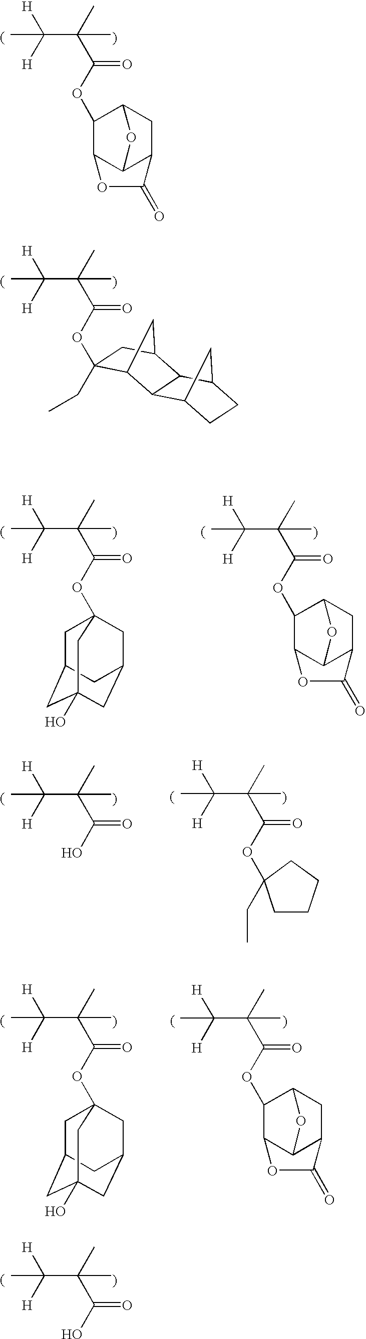

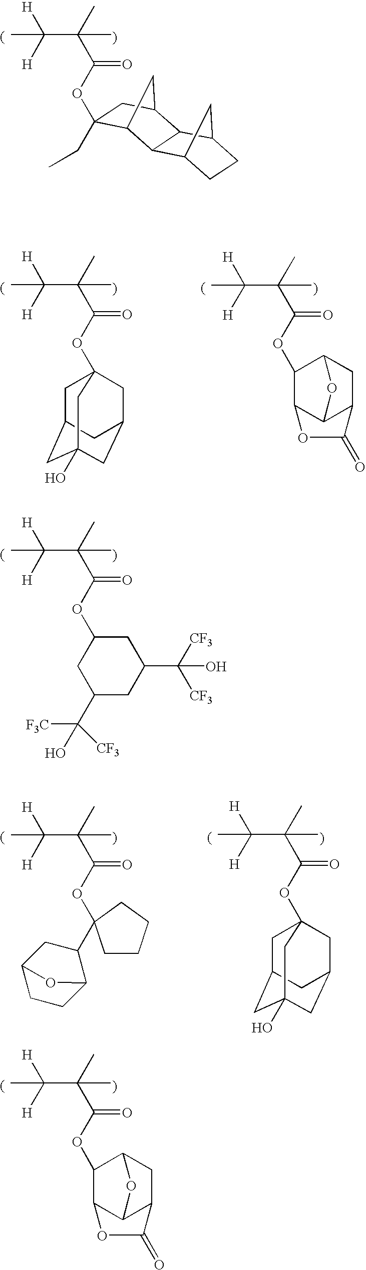

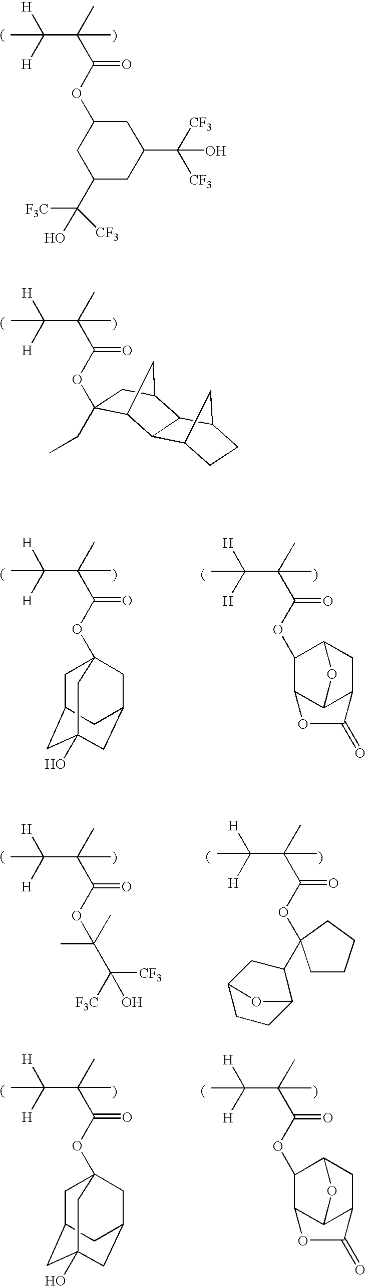

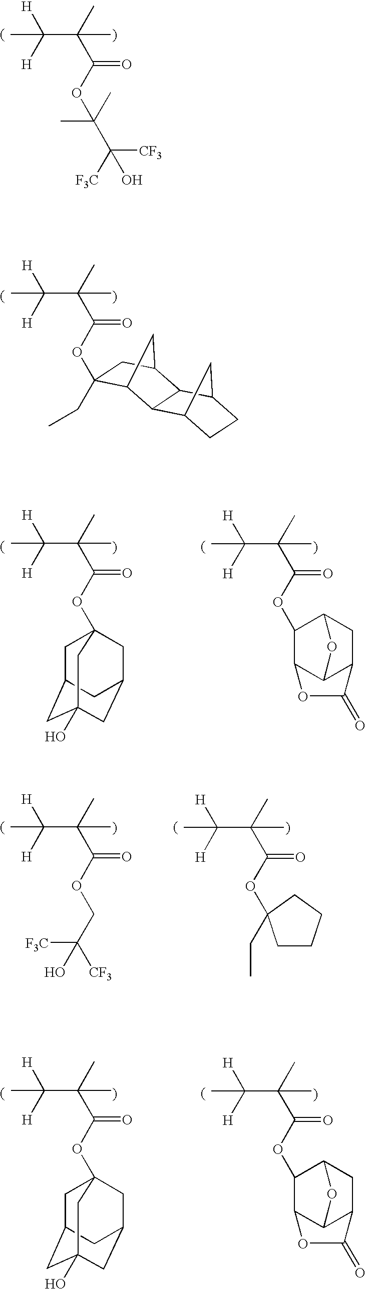

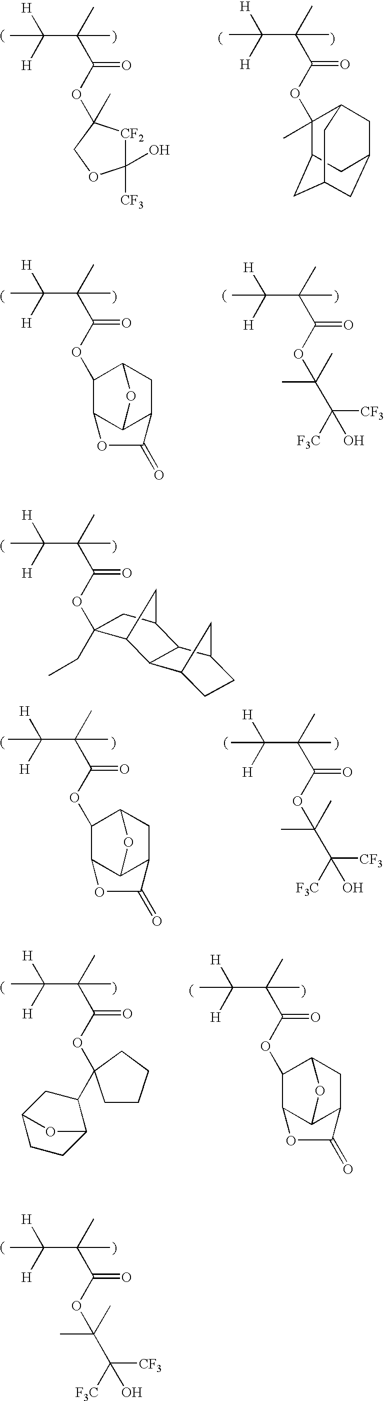

Exemplary polymers comprising recurring units incorporated at compositional ratios a2′, b2′, c2′, d2′ and e′ in formula (R1) are shown below, but not limited thereto.

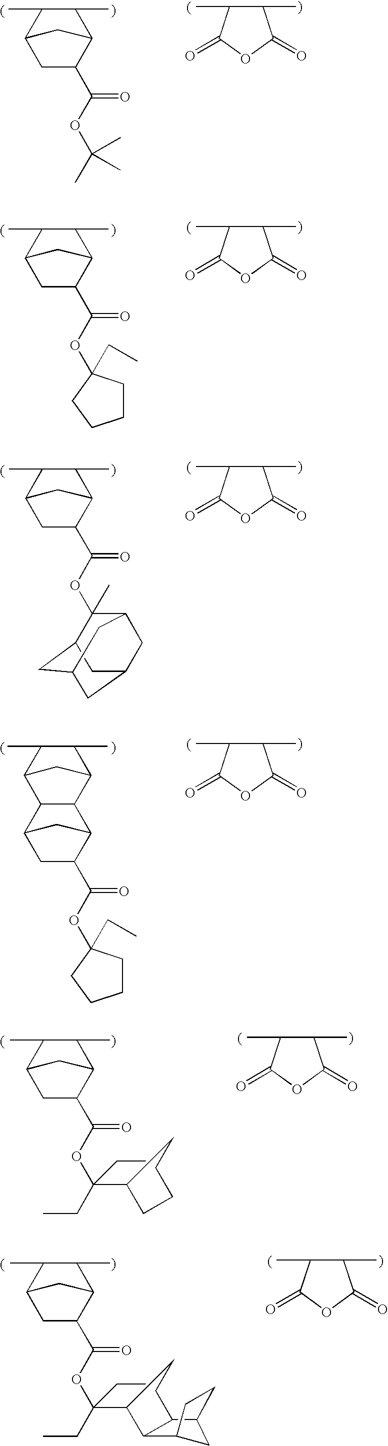

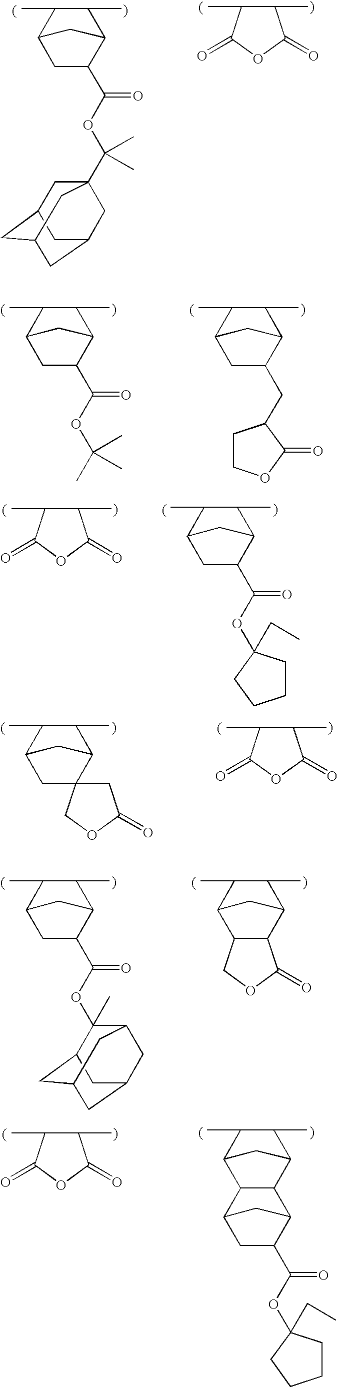

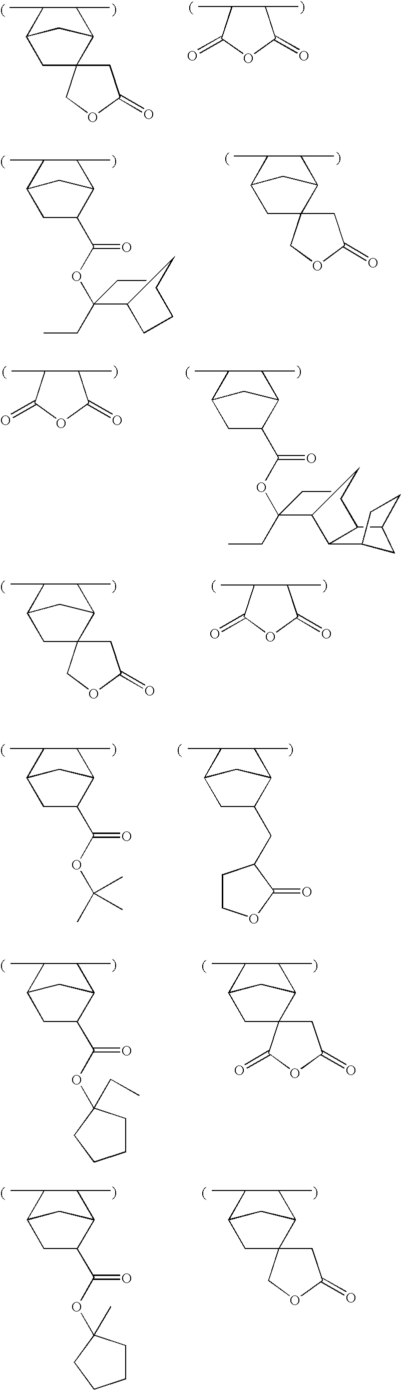

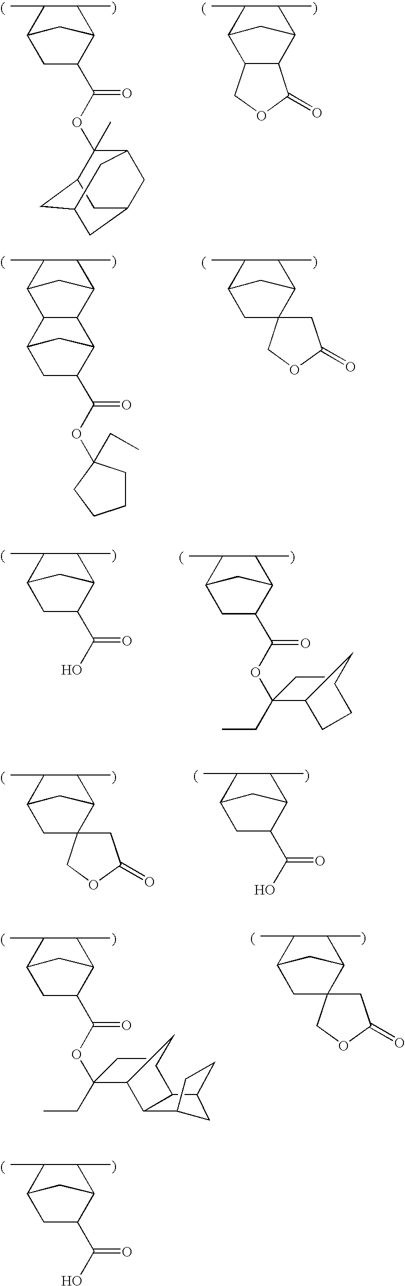

Exemplary polymers comprising recurring units incorporated at compositional ratios a3′, b3′, c3′, and d3′ in formula (R1) are shown below, but not limited thereto.

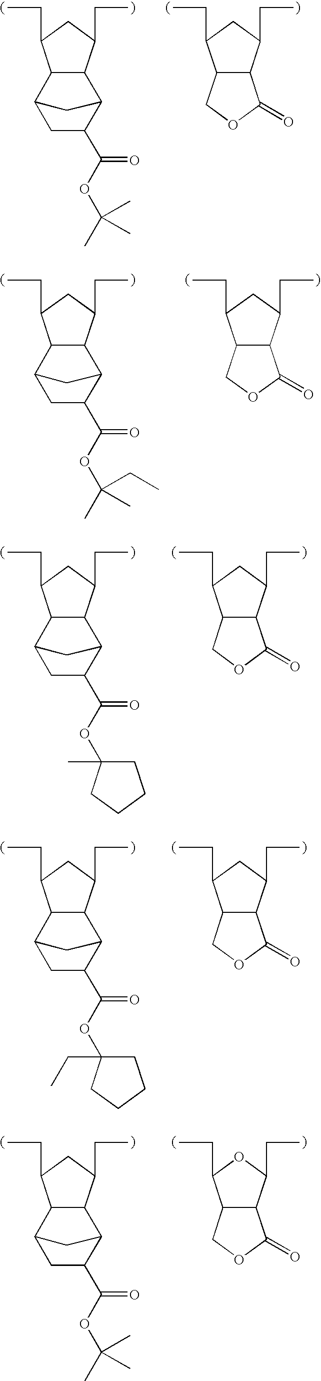

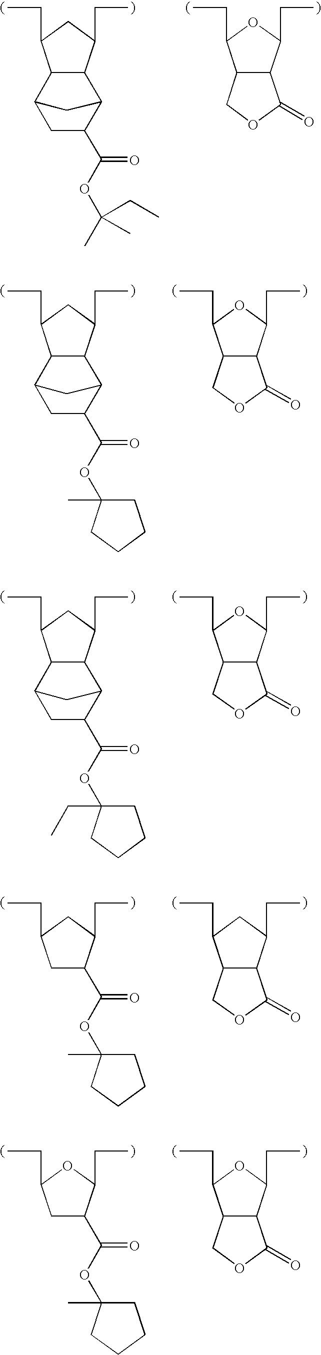

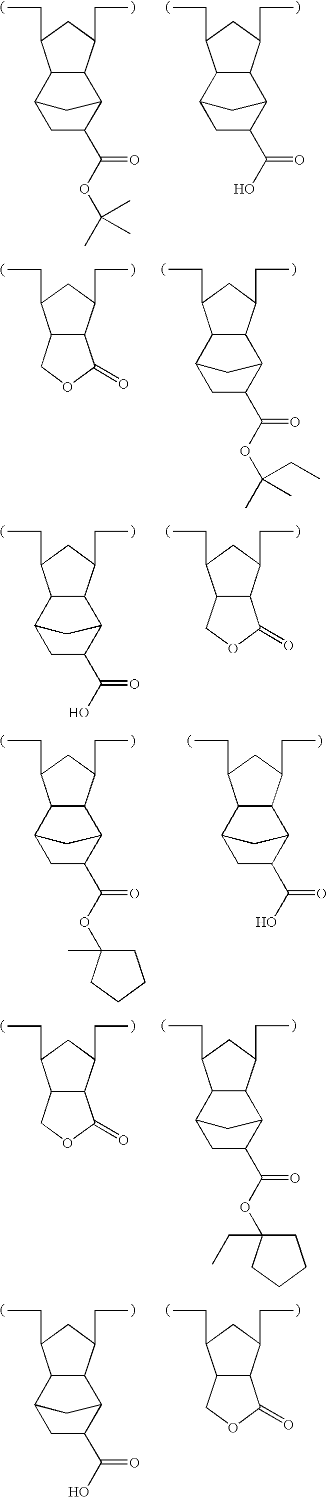

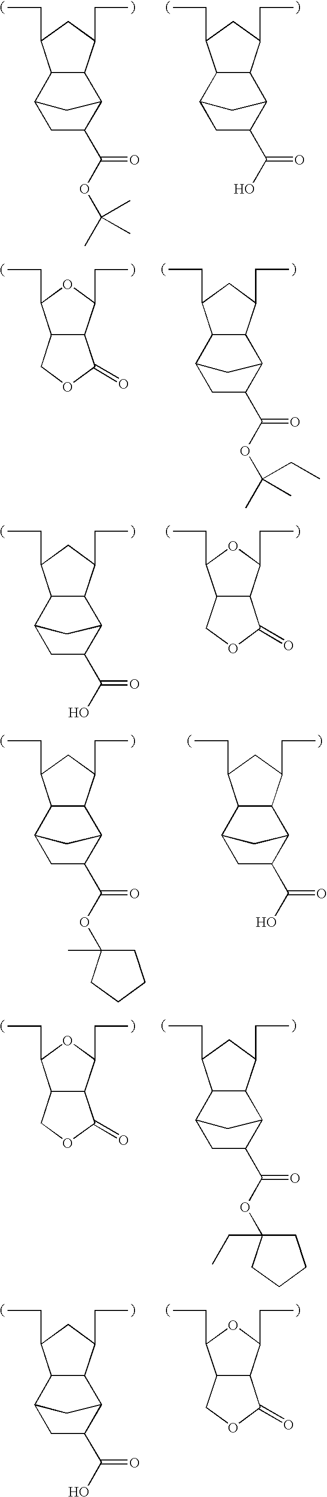

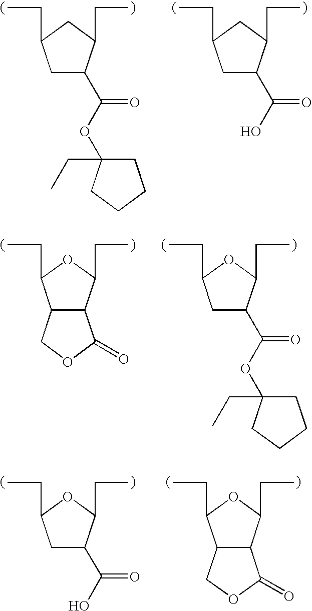

Examples of polymers having formula (R2) are shown below, but not limited thereto.

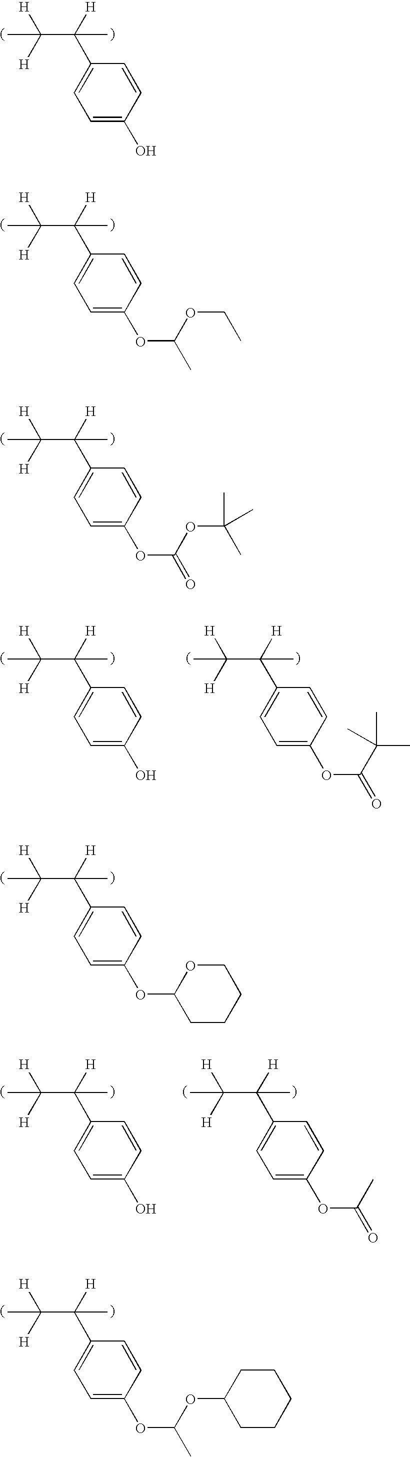

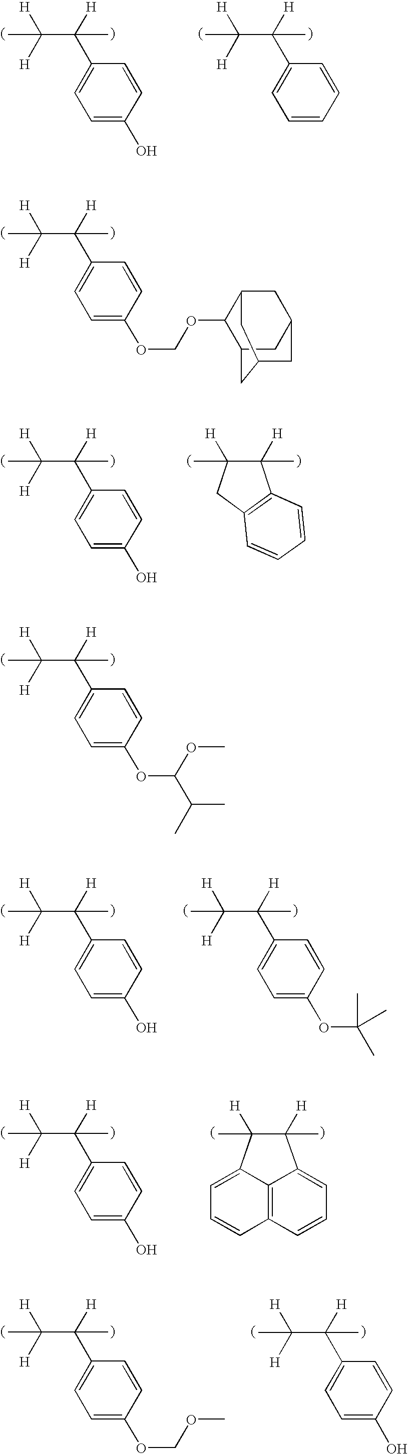

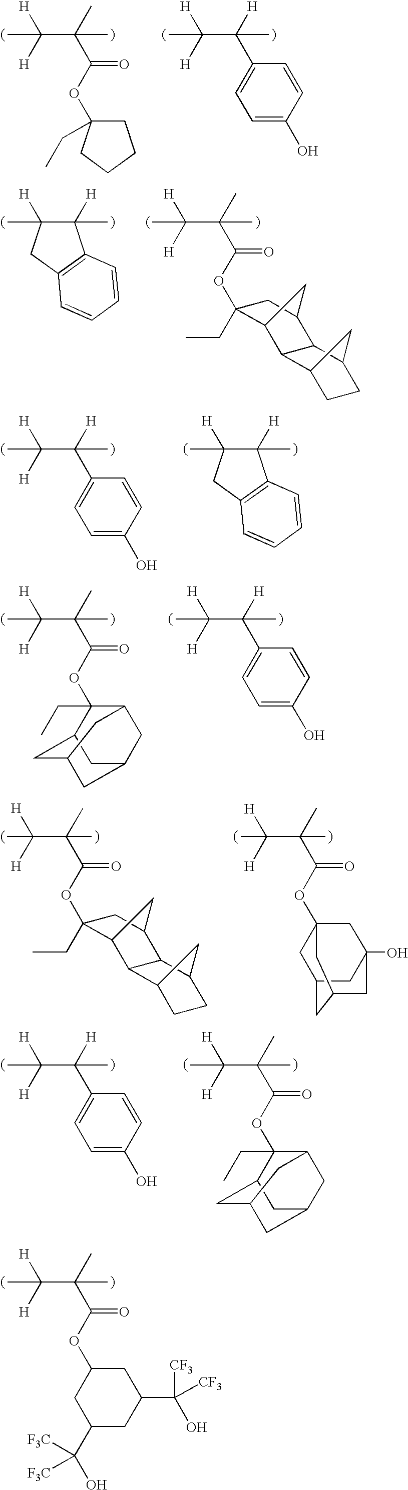

The other polymer is blended in an amount of preferably 0 to 80 parts, more preferably 0 to 60 parts, and even more preferably 0 to 50 parts by weight, provided that the total of the resin component (A) and the other polymer is 100 parts by weight. When blended, the amount of the other polymer is preferably at least 20 parts, more preferably at least 30 parts by weight. Too much amounts of the other polymer may prevent the resin component (A) from exerting its own effect, probably resulting in a lower resolution and degraded pattern profile. The other polymer is not limited to one type and a mixture of two or more other polymers may be added. The use of plural polymers allows for easy adjustment of resist properties.

### Acid Generator

As the compound which generates an acid in response to actinic light or radiation (B), the resist composition of the invention may further comprise (B′) such a compound other than the sulfonium salt compound of formula (4). Component (B′) may be any compound which generates an acid upon exposure to high-energy radiation and specifically, any of well-known photoacid generators which are commonly used in prior art resist compositions, especially chemically amplified resist compositions. Suitable photoacid generators include sulfonium salts, iodonium salts, sulfonyldiazomethane, N-sulfonyloxyimide, and oxime-O-sulfonate acid generators. Exemplary acid generators are given below while they may be used alone or in admixture of two or more.

Sulfonium salts are salts of sulfonium cations with sulfonates, bis(substituted alkylsulfonyl)imides and tris(substituted alkylsulfonyl)methides. Exemplary sulfonium cations include triphenylsulfonium, (4-tert-butoxyphenyl)diphenylsulfonium, bis(4-tert-butoxyphenyl)phenylsulfonium, tris(4-tert-butoxyphenyl)sulfonium, (3-tert-butoxyphenyl)diphenylsulfonium, bis(3-tert-butoxyphenyl)phenylsulfonium, tris(3-tert-butoxyphenyl)sulfonium, (3,4-di-tert-butoxyphenyl)diphenylsulfonium, bis(3,4-di-tert-butoxyphenyl)phenylsulfonium, tris(3,4-di-tert-butoxyphenyl)sulfonium, diphenyl(4-thiophenoxyphenyl)sulfonium, (4-tert-butoxycarbonylmethyloxyphenyl)diphenylsulfonium, tris(4-tert-butoxycarbonylmethyloxyphenyl)sulfonium, (4-tert-butoxyphenyl)bis(4-dimethylaminophenyl)sulfonium, tris(4-dimethylaminophenyl)sulfonium, 4-methylphenyldiphenylsulfonium, 4-tert-butylphenyldiphenylsulfonium, bis(4-methylphenyl)phenylsulfonium, bis(4-tert-butylphenyl)phenylsulfonium, tris(4-methylphenyl)sulfonium, tris(4-tert-butylphenyl)sulfonium, tris(phenylmethyl)sulfonium, 2-naphthyldiphenylsulfonium, dimethyl(2-naphthyl)sulfonium, 4-hydroxyphenyldimethylsulfonium, 4-methoxyphenyldimethylsulfonium, trimethylsulfonium, 2-oxocyclohexylcyclohexylmethylsulfonium, trinaphthylsulfonium, tribenzylsulfonium, diphenylmethylsulfonium, dimethylphenylsulfonium, 2-oxopropylthiacyclopentanium, 2-oxobutylthiacyclopentanium, 2-oxo-3,3-dimethylbutylthiacyclopentanium, 2-oxo-2-phenylethylthiacyclopentanium, 4-n-butoxynaphthyl-1-thiacyclopentanium, and 2-n-butoxynaphthyl-1-thiacyclopentanium. Exemplary sulfonates include trifluoromethanesulfonate, pentafluoroethanesulfonate, heptafluoropropanesulfonate, nonafluorobutanesulfonate, tridecafluorohexanesulfonate, perfluoro(4-ethylcyclohexane)sulfonate, heptadecafluorooctanesulfonate, 2,2,2-trifluoroethanesulfonate, pentafluorobenzenesulfonate, 4-(trifluoromethyl)benzenesulfonate, 4-fluorobenzenesulfonate, mesitylenesulfonate, 2,4,6-triisopropylbenzenesulfonate, toluenesulfonate, benzenesulfonate, 4-(p-toluenesulfonyloxy)benzenesulfonate, 6-(p-toluenesulfonyloxy)naphthalene-2-sulfonate, 4-(p-toluenesulfonyloxy)naphthalene-1-sulfonate, 5-(p-toluenesulfonyloxy)naphthalene-1-sulfonate, 8-(p-toluenesulfonyloxy)naphthalene-1-sulfonate, naphthalenesulfonate, camphorsulfonate, octanesulfonate, dodecylbenzenesulfonate, butanesulfonate, methanesulfonate, 1,1-difluoro-2-naphthyl-ethanesulfonate, 1,1,2,2-tetrafluoro-2-(norbornan-2-yl)ethanesulfonate, 1,1,2,2-tetrafluoro-2-(tetracyclo[6.2.1.1^{3,6} .0^{2,7}]dodec-3-en-8-yl)ethanesulfonate, 2-benzoyloxy-1,1,3,3,3-pentafluoropropanesulfonate, 1,1,3,3,3-pentafluoro-2-(4-phenylbenzoyloxy)propanesulfonate, 1,1,3,3,3-pentafluoro-2-pivaloyloxypropanesulfonate, 2-cyclohexanecarbonyloxy-1,1,3,3,3-pentafluoropropanesulfonate, 1,1,3,3,3-pentafluoro-2-furoyloxypropanesulfonate, 2-naphthoyloxy-1,1,3,3,3-pentafluoropropanesulfonate, 2-(4-tert-butylbenzoyloxy)-1,1,3,3,3-pentafluoropropanesulfonate, 2-(1-adamantanecarbonyloxy)-1,1,3,3,3-pentafluoropropane-sulfonate, 2-acetyloxy-1,1,3,3,3-pentafluoropropanesulfonate, 1,1,3,3,3-pentafluoro-2-hydroxypropanesulfonate, 1,1,3,3,3-pentafluoro-2-tosyloxypropanesulfonate, 1,1-difluoro-2-tosyloxyethanesulfonate, adamantanemethoxycarbonyldifluoromethanesulfonate, 1-(3-hydroxymethyladamantane)methoxycarbonyldifluoromethane-sulfonate, methoxycarbonyldifluoromethanesulfonate, 1-(hexahydro-2-oxo-3,5-methano-2H-cyclopenta[b]furan-6-yloxy-carbonyl)difluoromethanesulfonate, and 4-oxo-1-adamantyloxycarbonyldifluoromethanesulfonate. Exemplary bis(substituted alkylsulfonyl)imides include bis(trifluoromethylsulfonyl)imide, bis(pentafluoroethylsulfonyl)imide, bis(heptafluoropropylsulfonyl)imide, and perfluoro(1,3-propylenebissulfonyl)imide. A typical tris(substituted alkylsulfonyl)methide is tris(trifluoromethylsulfonyl)methide. Sulfonium salts based on combination of the foregoing examples are included.

Iodonium salts are salts of iodonium cations with sulfonates, bis(substituted alkylsulfonyl)imides and tris(substituted alkylsulfonyl)methides. Exemplary iodonium cations are aryliodonium cations including diphenyliodinium, bis(4-tert-butylphenyl)iodonium, 4-tert-butoxyphenylphenyliodonium, and 4-methoxyphenylphenyliodonium. Exemplary sulfonates include trifluoromethanesulfonate, pentafluoroethanesulfonate, heptafluoropropanesulfonate, nonafluorobutanesulfonate, tridecafluorohexanesulfonate, perfluoro(4-ethylcyclohexane)sulfonate, heptadecafluorooctanesulfonate, 2,2,2-trifluoroethanesulfonate, pentafluorobenzenesulfonate, 4-(trifluoromethyl)benzenesulfonate, 4-fluorobenzenesulfonate, mesitylenesulfonate, 2,4,6-triisopropylbenzenesulfonate, toluenesulfonate, benzenesulfonate, 4-(p-toluenesulfonyloxy)benzenesulfonate, 6-(p-toluenesulfonyloxy)naphthalene-2-sulfonate, 4-(p-toluenesulfonyloxy)naphthalene-1-sulfonate, 5-(p-toluenesulfonyloxy)naphthalene-1-sulfonate, 8-(p-toluenesulfonyloxy)naphthalene-1-sulfonate, naphthalenesulfonate, camphorsulfonate, octanesulfonate, dodecylbenzenesulfonate, butanesulfonate, methanesulfonate, 1,1-difluoro-2-naphthyl-ethanesulfonate, 1,1,2,2-tetrafluoro-2-(norbornan-2-yl)ethanesulfonate, 1,1,2,2-tetrafluoro-2-(tetracyclo[6.2.1.1^{3,6}.0^{2,7}]dodec-3-en-8-yl)ethanesulfonate, 2-benzoyloxy-1,1,3,3,3-pentafluoropropanesulfonate, 1,1,3,3,3-pentafluoro-2-(4-phenylbenzoyloxy)propanesulfonate, 1,1,3,3,3-pentafluoro-2-pivaloyloxypropanesulfonate, 2-cyclohexanecarbonyloxy-1,1,3,3,3-pentafluoropropanesulfonate, 1,1,3,3,3-pentafluoro-2-furoyloxypropanesulfonate, 2-naphthoyloxy-1,1,3,3,3-pentafluoropropanesulfonate, 2-(4-tert-butylbenzoyloxy)-1,1,3,3,3-pentafluoropropane-sulfonate, 2-(1-adamantanecarbonyloxy)-1,1,3,3,3-pentafluoropropane-sulfonate, 2-acetyloxy-1,1,3,3,3-pentafluoropropanesulfonate, 1,1,3,3,3-pentafluoro-2-hydroxypropanesulfonate, 1,1,3,3,3-pentafluoro-2-tosyloxypropanesulfonate, 1,1-difluoro-2-tosyloxyethanesulfonate, adamantanemethoxycarbonyldifluoromethanesulfonate, 1-(3-hydroxymethyladamantane)methoxycarbonyldifluoromethane-sulfonate, methoxycarbonyldifluoromethanesulfonate, 1-(hexahydro-2-oxo-3,5-methano-2H-cyclopenta[b]furan-6-yloxy-carbonyl)difluoromethanesulfonate, and 4-oxo-1-adamantyloxycarbonyldifluoromethanesulfonate. Exemplary bis(substituted alkylsulfonyl)imides include bis(trifluoromethylsulfonyl)imide, bis(pentafluoroethylsulfonyl)imide, bis(heptafluoropropylsulfonyl)imide, and perfluoro(1,3-propylenebissulfonyl)imide. A typical tris(substituted alkylsulfonyl)methide is tris(trifluoromethylsulfonyl)methide. Iodonium salts based on combination of the foregoing examples are included.

Exemplary sulfonyldiazomethane compounds include bissulfonyldiazomethane compounds and sulfonylcarbonyldiazomethane compounds such as bis(ethylsulfonyl)diazomethane, bis(1-methylpropylsulfonyl)diazomethane, bis(2-methylpropylsulfonyl)diazomethane, bis(1,1-dimethylethylsulfonyl)diazomethane, bis(cyclohexylsulfonyl)diazomethane, bis(perfluoroisopropylsulfonyl)diazomethane, bis(phenylsulfonyl)diazomethane, bis(4-methylphenylsulfonyl)diazomethane, bis(2,4-dimethylphenylsulfonyl)diazomethane, bis(2-naphthylsulfonyl)diazomethane, bis(4-acetyloxyphenylsulfonyl)diazomethane, bis(4-methanesulfonyloxyphenylsulfonyl)diazomethane, bis(4-(4-toluenesulfonyloxy)phenylsulfonyl)diazomethane, bis(4-(n-hexyloxy)phenylsulfonyl)diazomethane, bis(2-methyl-4-(n-hexyloxy)phenylsulfonyl)diazomethane, bis(2,5-dimethyl-4-(n-hexyloxy)phenylsulfonyl)diazomethane, bis(3,5-dimethyl-4-(n-hexyloxy)phenylsulfonyl)diazomethane, bis(2-methyl-5-isopropyl-4-(n-hexyloxy)phenylsulfonyl)diazo-methane, 4-methylphenylsulfonylbenzoyldiazomethane, tert-butylcarbonyl-4-methylphenylsulfonyldiazomethane, 2-naphthylsulfonylbenzoyldiazomethane, 4-methylphenylsulfonyl-2-naphthoyldiazomethane, methylsulfonylbenzoyldiazomethane, and tert-butoxycarbonyl-4-methylphenylsulfonyldiazomethane.

N-sulfonyloxyimide photoacid generators include combinations of imide structures with sulfonates. Exemplary imide structures are succinimide, naphthalene dicarboxylic acid imide, phthalimide, cyclohexyldicarboxylic acid imide, 5-norbornene-2,3-dicarboxylic acid imide, and 7-oxabicyclo[2.2.1]-5-heptene-2,3-dicarboxylic acid imide. Exemplary sulfonates include trifluoromethanesulfonate, pentafluoroethanesulfonate, nonafluorobutanesulfonate, dodecafluorohexanesulfonate, pentafluoroethylperfluorocyclohexanesulfonate, heptadecafluorooctanesulfonate, 2,2,2-trifluoroethanesulfonate, pentafluorobenzenesulfonate, 4-trifluoromethylbenzenesulfonate, 4-fluorobenzenesulfonate, mesitylenesulfonate, 2,4,6-triisopropylbenzenesulfonate, toluenesulfonate, benzenesulfonate, naphthalenesulfonate, camphorsulfonate, octanesulfonate, dodecylbenzenesulfonate, butanesulfonate, methanesulfonate, 2-benzoyloxy-1,1,3,3,3-pentafluoropropanesulfonate, 1,1,3,3,3-pentafluoro-2-(4-phenylbenzoyloxy)propanesulfonate, 1,1,3,3,3-pentafluoro-2-pivaloyloxypropanesulfonate, 2-cyclohexanecarbonyloxy-1,1,3,3,3-pentafluoropropanesulfonate, 1,1,3,3,3-pentafluoro-2-furoyloxypropanesulfonate, 2-naphthoyloxy-1,1,3,3,3-pentafluoropropanesulfonate, 2-(4-tert-butylbenzoyloxy)-1,1,3,3,3-pentafluoropropane-sulfonate, 2-adamantanecarbonyloxy-1,1,3,3,3-pentafluoropropanesulfonate, 2-acetyloxy-1,1,3,3,3-pentafluoropropanesulfonate, 1,1,3,3,3-pentafluoro-2-hydroxypropanesulfonate, 1,1,3,3,3-pentafluoro-2-tosyloxypropanesulfonate, 1,1-difluoro-2-naphthyl-ethanesulfonate, 1,1,2,2-tetrafluoro-2-(norbornan-2-yl)ethanesulfonate, and 1,1,2,2-tetrafluoro-2-(tetracyclo[4.4.0.1^{2,5}.1^{7,10}]dodec-3-en-8-yl)ethanesulfonate.

Benzoinsulfonate photoacid generators include benzoin tosylate, benzoin mesylate, and benzoin butanesulfonate.

Pyrogallol trisulfonate photoacid generators include pyrogallol, phloroglucinol, catechol, resorcinol, and hydroquinone, in which all the hydroxyl groups are substituted by trifluoromethanesulfonate, pentafluoroethanesulfonate, nonafluorobutanesulfonate, dodecafluorohexanesulfonate, pentafluoroethylperfluorocyclohexanesulfonate, heptadecafluorooctanesulfonate, 2,2,2-trifluoroethanesulfonate, pentafluorobenzenesulfonate, 4-trifluoromethylbenzenesulfonate, 4-fluorobenzenesulfonate, toluenesulfonate, benzenesulfonate, naphthalenesulfonate, camphorsulfonate, octanesulfonate, dodecylbenzenesulfonate, butanesulfonate, methanesulfonate, 2-benzoyloxy-1,1,3,3,3-pentafluoropropanesulfonate, 1,1,3,3,3-pentafluoro-2-(4-phenylbenzoyloxy)propanesulfonate, 1,1,3,3,3-pentafluoro-2-pivaloyloxypropanesulfonate, 2-cyclohexanecarbonyloxy-1,1,3,3,3-pentafluoropropanesulfonate, 1,1,3,3,3-pentafluoro-2-furoyloxypropanesulfonate, 2-naphthoyloxy-1,1,3,3,3-pentafluoropropanesulfonate, 2-(4-tert-butylbenzoyloxy)-1,1,3,3,3-pentafluoropropane-sulfonate, 2-adamantanecarbonyloxy-1,1,3,3,3-pentafluoropropanesulfonate, 2-acetyloxy-1,1,3,3,3-pentafluoropropanesulfonate, 1,1,3,3,3-pentafluoro-2-hydroxypropanesulfonate, 1,1,3,3,3-pentafluoro-2-tosyloxypropanesulfonate, 1,1-difluoro-2-naphthyl-ethanesulfonate, 1,1,2,2-tetrafluoro-2-(norbornan-2-yl)ethanesulfonate, and 1,1,2,2-tetrafluoro-2-(tetracyclo[4.4.0.1^{2,5}.1^{7,10}]dodec-3-en-8-yl)ethanesulfonate.

Nitrobenzyl sulfonate photoacid generators include 2,4-dinitrobenzyl sulfonates, 2-nitrobenzyl sulfonates, and 2,6-dinitrobenzyl sulfonates, with exemplary sulfonates including trifluoromethanesulfonate, pentafluoroethanesulfonate, nonafluorobutanesulfonate, dodecafluorohexanesulfonate, pentafluoroethylperfluorocyclohexanesulfonate, heptadecafluorooctanesulfonate, 2,2,2-trifluoroethanesulfonate, pentafluorobenzenesulfonate, 4-trifluoromethylbenzenesulfonate, 4-fluorobenzenesulfonate, toluenesulfonate, benzenesulfonate, naphthalenesulfonate, camphorsulfonate, octanesulfonate, dodecylbenzenesulfonate, butanesulfonate, methanesulfonate, 2-benzoyloxy-1,1,3,3,3-pentafluoropropanesulfonate, 1,1,3,3,3-pentafluoro-2-(4-phenylbenzoyloxy)propanesulfonate, 1,1,3,3,3-pentafluoro-2-pivaloyloxypropanesulfonate, 2-cyclohexanecarbonyloxy-1,1,3,3,3-pentafluoropropanesulfonate, 1,1,3,3,3-pentafluoro-2-furoyloxypropanesulfonate, 2-naphthoyloxy-1,1,3,3,3-pentafluoropropanesulfonate, 2-(4-tert-butylbenzoyloxy)-1,1,3,3,3-pentafluoropropane-sulfonate, 2-adamantanecarbonyloxy-1,1,3,3,3-pentafluoropropanesulfonate, 2-acetyloxy-1,1,3,3,3-pentafluoropropanesulfonate, 1,1,3,3,3-pentafluoro-2-hydroxypropanesulfonate, 1,1,3,3,3-pentafluoro-2-tosyloxypropanesulfonate, 1,1-difluoro-2-naphthyl-ethanesulfonate, 1,1,2,2-tetrafluoro-2-(norbornan-2-yl)ethanesulfonate, and 1,1,2,2-tetrafluoro-2-(tetracyclo[4.4.0.1^{2,5}.1^{7,10}]dodec-3-en-8-yl)ethanesulfonate. Also useful are analogous nitrobenzyl sulfonate compounds in which the nitro group on the benzyl side is substituted by a trifluoromethyl group.

Sulfone photoacid generators include
bis(phenylsulfonyl)methane,
bis(4-methylphenylsulfonyl)methane,
bis(2-naphthylsulfonyl)methane,
2,2-bis(phenylsulfonyl)propane,
2,2-bis(4-methylphenylsulfonyl)propane,
2,2-bis(2-naphthylsulfonyl)propane,
2-methyl-2-(p-toluenesulfonyl)propiophenone,
2-cyclohexylcarbonyl-2-(p-toluenesulfonyl)propane, and
2,4-dimethyl-2-(p-toluenesulfonyl)pentan-3-one.

Photoacid generators in the form of glyoxime derivatives are described in JP 2906999 and JP-A 9-301948 and include bis-O-(p-toluenesulfonyl)-α-dimethylglyoxime, bis-O-(p-toluenesulfonyl)-α-diphenylglyoxime, bis-O-(p-toluenesulfonyl)-α-dicyclohexylglyoxime, bis-O-(p-toluenesulfonyl)-2,3-pentanedioneglyoxime, bis-O-(n-butanesulfonyl)-α-dimethylglyoxime, bis-O-(n-butanesulfonyl)-α-diphenylglyoxime, bis-O-(n-butanesulfonyl)-α-dicyclohexylglyoxime, bis-O-(methanesulfonyl)-α-dimethylglyoxime, bis-O-(trifluoromethanesulfonyl)-α-dimethylglyoxime, bis-O-(2,2,2-trifluoroethanesulfonyl)-α-dimethylglyoxime, bis-O-(10-camphorsulfonyl)-α-dimethylglyoxime, bis-O-(benzenesulfonyl)-α-dimethylglyoxime, bis-O-(p-fluorobenzenesulfonyl)-α-dimethylglyoxime, bis-O-(p-trifluoromethylbenzenesulfonyl)-α-dimethylglyoxime, bis-O-(xylenesulfonyl)-α-dimethylglyoxime, bis-O-(trifluoromethanesulfonyl)-nioxime, bis-O-(2,2,2-trifluoroethanesulfonyl)-nioxime, bis-O-(10-camphorsulfonyl)-nioxime, bis-O-(benzenesulfonyl)-nioxime, bis-O-(p-fluorobenzenesulfonyl)-nioxime, bis-O-(p-trifluoromethylbenzenesulfonyl)-nioxime, and bis-O-(xylenesulfonyl)-nioxime.

Also included are the oxime sulfonates described in U.S. Pat. No. 6,004,724, for example,
(5-(4-toluenesulfonyl)oxyimino-5H-thiophen-2-ylidene)phenyl-acetonitrile,
(5-(10-camphorsulfonyl)oxyimino-5H-thiophen-2-ylidene)phenyl-acetonitrile,
(5-n-octanesulfonyloxyimino-5H-thiophen-2-ylidene)phenyl-acetonitrile,
(5-(4-toluenesulfonyl)oxyimino-5H-thiophen-2-ylidene)(2-methylphenyl)acetonitrile,
(5-(10-camphorsulfonyl)oxyimino-5H-thiophen-2-ylidene)(2-methylphenyl)acetonitrile,
(5-n-octanesulfonyloxyimino-5H-thiophen-2-ylidene)(2-methyl-phenyl)acetonitrile, etc. Also included are the oxime sulfonates described in U.S. Pat. No. 6,916,591, for example,
(5-(4-(4-toluenesulfonyloxy)benzenesulfonyl)oxyimino-5H-thiophen-2-ylidene)phenylacetonitrile and
(5-(2,5-bis(4-toluenesulfonyloxy)benzenesulfonyl)oxyimino-5H-thiophen-2-ylidene)phenylacetonitrile.

Also included are the oxime sulfonates described in U.S. Pat. No. 6,261,738 and JP-A 2000-314956, for example, 2,2,2-trifluoro-1-phenyl-ethanone oxime-O-methylsulfonate; 2,2,2-trifluoro-1-phenyl-ethanone oxime-O-(10-camphoryl-sulfonate); 2,2,2-trifluoro-1-phenyl-ethanone oxime-O-(4-methoxyphenylsulfonate); 2,2,2-trifluoro-1-phenyl-ethanone oxime-O-(1-naphthylsulfonate); 2,2,2-trifluoro-1-phenyl-ethanone oxime-O-(2-naphthylsulfonate); 2,2,2-trifluoro-1-phenyl-ethanone oxime-O-(2,4,6-trimethylphenylsulfonate); 2,2,2-trifluoro-1-(4-methylphenyl)-ethanone oxime-O-(10-camphorylsulfonate); 2,2,2-trifluoro-1-(4-methylphenyl)-ethanone oxime-O-(methylsulfonate); 2,2,2-trifluoro-1-(2-methylphenyl)-ethanone oxime-O-(10-camphorylsulfonate); 2,2,2-trifluoro-1-(2,4-dimethylphenyl)-ethanone oxime-O-(10-camphorylsulfonate); 2,2,2-trifluoro-1-(2,4-dimethylphenyl)-ethanone oxime-O-(1-naphthylsulfonate); 2,2,2-trifluoro-1-(2,4-dimethylphenyl)-ethanone oxime-O-(2-naphthylsulfonate); 2,2,2-trifluoro-1-(2,4,6-trimethylphenyl)-ethanone oxime-O-(10-camphorylsulfonate); 2,2,2-trifluoro-1-(2,4,6-trimethyl-phenyl)-ethanone oxime-O-(1-naphthylsulfonate); 2,2,2-trifluoro-1-(2,4,6-trimethylphenyl)-ethanone oxime-O-(2-naphthylsulfonate); 2,2,2-trifluoro-1-(4-methoxyphenyl)-ethanone oxime-O-methylsulfonate; 2,2,2-trifluoro-1-(4-methylthiophenyl)-ethanone oxime-O-methylsulfonate; 2,2,2-trifluoro-1-(3,4-dimethoxyphenyl)-ethanone oxime-O-methylsulfonate; 2,2,3,3,4,4,4-heptafluoro-1-phenyl-butanone oxime-O-(10-camphorylsulfonate); 2,2,2-trifluoro-1-(phenyl)-ethanone oxime-O-methylsulfonate; 2,2,2-trifluoro-1-(phenyl)-ethanone oxime-O-10-camphorylsulfonate; 2,2,2-trifluoro-1-(phenyl)-ethanone oxime-O-(4-methoxyphenyl)sulfonate; 2,2,2-trifluoro-1-(phenyl)-ethanone oxime-O-(1-naphthyl)-sulfonate; 2,2,2-trifluoro-1-(phenyl)-ethanone oxime-O-(2-naphthyl)sulfonate; 2,2,2-trifluoro-1-(phenyl)-ethanone oxime-O-(2,4,6-trimethylphenyl)sulfonate; 2,2,2-trifluoro-1-(4-methylphenyl)-ethanone oxime-O-(10-camphoryl)sulfonate; 2,2,2-trifluoro-1-(4-methylphenyl)-ethanone oxime-O-methyl-sulfonate; 2,2,2-trifluoro-1-(2-methylphenyl)-ethanone oxime-O-(10-camphoryl)sulfonate; 2,2,2-trifluoro-1-(2,4-dimethyl-phenyl)-ethanone oxime-O-(1-naphthyl)sulfonate; 2,2,2-trifluoro-1-(2,4-dimethylphenyl)-ethanone oxime-O-(2-naphthyl)sulfonate; 2,2,2-trifluoro-1-(2,4,6-trimethyl-phenyl)-ethanone oxime-O-(10-camphoryl)sulfonate; 2,2,2-trifluoro-1-(2,4,6-trimethylphenyl)-ethanone oxime-O-(1-naphthyl)sulfonate; 2,2,2-trifluoro-1-(2,4,6-trimethyl-phenyl)-ethanone oxime-O-(2-naphthyl)sulfonate; 2,2,2-trifluoro-1-(4-methoxyphenyl)-ethanone oxime-O-methyl-sulfonate; 2,2,2-trifluoro-1-(4-thiomethylphenyl)-ethanone oxime-O-methylsulfonate; 2,2,2-trifluoro-1-(3,4-dimethoxy-phenyl)-ethanone oxime-O-methylsulfonate; 2,2,2-trifluoro-1-(4-methoxyphenyl)-ethanone oxime-O-(4-methylphenyl)sulfonate; 2,2,2-trifluoro-1-(4-methoxyphenyl)-ethanone oxime-O-(4-methoxyphenyl)sulfonate; 2,2,2-trifluoro-1-(4-methoxyphenyl)-ethanone oxime-O-(4-dodecylphenyl)sulfonate; 2,2,2-trifluoro-1-(4-methoxyphenyl)-ethanone oxime-O-octylsulfonate; 2,2,2-trifluoro-1-(4-thiomethylphenyl)-ethanone oxime-O-(4-methoxyphenyl)sulfonate; 2,2,2-trifluoro-1-(4-thiomethyl-phenyl)-ethanone oxime-O-(4-dodecylphenyl)sulfonate; 2,2,2-trifluoro-1-(4-thiomethylphenyl)-ethanone oxime-O-octylsulfonate; 2,2,2-trifluoro-1-(4-thiomethylphenyl)-ethanone oxime-O-(2-naphthyl)sulfonate; 2,2,2-trifluoro-1-(2-methylphenyl)-ethanone oxime-O-methylsulfonate; 2,2,2-trifluoro-1-(4-methylphenyl)ethanone oxime-O-phenylsulfonate; 2,2,2-trifluoro-1-(4-chlorophenyl)-ethanone oxime-O-phenyl-sulfonate; 2,2,3,3,4,4,4-heptafluoro-1-(phenyl)-butanone oxime-O-(10-camphoryl)sulfonate; 2,2,2-trifluoro-1-naphthyl-ethanone oxime-O-methylsulfonate; 2,2,2-trifluoro-2-naphthyl-ethanone oxime-O-methylsulfonate; 2,2,2-trifluoro-1-[4-benzylphenyl]-ethanone oxime-O-methylsulfonate; 2,2,2-trifluoro-1-[4-(phenyl-1,4-dioxa-but-1-yl)phenyl]-ethanone oxime-O-methylsulfonate; 2,2,2-trifluoro-1-naphthyl-ethanone oxime-O-propylsulfonate; 2,2,2-trifluoro-2-naphthyl-ethanone oxime-O-propylsulfonate; 2,2,2-trifluoro-1-[4-benzylphenyl]-ethanone oxime-O-propylsulfonate; 2,2,2-trifluoro-1-[4-methylsulfonylphenyl]-ethanone oxime-O-propylsulfonate; 1,3-bis[1-(4-phenoxyphenyl)-2,2,2-trifluoro-ethanone oxime-O-sulfonyl]phenyl; 2,2,2-trifluoro-1-[4-methylsulfonyloxyphenyl]-ethanone oxime-O-propylsulfonate; 2,2,2-trifluoro-1-[4-methylcarbonyloxyphenyl]-ethanone oxime-O-propylsulfonate; 2,2,2-trifluoro-1-[6H,7H-5,8-dioxonaphth-2-yl]-ethanone oxime-O-propylsulfonate; 2,2,2-trifluoro-1-[4-methoxycarbonylmethoxyphenyl]-ethanone oxime-O-propyl-sulfonate; 2,2,2-trifluoro-1-[4-(methoxycarbonyl)-(4-amino-1-oxa-pent-1-yl)-phenyl]-ethanone oxime-O-propylsulfonate; 2,2,2-trifluoro-1-[3,5-dimethyl-4-ethoxyphenyl]-ethanone oxime-O-propylsulfonate; 2,2,2-trifluoro-1-[4-benzyloxy-phenyl]-ethanone oxime-O-propylsulfonate; 2,2,2-trifluoro-1-[2-thiophenyl]-ethanone oxime-O-propylsulfonate; 2,2,2-trifluoro-1-[1-dioxa-thiophen-2-yl)]-ethanone oxime-O-propylsulfonate; 2,2,2-trifluoro-1-(4-(3-(4-(2,2,2-trifluoro-1-(trifluoromethanesulfonyloxyimino)-ethyl)-phenoxy)-propoxy)-phenyl)ethanone oxime(trifluoromethanesulfonate); 2,2,2-trifluoro-1-(4-(3-(4-(2,2,2-trifluoro-1-(1-propane-sulfonyloxyimino)-ethyl)-phenoxy)-propoxy)-phenyl)ethanone oxime(1-propanesulfonate); and 2,2,2-trifluoro-1-(4-(3-(4-(2,2,2-trifluoro-1-(1-butanesulfonyloxyimino)-ethyl)-phenoxy)-propoxy)-phenyl)ethanone oxime(1-butanesulfonate). Also included are the oxime sulfonates described in U.S. Pat. No. 6,916,591, for example, 2,2,2-trifluoro-1-(4-(3-(4-(2,2,2-trifluoro-1-(4-(4-methylphenylsulfonyloxy)phenylsulfonyloxy-imino)-ethyl)-phenoxy)-propoxy)-phenyl)ethanone oxime(4-(4-methylphenylsulfonyloxy)phenylsulfonate) and 2,2,2-trifluoro-1-(4-(3-(4-(2,2,2-trifluoro-1-(2,5-bis(4-methylphenyl-sulfonyloxy) phenylsulfonyloxyimino)-ethyl)-phenoxy)-propoxy)-phenyl)ethanone oxime(2,5-bis(4-methylphenylsulfonyloxy)-phenylsulfonate).

Also included are the oxime sulfonates described in JP-A 9-95479 and JP-A 9-230588 and the references cited therein, for example,
α-(p-toluenesulfonyloxyimino)-phenylacetonitrile,
α-(p-chlorobenzenesulfonyloxyimino)-phenylacetonitrile,
α-(4-nitrobenzenesulfonyloxyimino)-phenylacetonitrile,
α-(4-nitro-2-trifluoromethylbenzenesulfonyloxyimino)-phenylacetonitrile,
α-(benzenesulfonyloxyimino)-4-chlorophenylacetonitrile,
α-(benzenesulfonyloxyimino)-2,4-dichlorophenylacetonitrile,
α-(benzenesulfonyloxyimino)-2,6-dichlorophenylacetonitrile,
α-(benzenesulfonyloxyimino)-4-methoxyphenylacetonitrile,
α-(2-chlorobenzenesulfonyloxyimino)-4-methoxyphenylaceto-nitrile,
α-(benzenesulfonyloxyimino)-2-thienylacetonitrile,
α-(4-dodecylbenzenesulfonyloxyimino)-phenylacetonitrile,
α[(4-toluenesulfonyloxyimino)-4-methoxyphenyl]acetonitrile,
α[(dodecylbenzenesulfonyloxyimino)-4-methoxyphenyl]aceto-nitrile,
α-(tosyloxyimino)-3-thienylacetonitrile,
α-(methylsulfonyloxyimino)-1-cyclopentenylacetonitrile,
α-(ethylsulfonyloxyimino)-1-cyclopentenylacetonitrile,
α-(isopropylsulfonyloxyimino)-1-cyclopentenylacetonitrile,
α-(n-butylsulfonyloxyimino)-1-cyclopentenylacetonitrile,
α-(ethylsulfonyloxyimino)-1-cyclohexenylacetonitrile,
α-(isopropylsulfonyloxyimino)-1-cyclohexenylacetonitrile, and
α-(n-butylsulfonyloxyimino)-1-cyclohexenylacetonitrile.

Also included are oxime sulfonates having the following formula, examples of which are described, for example, in WO 2004/074242.

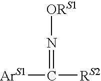

Herein R^{s1} is a substituted or unsubstituted haloalkylsulfonyl or halobenzenesulfonyl group of 1 to 10 carbon atoms, R^{s2} is a haloalkyl group of 1 to 11 carbon atoms, and Ar^{s1} is substituted or unsubstituted aromatic or hetero-aromatic group.

Examples Include
2-[2,2,3,3,4,4,5,5-octafluoro-1-(nonafluorobutylsulfonyloxy-imino)-pentyl]-fluorene,
2-[2,2,3,3,4,4-pentafluoro-1-(nonafluorobutylsulfonyloxy-imino)-butyl]-fluorene,
2-[2,2,3,3,4,4,5,5,6,6-decafluoro-1-(nonafluorobutylsulfonyl-oxyimino)-hexyl]-fluorene,
2-[2,2,3,3,4,4,5,5-octafluoro-1-(nonafluorobutylsulfonyloxy-imino)-pentyl]-4-biphenyl,
2-[2,2,3,3,4,4-pentafluoro-1-(nonafluorobutylsulfonyloxy-imino)-butyl]-4-biphenyl, and
2-[2,2,3,3,4,4,5,5,6,6-decafluoro-1-(nonafluorobutylsulfonyl-oxyimino)-hexyl]-4-biphenyl.

Suitable bisoxime sulfonates include those described in JP-A 9-208554, for example,
bis(α-(4-toluenesulfonyloxy)imino)-p-phenylenediacetonitrile,
bis(α-(benzenesulfonyloxy)imino)-p-phenylenediacetonitrile,
bis(α-(methanesulfonyloxy)imino)-p-phenylenediacetonitrile,
bis(α-(butanesulfonyloxy)imino)-p-phenylenediacetonitrile,
bis(α-(10-camphorsulfonyloxy)imino)-p-phenylenediacetonitrile,
bis(α-(4-toluenesulfonyloxy)imino)-p-phenylenediacetonitrile,
bis(α-(trifluoromethanesulfonyloxy)imino)-p-phenylenediaceto-nitrile,
bis(α-(4-methoxybenzenesulfonyloxy)imino)-p-phenylenediaceto-nitrile,
bis(α-(4-toluenesulfonyloxy)imino)-m-phenylenediacetonitrile,
bis(α-(benzenesulfonyloxy)imino)-m-phenylenediacetonitrile,
bis(α-(methanesulfonyloxy)imino)-m-phenylenediacetonitrile,
bis(α-(butanesulfonyloxy)imino)-m-phenylenediacetonitrile,
bis(α-(10-camphorsulfonyloxy)imino)-m-phenylenediacetonitrile,
bis(α-(4-toluenesulfonyloxy)imino)-m-phenylenediacetonitrile,
bis(α-(trifluoromethanesulfonyloxy)imino)-m-phenylenediaceto-nitrile,
bis(α-(4-methoxybenzenesulfonyloxy)imino)-m-phenylenediaceto-nitrile, etc.

Of these, preferred photoacid generators are sulfonium salts, bissulfonyldiazomethanes, N-sulfonyloxyimides, oxime-O-sulfonates and glyoxime derivatives. More preferred photoacid generators are sulfonium salts, bissulfonyldiazomethanes, N-sulfonyloxyimides, and oxime-O-sulfonates. Typical examples include
triphenylsulfonium p-toluenesulfonate,
triphenylsulfonium camphorsulfonate,
triphenylsulfonium pentafluorobenzenesulfonate,
triphenylsulfonium nonafluorobutanesulfonate,
triphenylsulfonium 4-(4′-toluenesulfonyloxy)benzenesulfonate,
triphenylsulfonium 2,4,6-triisopropylbenzenesulfonate,
4-tert-butoxyphenyldiphenylsulfonium p-toluenesulfonate,
4-tert-butoxyphenyldiphenylsulfonium camphorsulfonate,
4-tert-butoxyphenyldiphenylsulfonium 4-(4′-toluenesulfonyl-oxy)benzenesulfonate,
tris(4-methylphenyl)sulfonium camphorsulfonate,
tris(4-tert-butylphenyl)sulfonium camphorsulfonate,
4-tert-butylphenyldiphenylsulfonium camphorsulfonate,
4-tert-butylphenyldiphenylsulfonium nonafluoro-1-butane-sulfonate,
4-tert-butylphenyldiphenylsulfonium pentafluoroethyl-perfluorocyclohexanesulfonate,
4-tert-butylphenyldiphenylsulfonium perfluoro-1-octane-sulfonate,
triphenylsulfonium 1,1-difluoro-2-naphthyl-ethanesulfonate,
triphenylsulfonium 1,1,2,2-tetrafluoro-2-(norbornan-2-yl)-ethanesulfonate,
bis(tert-butylsulfonyl)diazomethane,
bis(cyclohexylsulfonyl)diazomethane,
bis(2,4-dimethylphenylsulfonyl)diazomethane,
bis(4-(n-hexyloxy)phenylsulfonyl)diazomethane,
bis(2-methyl-4-(n-hexyloxy)phenylsulfonyl)diazomethane,
bis(2,5-dimethyl-4-(n-hexyloxy)phenylsulfonyl)diazomethane,
bis(3,5-dimethyl-4-(n-hexyloxy)phenylsulfonyl)diazomethane,
bis(2-methyl-5-isopropyl-4-(n-hexyloxy)phenylsulfonyl)diazo-methane,
bis(4-tert-butylphenylsulfonyl)diazomethane,
N-camphorsulfonyloxy-5-norbornene-2,3-dicarboxylic acid imide,
N-p-toluenesulfonyloxy-5-norbornene-2,3-dicarboxylic acid imide,
2-[2,2,3,3,4,4,5,5-octafluoro-1-(nonafluorobutylsulfonyloxy-imino)-pentyl]-fluorene,
2-[2,2,3,3,4,4-pentafluoro-1-(nonafluorobutylsulfonyloxy-imino)-butyl]-fluorene, and
2-[2,2,3,3,4,4,5,5,6,6-decafluoro-1-(nonafluorobutylsulfonyl-oxyimino)-hexyl]-fluorene.

In the chemically amplified resist composition, an appropriate amount of the photoacid generators (B) and (B′) is, but not limited to, 0.1 to 40 parts, and especially 0.1 to 20 parts by weight per 100 parts by weight of the base polymer (i.e., resin component (A) and optional other resin component). Too high a proportion of the photoacid generators may give rise to problems of degraded resolution and foreign matter upon development and resist film peeling. Provided that [B] and [B′] stand for the amounts of generators (B) and (B′) added, respectively, the preferred blending proportion of generators (B) and (B′) is 0.1≦[B]/([B]+[B′])≦1, more preferably 0.3≦[B]/([B]+[B′])≦1, and even more preferably 0.5≦[B]/([B]+[B′])≦1. If a blending proportion of generator (B) is too low, then exposure dose dependency, pattern density dependency and/or mask fidelity may be degraded. The photoacid generators (B) and (B′) each may be used alone or in admixture of two or more. The transmittance of the resist film can be controlled by using a photoacid generator having a low transmittance at the exposure wavelength and adjusting the amount of the photoacid generator added.

In the resist composition, there may be added a compound which is decomposed with an acid to generate another acid, that is, acid-amplifier compound. For these compounds, reference should be made to J. Photopolym. Sci. and Tech., 8, 43-44, 45-46 (1995), and ibid., 9, 29-30 (1996).

Examples of the acid-amplifier compound include tert-butyl-2-methyl-2-tosyloxymethyl acetoacetate and 2-phenyl-2-(2-tosyloxyethyl)-1,3-dioxolane, but are not limited thereto. Of well-known photoacid generators, many of those compounds having poor stability, especially poor thermal stability exhibit an acid amplifier-like behavior.

In the resist composition, an appropriate amount of the acid-amplifier compound is 0 to 2 parts, and especially 0 to 1 part by weight per 100 parts by weight of the base polymer. Excessive amounts of the acid-amplifier compound make diffusion control difficult, leading to degradation of resolution and pattern profile.

In addition to components (A) and (B), the resist composition may further comprise (C) an organic solvent and optionally (D) an organic nitrogen-containing compound, (E) a surfactant, and (F) other components.

### Organic Solvent

The organic solvent (C) used herein may be any organic solvent in which the base resin, acid generator, and additives are soluble. Illustrative, non-limiting, examples of the organic solvent include ketones such as cyclohexanone and methyl amyl ketone; alcohols such as 3-methoxybutanol, 3-methyl-3-methoxybutanol, 1-methoxy-2-propanol, and 1-ethoxy-2-propanol; ethers such as propylene glycol monomethyl ether, ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as γ-butyrolactone. These solvents may be used alone or in combinations of two or more thereof. Of the above organic solvents, it is recommended to use diethylene glycol dimethyl ether, 1-ethoxy-2-propanol, propylene glycol monomethyl ether acetate, and mixtures thereof because the acid generator is most soluble therein.

An appropriate amount of the organic solvent used is 200 to 3,000 parts, especially 400 to 2,500 parts by weight per 100 parts by weight of the base polymer.

### Nitrogen-Containing Compound

In the resist composition, an organic nitrogen-containing compound or compounds may be compounded as component (D). The organic nitrogen-containing compound used herein is preferably a compound capable of suppressing the rate of diffusion when the acid generated by the acid generator diffuses within the resist film. The inclusion of organic nitrogen-containing compound holds down the rate of acid diffusion within the resist film, resulting in better resolution. In addition, it suppresses changes in sensitivity following exposure and reduces substrate and environment dependence, as well as improving the exposure latitude and the pattern profile.

The organic nitrogen-containing compound used herein may be any of well-known organic nitrogen-containing compounds which are commonly used in prior art resist compositions, especially chemically amplified resist compositions. Suitable organic nitrogen-containing compounds include primary, secondary, and tertiary aliphatic amines, mixed amines, aromatic amines, heterocyclic amines, nitrogen-containing compounds having carboxyl group, nitrogen-containing compounds having sulfonyl group, nitrogen-containing compounds having hydroxyl group, nitrogen-containing compounds having hydroxyphenyl group, alcoholic nitrogen-containing compounds, amide derivatives, imide derivatives, carbamate derivatives, and ammonium salts.

Examples of suitable primary aliphatic amines include ammonia, methylamine, ethylamine, n-propylamine, isopropylamine, n-butylamine, isobutylamine, sec-butylamine, tert-butylamine, pentylamine, tert-amylamine, cyclopentylamine, hexylamine, cyclohexylamine, heptylamine, octylamine, nonylamine, decylamine, dodecylamine, cetylamine, methylenediamine, ethylenediamine, and tetraethylenepentamine. Examples of suitable secondary aliphatic amines include dimethylamine, diethylamine, di-n-propylamine, diisopropylamine, di-n-butylamine, diisobutylamine, di-sec-butylamine, dipentylamine, dicyclopentylamine, dihexylamine, dicyclohexylamine, diheptylamine, dioctylamine, dinonylamine, didecylamine, didodecylamine, dicetylamine, N,N-dimethylmethylenediamine, N,N-dimethylethylenediamine, and N,N-dimethyltetraethylenepentamine. Examples of suitable tertiary aliphatic amines include trimethylamine, triethylamine, tri-n-propylamine, triisopropylamine, tri-n-butylamine, triisobutylamine, tri-sec-butylamine, tripentylamine, tricyclopentylamine, trihexylamine, tricyclohexylamine, triheptylamine, trioctylamine, trinonylamine, tridecylamine, tridodecylamine, tricetylamine, N,N,N′,N′-tetramethylmethylenediamine, N,N,N′,N′-tetramethylethylenediamine, and N,N,N′,N′-tetramethyltetraethylenepentamine.

Examples of suitable mixed amines include dimethylethylamine, methylethylpropylamine, benzylamine, phenethylamine, and benzyldimethylamine. Examples of suitable aromatic and heterocyclic amines include aniline derivatives (e.g., aniline, N-methylaniline, N-ethylaniline, N-propylaniline, N,N-dimethylaniline, 2-methylaniline, 3-methylaniline, 4-methylaniline, ethylaniline, propylaniline, trimethylaniline, 2-nitroaniline, 3-nitroaniline, 4-nitroaniline, 2,4-dinitroaniline, 2,6-dinitroaniline, 3,5-dinitroaniline, and N,N-dimethyltoluidine), diphenyl(p-tolyl)amine, methyldiphenylamine, triphenylamine, phenylenediamine, naphthylamine, diaminonaphthalene, pyrrole derivatives (e.g., pyrrole, 2H-pyrrole, 1-methylpyrrole, 2,4-dimethylpyrrole, 2,5-dimethylpyrrole, and N-methylpyrrole), oxazole derivatives (e.g., oxazole and isooxazole), thiazole derivatives (e.g., thiazole and isothiazole), imidazole derivatives (e.g., imidazole, 4-methylimidazole, and 4-methyl-2-phenylimidazole), pyrazole derivatives, furazan derivatives, pyrroline derivatives (e.g., pyrroline and 2-methyl-1-pyrroline), pyrrolidine derivatives (e.g., pyrrolidine, N-methylpyrrolidine, pyrrolidinone, and N-methylpyrrolidone), imidazoline derivatives, imidazolidine derivatives, pyridine derivatives (e.g., pyridine, methylpyridine, ethylpyridine, propylpyridine, butylpyridine, 4-(1-butylpentyl)pyridine, dimethylpyridine, trimethylpyridine, triethylpyridine, phenylpyridine, 3-methyl-2-phenylpyridine, 4-tert-butylpyridine, diphenylpyridine, benzylpyridine, methoxypyridine, butoxypyridine, dimethoxypyridine, 4-pyrrolidinopyridine, 2-(1-ethylpropyl)pyridine, aminopyridine, and dimethylaminopyridine), pyridazine derivatives, pyrimidine derivatives, pyrazine derivatives, pyrazoline derivatives, pyrazolidine derivatives, piperidine derivatives, piperazine derivatives, morpholine derivatives, indole derivatives, isoindole derivatives, 1H-indazole derivatives, indoline derivatives, quinoline derivatives (e.g., quinoline and 3-quinolinecarbonitrile), isoquinoline derivatives, cinnoline derivatives, quinazoline derivatives, quinoxaline derivatives, phthalazine derivatives, purine derivatives, pteridine derivatives, carbazole derivatives, phenanthridine derivatives, acridine derivatives, phenazine derivatives, 1,10-phenanthroline derivatives, adenine derivatives, adenosine derivatives, guanine derivatives, guanosine derivatives, uracil derivatives, and uridine derivatives.

Examples of suitable nitrogen-containing compounds having carboxyl group include aminobenzoic acid, indolecarboxylic acid, and amino acid derivatives (e.g. nicotinic acid, alanine, alginine, aspartic acid, glutamic acid, glycine, histidine, isoleucine, glycylleucine, leucine, methionine, phenylalanine, threonine, lysine, 3-aminopyrazine-2-carboxylic acid, and methoxyalanine). Examples of suitable nitrogen-containing compounds having sulfonyl group include 3-pyridinesulfonic acid and pyridinium p-toluenesulfonate. Examples of suitable nitrogen-containing compounds having hydroxyl group, nitrogen-containing compounds having hydroxyphenyl group, and alcoholic nitrogen-containing compounds include 2-hydroxypyridine, aminocresol, 2,4-quinolinediol, 3-indolemethanol hydrate, monoethanolamine, diethanolamine, triethanolamine, N-ethyldiethanolamine, N,N-diethylethanolamine, triisopropanolamine, 2,2′-iminodiethanol, 2-aminoethanol, 3-amino-1-propanol, 4-amino-1-butanol, 4-(2-hydroxyethyl)morpholine, 2-(2-hydroxyethyl)pyridine, 1-(2-hydroxyethyl)piperazine, 1-[2-(2-hydroxyethoxy)ethyl]piperazine, piperidine ethanol, 1-(2-hydroxyethyl)pyrrolidine, 1-(2-hydroxyethyl)-2-pyrrolidinone, 3-piperidino-1,2-propanediol, 3-pyrrolidino-1,2-propanediol, 8-hydroxyjulolidine, 3-quinuclidinol, 3-tropanol, 1-methyl-2-pyrrolidine ethanol, 1-aziridine ethanol, N-(2-hydroxyethyl)phthalimide, and N-(2-hydroxyethyl)isonicotinamide. Examples of suitable amide derivatives include formamide, N-methylformamide, N,N-dimethylformamide, acetamide, N-methylacetamide, N,N-dimethylacetamide, propionamide, benzamide, and 1-cyclohexylpyrrolidone. Suitable imide derivatives include phthalimide, succinimide, and maleimide. Suitable carbamate derivatives include N-t-butoxycarbonyl-N,N-dicyclohexylamine, N-t-butoxycarbonylbenzimidazole and oxazolidinone.

Suitable ammonium salts include
pyridinium p-toluenesulfonate,
triethylammonium p-toluenesulfonate,
trioctylammonium p-toluenesulfonate,
triethylammonium 2,4,6-triisopropylbenzenesulfonate,
trioctylammonium 2,4,6-triisopropylbenzenesulfonate,
triethylammonium camphorsulfonate,
trioctylammonium camphorsulfonate,
tetramethylammonium hydroxide, tetraethylammonium hydroxide,
tetrabutylammonium hydroxide,
benzyltrimethylammonium hydroxide,
tetramethylammonium p-toluenesulfonate,
tetrabutylammonium p-toluenesulfonate,
benzyltrimethylammonium p-toluenesulfonate,
tetramethylammonium camphorsulfonate,
tetrabutylammonium camphorsulfonate,
benzyltrimethylammonium camphorsulfonate,
tetramethylammonium 2,4,6-triisopropylbenzenesulfonate,
tetrabutylammonium 2,4,6-triisopropylbenzenesulfonate,
benzyltrimethylammonium 2,4,6-triisopropylbenzenesulfonate,
tetramethylammonium acetate, tetrabutylammonium acetate,
benzyltrimethylammonium acetate, tetramethylammonium benzoate,
tetrabutylammonium benzoate, and
benzyltrimethylammonium benzoate.

Also included are organic nitrogen-containing compounds of the following general formula (B)-1.

N(X)ₙ(Y)₃₋ₙ (B)-1

In the formula, n is equal to 1, 2 or 3; side chain Y is independently hydrogen or a straight, branched or cyclic C₁-C₂₀ alkyl group which may contain an ether or hydroxyl group; and side chain X is independently selected from groups of the following general formulas (X1) to (X3), and two or three X's may bond together to form a ring.

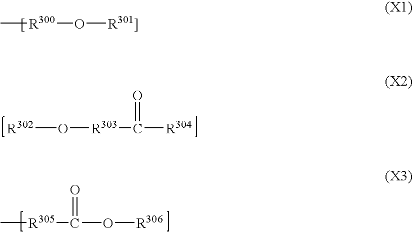

In the formulas, R³⁰⁰, R³⁰² and R³⁰⁵ are independently straight or branched C₁-C₄ alkylene groups; R³⁰¹ and R³⁰⁴ are independently hydrogen, or straight, branched or cyclic C₁-C₂₀ alkyl groups which may contain at least one hydroxyl, ether, ester group or lactone ring; R³⁰³ is a single bond or a straight or branched C₁-C₂₀ alkylene group; and R³⁰⁶ is a straight, branched or cyclic C₁-C₂₀ alkyl group which may contain at least one hydroxyl, ether, ester group or lactone ring.

Illustrative examples of the compounds of formula
(B)-1 include tris(2-methoxymethoxyethyl)amine,
tris{2-(2-methoxyethoxy)ethyl}amine,
tris{2-(2-methoxyethoxymethoxy)ethyl}amine,
tris{2-(1-methoxyethoxy)ethyl}amine,
tris{2-(1-ethoxyethoxy)ethyl}amine,
tris{2-(1-ethoxypropoxy)ethyl}amine,
tris[2-{2-(2-hydroxyethoxy)ethoxy}ethyl]amine,
4,7,13,16,21,24-hexaoxa-1,10-diazabicyclo[8.8.8]hexacosane,
4,7,13,18-tetraoxa-1,10-diazabicyclo[8.5.5]eicosane,
1,4,10,13-tetraoxa-7,16-diazabicyclooctadecane,
1-aza-12-crown-4, 1-aza-15-crown-5, 1-aza-18-crown-6,
tris(2-formyloxyethyl)amine, tris(2-acetoxyethyl)amine,
tris(2-propionyloxyethyl)amine, tris(2-butyryloxyethyl)amine,
tris(2-isobutyryloxyethyl)amine, tris(2-valeryloxyethyl)amine,
tris(2-pivaloyloxyethyl)amine,
N,N-bis(2-acetoxyethyl)-2-(acetoxyacetoxy)ethylamine,
tris(2-methoxycarbonyloxyethyl)amine,
tris(2-tert-butoxycarbonyloxyethyl)amine,
tris[2-(2-oxopropoxy)ethyl]amine,
tris[2-(methoxycarbonylmethyl)oxyethyl]amine,
tris[2-(tert-butoxycarbonylmethyloxy)ethyl]amine,
tris[2-(cyclohexyloxycarbonylmethyloxy)ethyl]amine,
tris(2-methoxycarbonylethyl)amine,
tris(2-ethoxycarbonylethyl)amine,
N,N-bis(2-hydroxyethyl)-2-(methoxycarbonyl)ethylamine,
N,N-bis(2-acetoxyethyl)-2-(methoxycarbonyl)ethylamine,
N,N-bis(2-hydroxyethyl)-2-(ethoxycarbonyl)ethylamine,
N,N-bis(2-acetoxyethyl)-2-(ethoxycarbonyl)ethylamine,
N,N-bis(2-hydroxyethyl)-2-(2-methoxyethoxycarbonyl)ethylamine,
N,N-bis(2-acetoxyethyl)-2-(2-methoxyethoxycarbonyl)ethylamine,
N,N-bis(2-hydroxyethyl)-2-(2-hydroxyethoxycarbonyl)ethylamine,
N,N-bis(2-acetoxyethyl)-2-(2-acetoxyethoxycarbonyl)ethylamine,
N,N-bis(2-hydroxyethyl)-2-[(methoxycarbonyl)methoxycarbonyl]-ethylamine,
N,N-bis(2-acetoxyethyl)-2-[(methoxycarbonyl)methoxycarbonyl]-ethylamine,
N,N-bis(2-hydroxyethyl)-2-(2-oxopropoxycarbonyl)ethylamine,
N,N-bis(2-acetoxyethyl)-2-(2-oxopropoxycarbonyl)ethylamine,
N,N-bis(2-hydroxyethyl)-2-(tetrahydrofurfuryloxycarbonyl)-ethylamine,
N,N-bis(2-acetoxyethyl)-2-(tetrahydrofurfuryloxycarbonyl)-ethylamine,
N,N-bis(2-hydroxyethyl)-2-[(2-oxotetrahydrofuran-3-yl)oxy-carbonyl]ethylamine,
N,N-bis(2-acetoxyethyl)-2-[(2-oxotetrahydrofuran-3-yl)oxy-carbonyl]ethylamine,
N,N-bis(2-hydroxyethyl)-2-(4-hydroxybutoxycarbonyl)ethylamine,
N,N-bis(2-formyloxyethyl)-2-(4-formyloxybutoxycarbonyl)-ethylamine,
N,N-bis(2-formyloxyethyl)-2-(2-formyloxyethoxycarbonyl)-ethylamine,
N,N-bis(2-methoxyethyl)-2-(methoxycarbonyl)ethylamine,
N-(2-hydroxyethyl)-bis[2-(methoxycarbonyl)ethyl]amine,
N-(2-acetoxyethyl)-bis[2-(methoxycarbonyl)ethyl]amine,
N-(2-hydroxyethyl)-bis[2-(ethoxycarbonyl)ethyl]amine,
N-(2-acetoxyethyl)-bis[2-(ethoxycarbonyl)ethyl]amine,
N-(3-hydroxy-1-propyl)-bis[2-(methoxycarbonyl)ethyl]amine,
N-(3-acetoxy-1-propyl)-bis[2-(methoxycarbonyl)ethyl]amine,
N-(2-methoxyethyl)-bis[2-(methoxycarbonyl)ethyl]amine,
N-butyl-bis[2-(methoxycarbonyl)ethyl]amine,
N-butyl-bis[2-(2-methoxyethoxycarbonyl)ethyl]amine,
N-methyl-bis(2-acetoxyethyl)amine,
N-ethyl-bis(2-acetoxyethyl)amine,
N-methyl-bis(2-pivaloyloxyethyl)amine,
N-ethyl-bis[2-(methoxycarbonyloxy)ethyl]amine,
N-ethyl-bis[2-(tert-butoxycarbonyloxy)ethyl]amine,
tris(methoxycarbonylmethyl)amine,
tris(ethoxycarbonylmethyl)amine,
N-butyl-bis(methoxycarbonylmethyl)amine,
N-hexyl-bis(methoxycarbonylmethyl)amine, and
β-(diethylamino)-δ-valerolactone.

Also useful are one or more organic nitrogen-containing compounds having cyclic structure represented by the following general formula (B)-2.

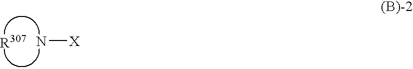

Herein X is as defined above, and R³⁰⁷ is a straight or branched C₂-C₂₀ alkylene group which may contain one or more carbonyl, ether, ester or sulfide groups.

Illustrative examples of the organic nitrogen-containing compounds having formula (B)-2 include
1-[2-(methoxymethoxy)ethyl]pyrrolidine,
1-[2-(methoxymethoxy)ethyl]piperidine,
4-[2-(methoxymethoxy)ethyl]morpholine,
1-[2-[(2-methoxyethoxy)methoxy]ethyl]pyrrolidine,
1-[2-[(2-methoxyethoxy)methoxy]ethyl]piperidine,
4-[2-[(2-methoxyethoxy)methoxy]ethyl]morpholine,
2-(1-pyrrolidinyl)ethyl acetate, 2-piperidinoethyl acetate,
2-morpholinoethyl acetate, 2-(1-pyrrolidinyl)ethyl formate,
2-piperidinoethyl propionate,
2-morpholinoethyl acetoxyacetate,
2-(1-pyrrolidinyl)ethyl methoxyacetate,
4-[2-(methoxycarbonyloxy)ethyl]morpholine,
1-[2-(t-butoxycarbonyloxy)ethyl]piperidine,
4-[2-(2-methoxyethoxycarbonyloxy)ethyl]morpholine,
methyl 3-(1-pyrrolidinyl)propionate,
methyl 3-piperidinopropionate, methyl 3-morpholinopropionate,
methyl 3-(thiomorpholino)propionate,
methyl 2-methyl-3-(1-pyrrolidinyl)propionate,
ethyl 3-morpholinopropionate,
methoxycarbonylmethyl 3-piperidinopropionate,
2-hydroxyethyl 3-(1-pyrrolidinyl)propionate,
2-acetoxyethyl 3-morpholinopropionate,
2-oxotetrahydrofuran-3-yl 3-(1-pyrrolidinyl)propionate,
tetrahydrofurfuryl 3-morpholinopropionate,
glycidyl 3-piperidinopropionate,
2-methoxyethyl 3-morpholinopropionate,
2-(2-methoxyethoxy)ethyl 3-(1-pyrrolidinyl)propionate,
butyl 3-morpholinopropionate,
cyclohexyl 3-piperidinopropionate,
α(1-pyrrolidinyl)methyl-γ-butyrolactone,
β-piperidino-γ-butyrolactone, β-morpholino-δ-valerolactone,
methyl 1-pyrrolidinylacetate, methyl piperidinoacetate,
methyl morpholinoacetate, methyl thiomorpholinoacetate,
ethyl 1-pyrrolidinylacetate, 2-methoxyethyl morpholinoacetate,
2-morpholinoethyl 2-methoxyacetate,
2-morpholinoethyl 2-(2-methoxyethoxy)acetate,
2-morpholinoethyl 2-[2-(2-methoxyethoxy)ethoxy]acetate,
2-morpholinoethyl hexanoate, 2-morpholinoethyl octanoate,
2-morpholinoethyl decanoate, 2-morpholinoethyl laurate,
2-morpholinoethyl myristate, 2-morpholinoethyl palmitate, and
2-morpholinoethyl stearate.

Also, one or more organic nitrogen-containing compounds having cyano group represented by the following general formulae (B)-3 to (B)-6 may be blended.

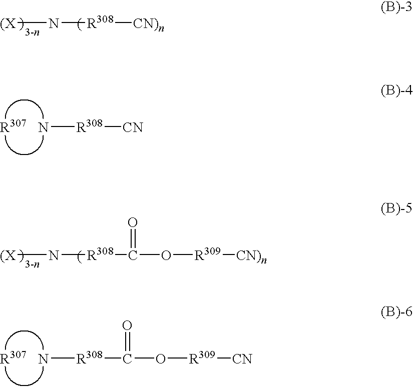

Herein, X, R³⁰⁷ and n are as defined above, and R³⁰⁸ and R³⁰⁹ are each independently a straight or branched C₁-C₄ alkylene group.

Illustrative examples of the organic nitrogen-containing compounds having cyano represented by formulae (B)-3 to (B)-6 include
3-(diethylamino)propiononitrile,
N,N-bis(2-hydroxyethyl)-3-aminopropiononitrile,
N,N-bis(2-acetoxyethyl)-3-aminopropiononitrile,
N,N-bis(2-formyloxyethyl)-3-aminopropiononitrile,
N,N-bis(2-methoxyethyl)-3-aminopropiononitrile,
N,N-bis[2-(methoxymethoxy)ethyl]-3-aminopropiononitrile,
methyl N-(2-cyanoethyl)-N-(2-methoxyethyl)-3-aminopropionate,
methyl N-(2-cyanoethyl)-N-(2-hydroxyethyl)-3-aminopropionate,
methyl N-(2-acetoxyethyl)-N-(2-cyanoethyl)-3-aminopropionate,
N-(2-cyanoethyl)-N-ethyl-3-aminopropiononitrile,
N-(2-cyanoethyl)-N-(2-hydroxyethyl)-3-aminopropiononitrile,
N-(2-acetoxyethyl)-N-(2-cyanoethyl)-3-aminopropiononitrile,
N-(2-cyanoethyl)-N-(2-formyloxyethyl)-3-aminopropiononitrile,
N-(2-cyanoethyl)-N-(2-methoxyethyl)-3-aminopropiononitrile,
N-(2-cyanoethyl)-N-[2-(methoxymethoxy)ethyl]-3-aminopropiono-nitrile,
N-(2-cyanoethyl)-N-(3-hydroxy-1-propyl)-3-aminopropiononitrile,
N-(3-acetoxy-1-propyl)-N-(2-cyanoethyl)-3-aminopropiononitrile,
N-(2-cyanoethyl)-N-(3-formyloxy-1-propyl)-3-aminopropiono-nitrile,
N-(2-cyanoethyl)-N-tetrahydrofurfuryl-3-aminopropiononitrile,
N,N-bis(2-cyanoethyl)-3-aminopropiononitrile,
diethylaminoacetonitrile,
N,N-bis(2-hydroxyethyl)aminoacetonitrile,
N,N-bis(2-acetoxyethyl)aminoacetonitrile,
N,N-bis(2-formyloxyethyl)aminoacetonitrile,
N,N-bis(2-methoxyethyl)aminoacetonitrile,
N,N-bis[2-(methoxymethoxy)ethyl]aminoacetonitrile,
methyl N-cyanomethyl-N-(2-methoxyethyl)-3-aminopropionate,
methyl N-cyanomethyl-N-(2-hydroxyethyl)-3-aminopropionate,
methyl N-(2-acetoxyethyl)-N-cyanomethyl-3-aminopropionate,
N-cyanomethyl-N-(2-hydroxyethyl)aminoacetonitrile,
N-(2-acetoxyethyl)-N-(cyanomethyl)aminoacetonitrile,
N-cyanomethyl-N-(2-formyloxyethyl)aminoacetonitrile,
N-cyanomethyl-N-(2-methoxyethyl)aminoacetonitrile,
N-cyanomethyl-N-[2-(methoxymethoxy)ethyl)aminoacetonitrile,
N-cyanomethyl-N-(3-hydroxy-1-propyl)aminoacetonitrile,
N-(3-acetoxy-1-propyl)-N-(cyanomethyl)aminoacetonitrile,
N-cyanomethyl-N-(3-formyloxy-1-propyl)aminoacetonitrile,
N,N-bis(cyanomethyl)aminoacetonitrile,
1-pyrrolidinepropiononitrile, 1-piperidinepropiononitrile,
4-morpholinepropiononitrile, 1-pyrrolidineacetonitrile,
1-piperidineacetonitrile, 4-morpholineacetonitrile,
cyanomethyl 3-diethylaminopropionate,
cyanomethyl N,N-bis(2-hydroxyethyl)-3-aminopropionate,
cyanomethyl N,N-bis(2-acetoxyethyl)-3-aminopropionate,
cyanomethyl N,N-bis(2-formyloxyethyl)-3-aminopropionate,
cyanomethyl N,N-bis(2-methoxyethyl)-3-aminopropionate,
cyanomethyl N,N-bis[2-(methoxymethoxy)ethyl]-3-aminopropionate,
2-cyanoethyl 3-diethylaminopropionate,
2-cyanoethyl N,N-bis(2-hydroxyethyl)-3-aminopropionate,
2-cyanoethyl N,N-bis(2-acetoxyethyl)-3-aminopropionate,
2-cyanoethyl N,N-bis(2-formyloxyethyl)-3-aminopropionate,
2-cyanoethyl N,N-bis(2-methoxyethyl)-3-aminopropionate,
2-cyanoethyl N,N-bis[2-(methoxymethoxy)ethyl]-3-amino-propionate,
cyanomethyl 1-pyrrolidinepropionate,
cyanomethyl 1-piperidinepropionate,
cyanomethyl 4-morpholinepropionate,
2-cyanoethyl 1-pyrrolidinepropionate,
2-cyanoethyl 1-piperidinepropionate, and
2-cyanoethyl 4-morpholinepropionate.

Also included are organic nitrogen-containing compounds of imidazole structure having a polar functional group, represented by the general formula (B)-7.

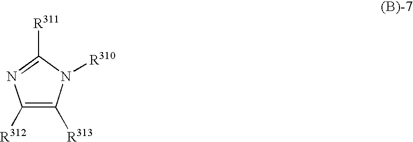

Herein, R³¹⁰ is a straight, branched or cyclic C₂-C₂₀ alkyl group bearing at least one polar functional group selected from among hydroxyl, carbonyl, ester, ether, sulfide, carbonate, cyano and acetal groups; R³¹¹, R³¹² and R³¹³ are each independently a hydrogen atom, a straight, branched or cyclic C₁-C₁₀ alkyl group, aryl group or aralkyl group.

Also included are organic nitrogen-containing compounds of benzimidazole structure having a polar functional group, represented by the general formula (B)-8.

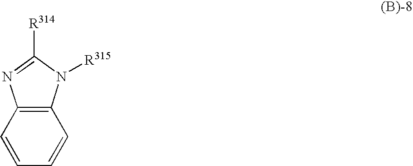

Herein, R³¹⁴ is hydrogen, a straight, branched or cyclic C₁-C₁₀ alkyl group, aryl group or aralkyl group. R³¹⁵ is a polar functional group-bearing, straight, branched or cyclic C₁-C₂₀ alkyl group, and the alkyl group contains as the polar functional group at least one group selected from among ester, acetal and cyano groups, and may additionally contain at least one group selected from among hydroxyl, carbonyl, ether, sulfide and carbonate groups.

Further included are heterocyclic nitrogen-containing compounds having a polar functional group, represented by the general formulae (B)-9 and (B)-10.

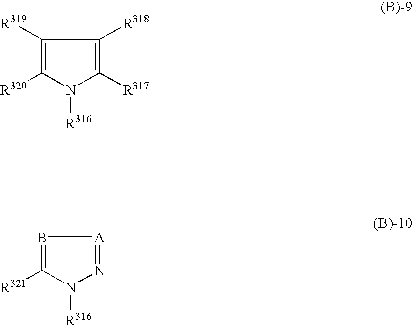

Herein, A is a nitrogen atom or ≡C—R³²², B is a nitrogen atom or ≡C—R³²³, R³¹⁶ is a straight, branched or cyclic C₂-C₂₀ alkyl group bearing at least one polar functional group selected from among hydroxyl, carbonyl, ester, ether, sulfide, carbonate, cyano and acetal groups; R³¹⁷, R³¹⁸, R³¹⁹ and R³²⁰ are each independently hydrogen, a straight, branched or cyclic C₁-C₁₀ alkyl group or aryl group, or a pair of R³¹⁷ and R³¹⁸ or a pair of R³¹⁹ and R³²⁰ may bond together to form a benzene, naphthalene or pyridine ring with the carbon atoms to which they are attached; R³²¹ is hydrogen, a straight, branched or cyclic C₁-C₁₀ alkyl group or aryl group; R³²² and R³²³ each are hydrogen, a straight, branched or cyclic C₁-C₁₀ alkyl group or aryl group, or a pair of R³²¹ and R³²³, taken together, may form a benzene or naphthalene ring with the carbon atom to which they are attached.

Also included are organic nitrogen-containing compounds of aromatic carboxylic ester structure having the general formulae (B)-11 to (B)-14.

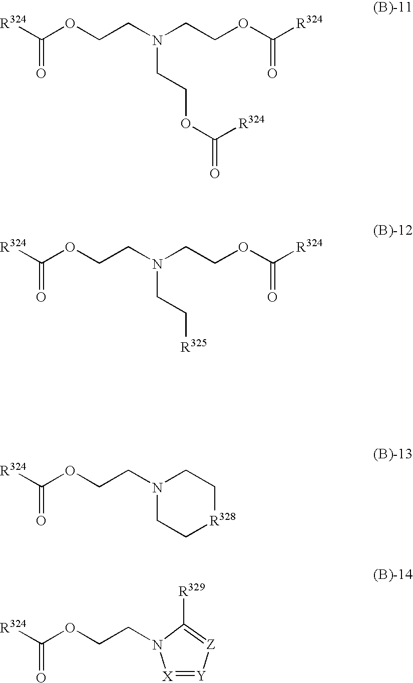

Herein R³²⁴ is a C₆-C₂₀ aryl group or C₄-C₂₀ hetero-aromatic group, in which some or all of hydrogen atoms may be replaced by halogen atoms, straight, branched or cyclic C₁-C₂₀ alkyl groups, C₆-C₂₀ aryl groups, C₇-C₂₀ aralkyl groups, C₁-C₁₀ alkoxy groups, C₁-C₁₀ acyloxy groups or C₁-C₁₀ alkylthio groups. R³²⁵ is CO₂R³²⁶, OR³²⁷ or cyano group. R³²⁶ is a C₁-C₁₀ alkyl group, in which some methylene groups may be replaced by oxygen atoms. R³²⁷ is a C₁-C₁₀ alkyl or acyl group, in which some methylene groups may be replaced by oxygen atoms. R³²⁸ is a single bond, methylene, ethylene, sulfur atom or —O(CH₂CH₂O)ₙ— group wherein n is 0, 1, 2, 3 or 4. R³²⁹ is hydrogen, methyl, ethyl or phenyl. X is a nitrogen atom or CR³³⁰. Y is a nitrogen atom or CR³³¹. Z is a nitrogen atom or CR³³², R³³⁰, R³³¹ and R³³² are each independently hydrogen, methyl or phenyl. Alternatively, a pair of R³³⁰ and R³³¹ or a pair of R³³¹ and R³³² may bond together to form a C₆-C₂₀ aromatic ring or C₂-C₂₀ hetero-aromatic ring with the carbon atoms to which they are attached.

Further included are organic nitrogen-containing compounds of 7-oxanorbornane-2-carboxylic ester structure having the general formula (B)-15.

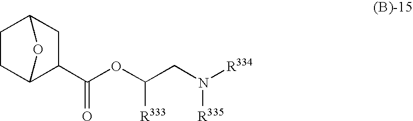

Herein R³³³ is hydrogen or a straight, branched or cyclic C₁-C₁₀ alkyl group. R³³⁴ and R³³⁵ are each independently a C₁-C₂₀ alkyl group, C₆-C₂₀ aryl group or C₇-C₂₀ aralkyl group, which may contain one or more polar functional groups selected from among ether, carbonyl, ester, alcohol, sulfide, nitrile, amine, imine, and amide and in which some hydrogen atoms may be replaced by halogen atoms. R³³⁴ and R³³⁵ may bond together to form a heterocyclic or hetero-aromatic ring of 2 to 20 carbon atoms with the nitrogen atom to which they are attached.

The organic nitrogen-containing compounds may be used alone or in admixture of two or more. The organic nitrogen-containing compound is preferably formulated in an amount of 0.001 to 4 parts, and especially 0.01 to 2 parts by weight, per 100 parts by weight of the base polymer. Less than 0.001 part of the nitrogen-containing compound achieves no or little addition effect whereas more than 4 parts may result in too low a sensitivity.

### Other Components

Optionally, the resist composition of the invention may further comprise (E) a surfactant which is commonly used for facilitating the coating operation. The surfactant may be added in conventional amounts so long as this does not compromise the objects of the invention.

Illustrative, non-limiting examples of the surfactant include nonionic surfactants, for example, polyoxyethylene alkyl ethers such as polyoxyethylene lauryl ether, polyoxyethylene stearyl ether, polyoxyethylene cetyl ether, and polyoxyethylene oleyl ether, polyoxyethylene alkylaryl ethers such as polyoxyethylene octylphenol ether and polyoxyethylene nonylphenol ether, polyoxyethylene polyoxypropylene block copolymers, sorbitan fatty acid esters such as sorbitan monolaurate, sorbitan monopalmitate, and sorbitan monostearate, and polyoxyethylene sorbitan fatty acid esters such as polyoxyethylene sorbitan monolaurate, polyoxyethylene sorbitan monopalmitate, polyoxyethylene sorbitan monostearate, polyoxyethylene sorbitan trioleate, and polyoxyethylene sorbitan tristearate; fluorochemical surfactants such as EFTOP EF301, EF303 and EF352 (JEMCO Inc.), Megaface F171, F172, F173, R08, R30, R90 and R94 (DIC Corp.), Fluorad FC-430, FC-431, FC-4430 and FC-4432 (Sumitomo 3M Co., Ltd.), Asahiguard AG710, Surflon S-381, S-382, S-386, SC101, SC102, SC103, SC104, SC105, SC106, KH-10, KH-20, KH-30 and KH-40 (Asahi Glass Co., Ltd.); organosiloxane polymers KP341, X-70-092 and X-70-093 (Shin-Etsu Chemical Co., Ltd.), acrylic acid or methacrylic acid Polyflow No. 75 and No. 95 (Kyoeisha Ushi Kagaku Kogyo Co., Ltd.). Additional useful surfactants include partially fluorinated oxetane ring-opened polymers having the structural formula (surf-1).

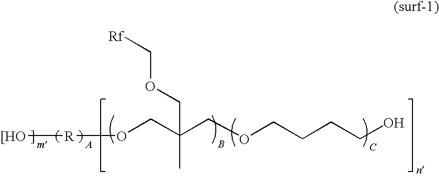

It is provided herein that R, Rf, A, B, C, m′, and n′ are applied to only formula (surf-1), independent of their descriptions other than for the surfactant. R is a di- to tetra-valent C₂-C₅ aliphatic group. Exemplary divalent groups include ethylene, 1,4-butylene, 1,2-propylene, 2,2-dimethyl-1,3-propylene and 1,5-pentylene. Exemplary tri- and tetra-valent groups are shown below.

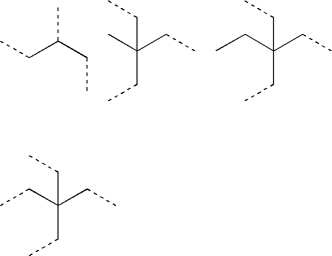

Herein the broken line denotes a valence bond. These formulae are partial structures derived from glycerol, trimethylol ethane, trimethylol propane, and pentaerythritol, respectively. Of these, 1,4-butylene and 2,2-dimethyl-1,3-propylene are preferably used.

Rf is trifluoromethyl or pentafluoroethyl, and preferably trifluoromethyl. The letter m′ is an integer of 0 to 3, n′ is an integer of 1 to 4, and the sum of m′ and n′, which represents the valence of R, is an integer of 2 to 4. A is equal to 1, B is an integer of 2 to 25, and C is an integer of 0 to 10. Preferably, B is an integer of 4 to 20, and C is 0 or 1. Note that the above structural formula does not prescribe the arrangement of respective constituent units while they may be arranged either in blocks or randomly. For the preparation of surfactants in the form of partially fluorinated oxetane ring-opened polymers, reference should be made to U.S. Pat. No. 5,650,483, for example.

Of the foregoing surfactants, FC-4430, Surflon S-381, KH-20, KH-30, and oxetane ring-opened polymers of formula (surf-1) are preferred. These surfactants may be used alone or in admixture.

In the resist composition, the surfactant is preferably compounded in an amount of up to 2 parts, and especially up to 1 part by weight, per 100 parts by weight of the base resin. The amount of the surfactant, if added, is preferably at least 0.01 phr.

Optionally, there may be added to the resist composition of the invention a polymer which will locally segregate at the top of a coating and functions to adjust a hydrophilic/hydrophobic balance at the surface, to enhance water repellency, or to prevent low-molecular-weight components from flowing into or out of the coating when the coating comes in contact with water or similar liquids. The functional polymer may be added in customary amounts as long as it does not compromise the objects of the invention.

Preferred examples of the functional polymer which will segregate at the coating top include polymers and copolymers comprising fluorinated units of one or more types, and copolymers comprising fluorinated units and other units. Illustrative examples of suitable fluorinated units and other units are shown below, but not limited thereto.

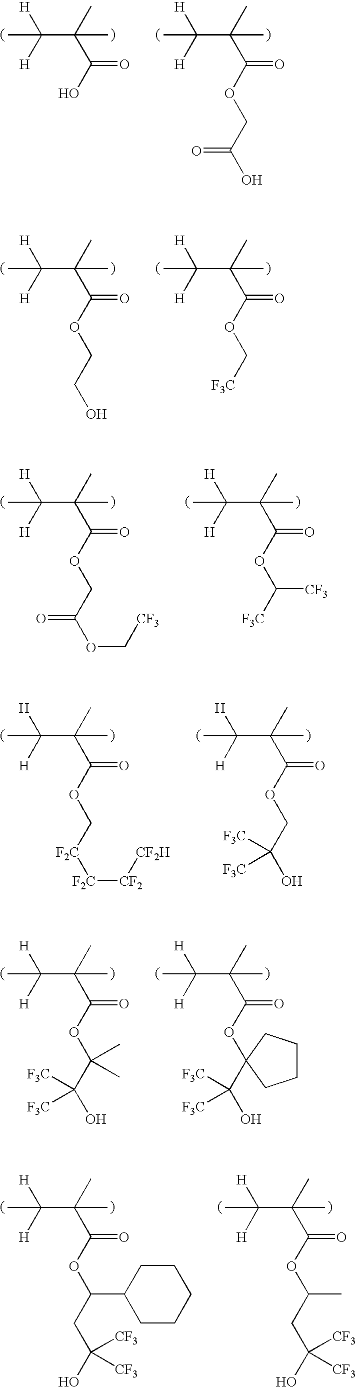

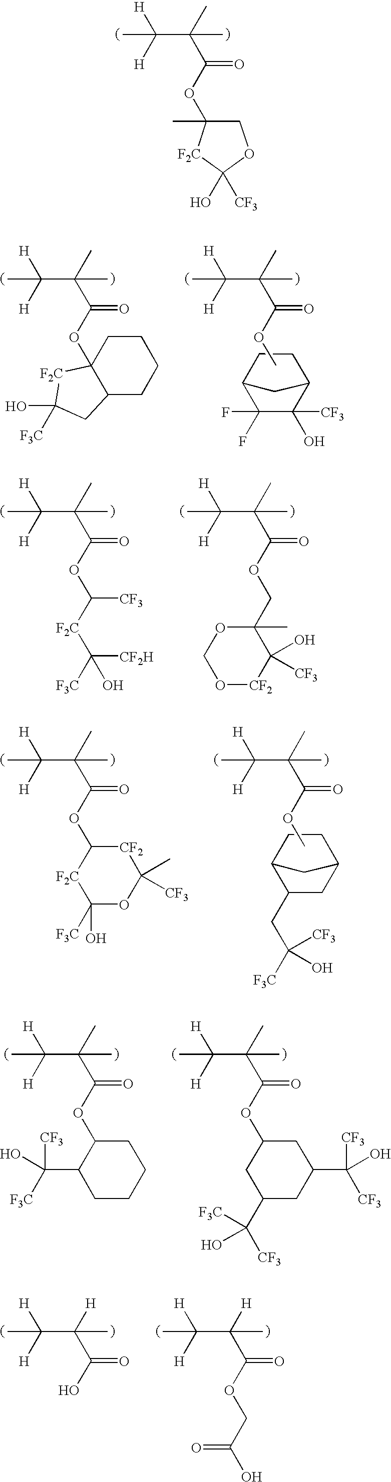

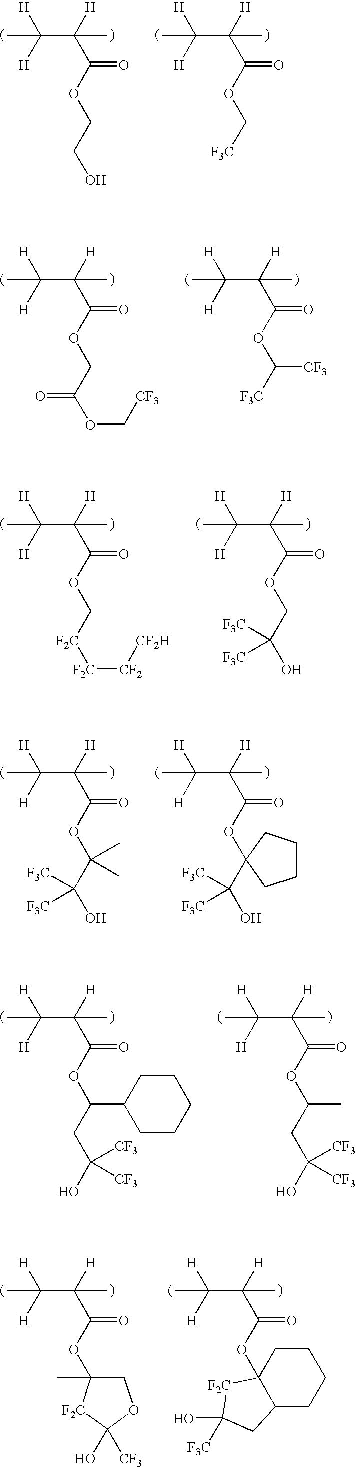

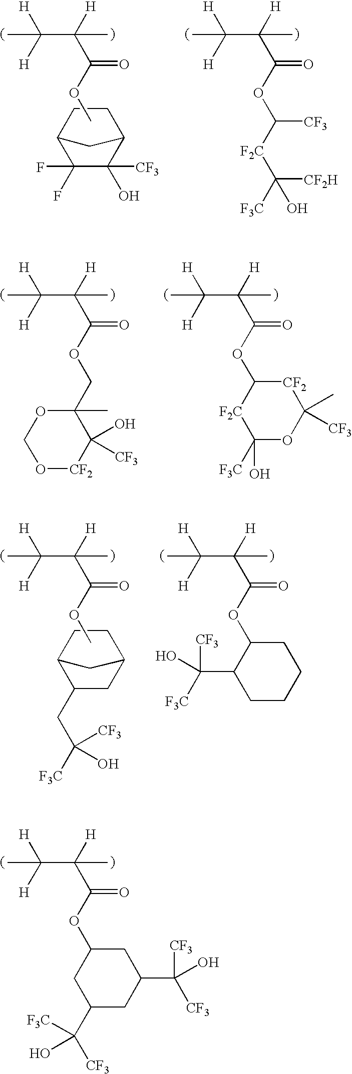

The functional polymer which will segregate at the coating top should preferably have a weight average molecular weight of 1,000 to 50,000, more preferably 2,000 to 20,000, as measured by GPC versus polystyrene standards. Outside the range, the polymer may have insufficient surface-modifying effect or cause development defects. The preferred amount of the functional polymer added is 0 to 10 parts, more preferably 0 to 5 parts by weight per 100 parts by weight of the base polymer, and when added, at least 1 part by weight.

To the resist composition of the invention, other components such as dissolution regulators, carboxylic acid compounds and acetylene alcohol derivatives may be added if necessary. Optional components may be added in conventional amounts so long as this does not compromise the objects of the invention.

The dissolution regulator which can be added to the resist composition is a compound having on the molecule at least two phenolic hydroxyl groups, in which an average of from 0 to 100 mol % of all the hydrogen atoms on the phenolic hydroxyl groups are replaced by acid labile groups or a compound having on the molecule at least one carboxyl group, in which an average of 50 to 100 mol % of all the hydrogen atoms on the carboxyl groups are replaced by acid labile groups, both the compounds having a weight average molecular weight within a range of 100 to 1,000, and preferably 150 to 800.

The degree of substitution of the hydrogen atoms on the phenolic hydroxyl groups with acid labile groups is on average at least 0 mol %, and preferably at least 30 mol %, of all the phenolic hydroxyl groups. The upper limit is 100 mol %, and preferably 80 mol %. The degree of substitution of the hydrogen atoms on the carboxyl groups with acid labile groups is on average at least 50 mol %, and preferably at least 70 mol %, of all the carboxyl groups, with the upper limit being 100 mol %.

Preferable examples of such compounds having two or more phenolic hydroxyl groups or compounds having a carboxyl group include those of formulas (D1) to (D14) below.

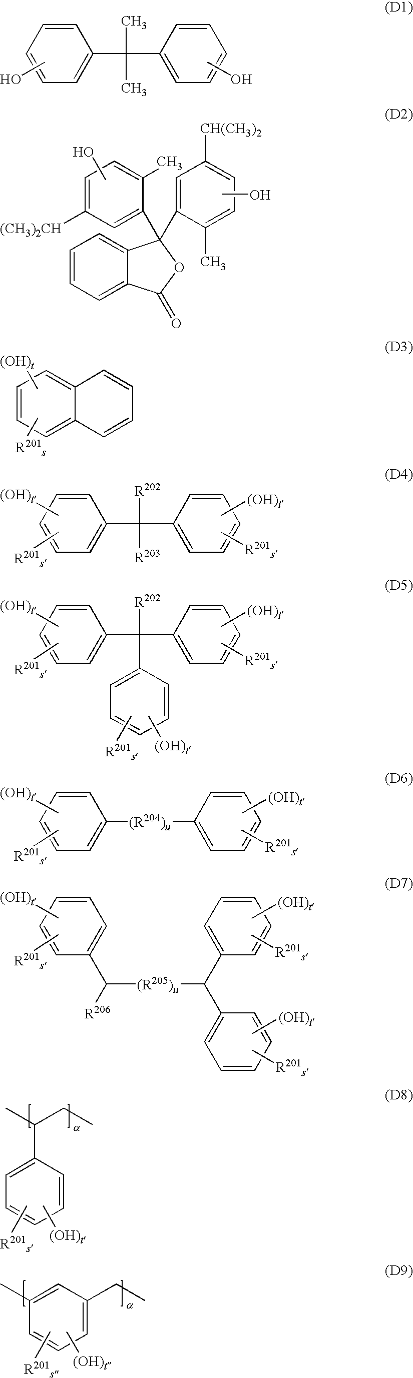

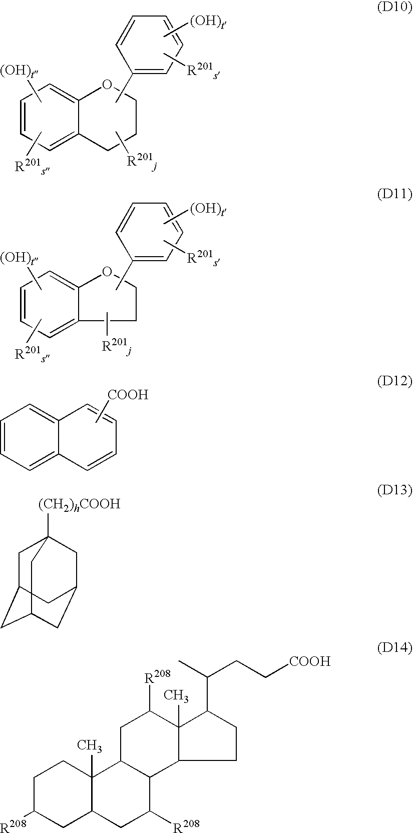

In these formulas, R²⁰¹ and R²⁰² are each hydrogen or a straight or branched C₁-C₈ alkyl or alkenyl group, for example, hydrogen, methyl, ethyl, butyl, propyl, ethynyl and cyclohexyl.

R²⁰³ is hydrogen, a straight or branched C₁-C₈ alkyl or alkenyl group, or —(R²⁰⁷)ₕ—COOH (wherein R²⁰⁷ is a straight or branched C₁-C₁₀ alkylene), for example, those exemplified for R²⁰¹ and R²⁰², —COOH or —CH₂COOH.

R²⁰⁴ is —(CH₂)ᵢ— wherein i=2 to 10, C₆-C10 arylene, carbonyl, sulfonyl, an oxygen atom, or a sulfur atom, for example, ethylene, phenylene, carbonyl, sulfonyl, oxygen atom or sulfur atom.

R²⁰⁵ is a C₁-C₁₀ alkylene, a C₆-C₁₀ arylene, carbonyl, sulfonyl, an oxygen atom, or a sulfur atom, for example, methylene and those exemplified for R²⁰⁴.

R²⁰⁶ is hydrogen, a straight or branched C₁-C₆ alkyl or alkenyl, or a phenyl or naphthyl group in which at least one hydrogen atom is substituted by a hydroxyl group, for example, hydrogen, methyl, ethyl, butyl, propyl, ethynyl, cyclohexyl, hydroxyl-substituted phenyl, and hydroxyl-substituted naphthyl.

R²⁰⁸ is hydrogen or hydroxyl.

The letter j is an integer from 0 to 5; u and h are each 0 or 1; s, t, s′, t′, s″, and t″ are each numbers which satisfy s+t =8, s′+t′=5, and s″+t″=4, and are such that each phenyl structure has at least one hydroxyl group; and α is a number such that the compounds of formula (D8) or (D9) have a weight average molecular weight of from 100 to 1,000.

Exemplary acid labile groups on the dissolution regulator include a variety of such groups, typically groups of the general formulae (L1) to (L4), tertiary C₄-C20 alkyl groups, trialkylsilyl groups in which each of the alkyls has 1 to 6 carbon atoms, and C₄-C₂₀ oxoalkyl groups. Examples of the respective groups are as previously described.

The dissolution regulator may be formulated in an amount of 0 to 50 parts, preferably 0 to 40 parts, and more preferably 0 to 30 parts by weight, per 100 parts by weight of the base polymer, and may be used singly or as a mixture of two or more thereof. The use of more than 50 parts of the dissolution regulator may lead to slimming of the patterned film, and thus a decline in resolution.

The dissolution regulator can be synthesized by introducing acid labile groups into a compound having phenolic hydroxyl or carboxyl groups in accordance with an organic chemical formulation.

In the resist composition, a carboxylic acid compound may be blended. The carboxylic acid compound used herein may be one or more compounds selected from Groups I and II below, but is not limited thereto. Including this compound improves the PED stability of the resist and ameliorates edge roughness on nitride film substrates.

### Group I:

Compounds of general formulas (A1) to (A10) below in which some or all of the hydrogen atoms on the phenolic hydroxyl groups are replaced by —R⁴⁰¹—COOH (wherein R⁴⁰¹ is a straight or branched C₁-C₁₀ alkylene group), and in which the molar ratio C/(C+D) of phenolic hydroxyl groups (C) to ≡C—COOH groups (D) in the molecule is from 0.1 to 1.0.

### Group II:

Compounds of general formulas (A11) to (A15) below.

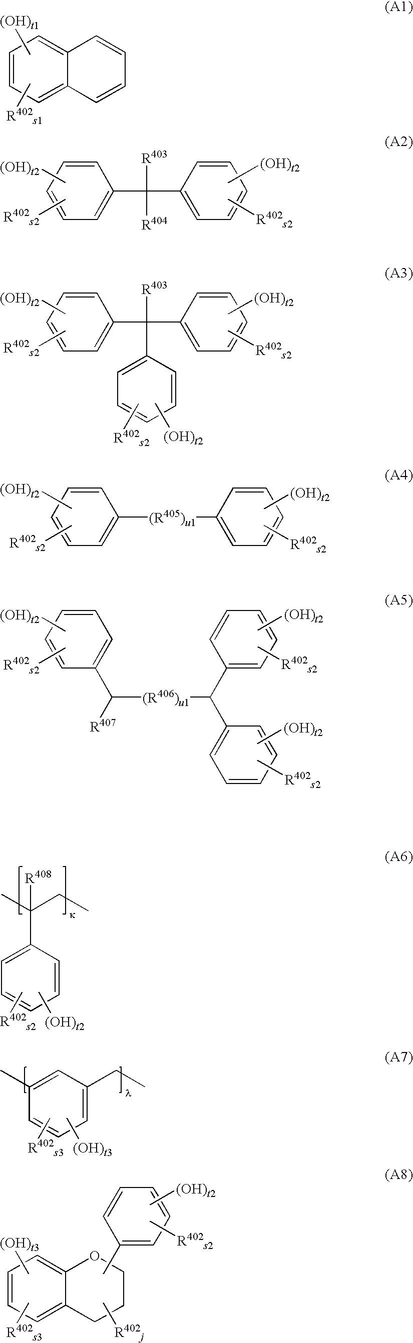

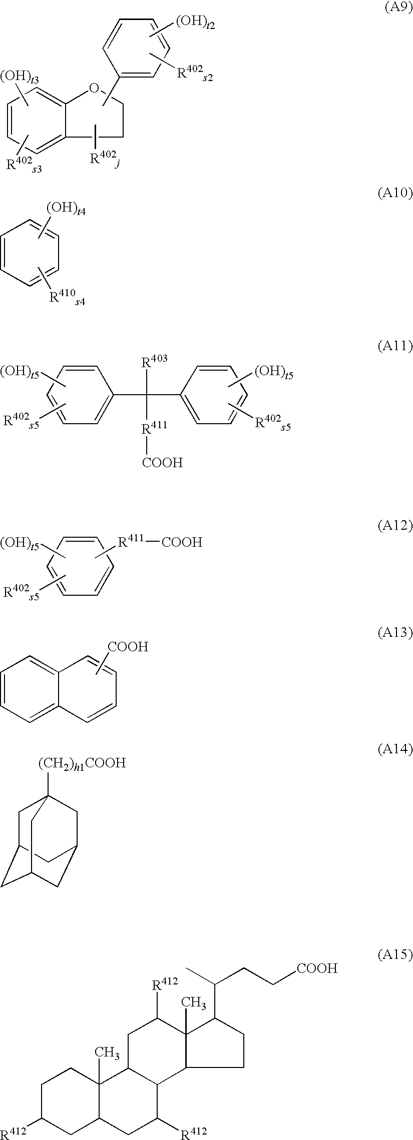

In these formulas, R⁴⁰² and R⁴⁰³ are each hydrogen or a straight or branched C₁-C₈ alkyl or alkenyl. R⁴⁰⁴ is hydrogen, a straight or branched C₁-C₈ alkyl or alkenyl, or a —(R⁴⁰⁹)ₕ₁—COOR′ group wherein R⁴⁰⁷ is hydrogen or —R⁴⁰⁹—COOH.

R⁴⁰⁵ is —(CH₂)ᵢ— (wherein i is 2 to 10), a C₆-C₁₀ arylene, carbonyl, sulfonyl, an oxygen atom, or a sulfur atom. R⁴⁰⁹ is a C₁-C₁₀ alkylene, a C₆-C₁₀ arylene, carbonyl, sulfonyl, an oxygen atom, or a sulfur atom. R⁴⁰⁷ is hydrogen, a straight or branched C₁-C₈ alkyl or alkenyl, or a hydroxyl-substituted phenyl or naphthyl. R⁴⁰⁸ is hydrogen or methyl. R⁴⁰⁹ is a straight or branched C₁-C₁₀ alkylene. R⁴¹⁰ is hydrogen, a straight or branched C₁-C₈ alkyl or alkenyl, or a —R⁴¹¹—COOH group wherein R⁴¹¹ is a straight or branched C₁-C₁₀ alkylene. R⁴¹² is hydrogen or hydroxyl.

The letter j is a number from 0 to 3; s1, t1, s2, t2, s3, t3, s4, and t4 are each numbers which satisfy s1+t1=8, s2+t2=5, s3+t3=4, and s4+t4=6, and are such that each phenyl structure has at least one hydroxyl group; s5 and t5 are numbers which satisfy s5≧0, t5≧0, and s5+t5=5; u1 is a number from 1 to 4; h1 is a number from 0 to 4; κ is a number such that the compound of formula (A6) may have a weight average molecular weight of 1,000 to 5,000; and λ is a number such that the compound of formula (A7) may have a weight average molecular weight of 1,000 to 10,000.

Illustrative, non-limiting examples of the compound having a carboxyl group include compounds of the general formulas (AI-1) to (AI-14) and (AII-1) to (AII-10) below.

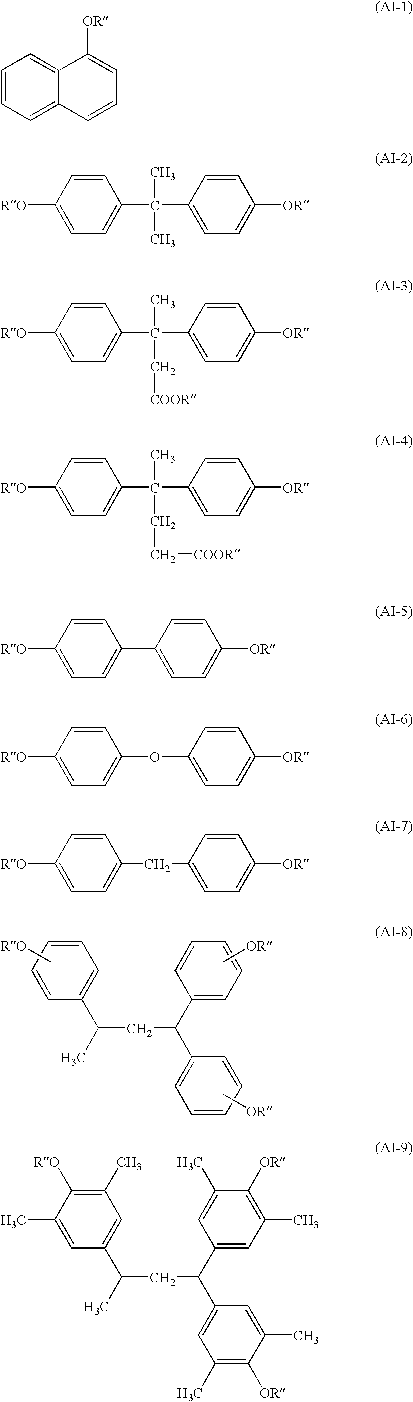

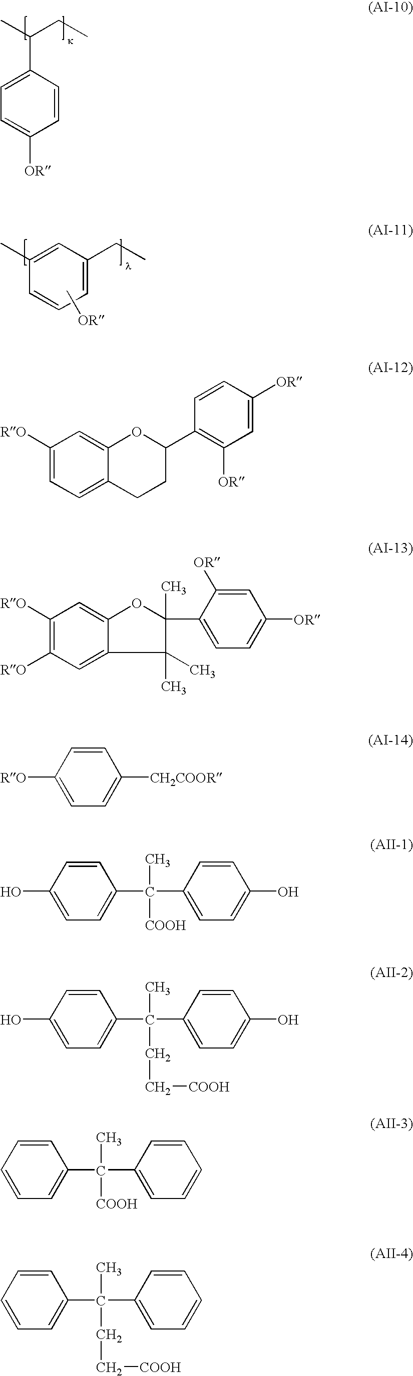

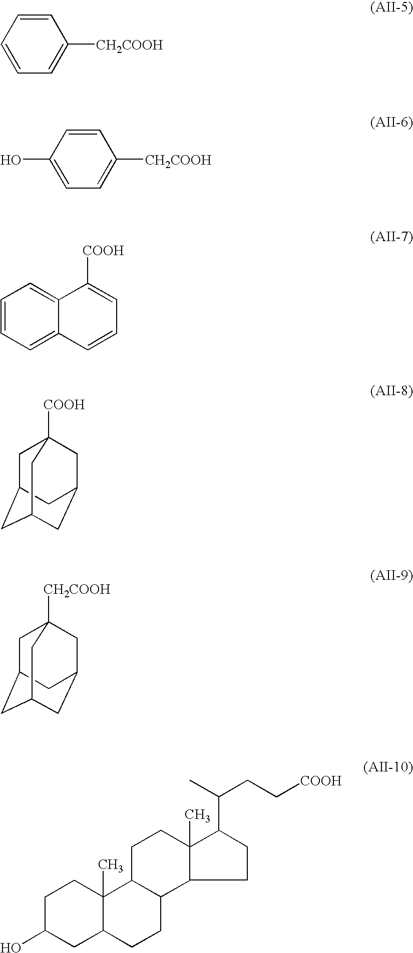

In the above formulas, R″ is hydrogen or a —CH₂COOH group such that the —CH₂COOH group accounts for 10 to 100 mol % of R″ in each compound, κ and λ are as defined above.

The compound having a ≡C—COOH group may be used singly or as combinations of two or more thereof. The compound having a ≡C—COOH group is added in an amount ranging from 0 to 5 parts, preferably 0.1 to 5 parts, more preferably 0.1 to 3 parts, further preferably 0.1 to 2 parts by weight, per 100 parts by weight of the base polymer. More than 5 parts of the compound can reduce the resolution of the resist composition.

Preferred examples of the acetylene alcohol derivative which can be added to the resist composition include those having the general formula (S1) or (S2) below.

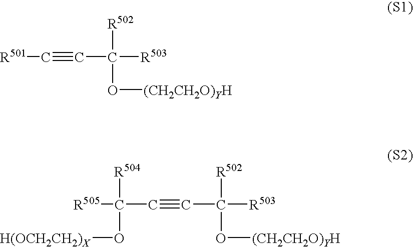

In the formulas, R⁵⁰¹, R⁵⁰², R⁵⁰³, R⁵⁰⁴, and R⁵⁰⁵ are each hydrogen or a straight, branched or cyclic C₁-C₈ alkyl; and X and Y are each 0 or a positive number, satisfying 0≦X≦30, 0≦Y≦30, and 0≦X+Y≦40.

Preferable examples of the acetylene alcohol derivative include Surfynol 61, Surfynol 82, Surfynol 104, Surfynol 104E, Surfynol 104H, Surfynol 104A, Surfynol TG, Surfynol PC, Surfynol 440, Surfynol 465, and Surfynol 485 from Air Products and Chemicals Inc., and Surfynol E1004 from Nisshin Chemical Industries Ltd.

The acetylene alcohol derivative is preferably added in an amount of 0 to 2 parts, more preferably 0.01 to 2 parts, and even more preferably 0.02 to 1 part by weight per 100 parts by weight of the base polymer in the resist composition. More than 2 parts by weight may result in a resist having a low resolution.

### Process

Pattern formation using the resist composition of the invention may be performed by well-known lithography processes. The process generally involves coating, heat treatment (or prebaking), exposure, heat treatment (post-exposure baking, PEB), and development. If necessary, any other steps may be added.

For pattern formation, the resist composition is first applied onto a substrate (on which an integrated circuit is to be formed, e.g., Si, SiO₂, SiN, SiON, TiN, WSi, BPSG, SOG, organic antireflective coating, Cr, CrO, CrON, MoSi, etc.) by a suitable coating technique such as spin coating, roll coating, flow coating, dip coating, spray coating or doctor coating. The coating is prebaked on a hot plate at a temperature of 60 to 150° C. for about 1 to 10 minutes, preferably 80 to 140° C. for 1 to 5 minutes. The resulting resist film is generally 0.01 to 2.0 μm thick.

A relationship of a reduced thickness of resist film to an etch selectivity ratio between resist film and processable substrate imposes severer limits on the process. Under consideration is the tri-layer process in which a resist layer, a silicon-containing intermediate layer, an undercoat layer having a high carbon density and high etch resistance, and a processable substrate are laminated in sequence from top to bottom. On etching with oxygen gas, hydrogen gas, ammonia gas or the like, a high etch selectivity ratio is available between the silicon-containing intermediate layer and the undercoat layer, which allows for thickness reduction of the silicon-containing intermediate layer. A relatively high etch selectivity ratio is also available between the monolayer resist and the silicon-containing intermediate layer, which allows for thickness reduction of the monolayer resist. The method for forming the undercoat layer in this case includes a coating and baking method and a CVD method. In the case of coating, novolac resins and resins obtained by polymerization of fused ring-containing olefins are used. In the CVD film formation, gases such as butane, ethane, propane, ethylene and acetylene are used. For the silicon-containing intermediate layer, either a coating method or a CVD method may be employed. The coating method uses silsesquioxane, polyhedral oligomeric silsesquioxane (POSS) and the like while the CVD method uses silane gases as the reactant. The silicon-containing intermediate layer may have an antireflection function with a light absorbing ability and have photo-absorptive groups like phenyl groups, or it may be a SiON film. An organic film may be formed between the silicon-containing intermediate layer and the photoresist, and the organic film in this case may be an organic antireflective coating. After the photoresist film is formed, pure water rinsing (or post-soaking) may be carried out for extracting the acid generator and the like from the film surface or washing away particles, or a protective film may be coated.

With a mask having a desired pattern placed above the resist film, the resist film is then exposed to actinic radiation such as UV, deep-UV, electron beams, x-rays, excimer laser light, γ-rays and synchrotron radiation. The exposure dose is preferably about 1 to 200 mJ/cm², more preferably about 10 to 100 mJ/cm². The film is then baked on a hot plate at 60 to 150° C. for 1 to 5 minutes, preferably 80 to 120° C. for 1 to 3 minutes (post-exposure baking=PEB). Thereafter the resist film is developed with a developer in the form of an aqueous base solution, for example, 0.1 to 5 wt %, preferably 2 to 3 wt % aqueous solution of tetramethylammonium hydroxide (TMAH) for 0.1 to 3 minutes, preferably 0.5 to 2 minutes by conventional techniques such as dip, puddle or spray techniques. In this way, a desired resist pattern is formed on the substrate. It is appreciated that the resist composition of the invention is suited for micropatterning using such high-energy radiation as deep UV with a wavelength of 254 to 193 nm, vacuum UV with a wavelength of 157 nm, electron beams, soft x-rays, x-rays, excimer laser light, γ-rays and synchrotron radiation, and best suited for micropatterning using high-energy radiation in the wavelength range of 180 to 200 nm.

Immersion lithography can be applied to the resist composition of the invention. The ArF immersion lithography uses a liquid having a refractive index of at least 1 and transparent to exposure radiation, such as pure water or alkanes as the immersion solvent. The immersion lithography involves prebaking a resist film and exposing the resist film to light through a projection lens, with pure water or similar liquid interposed between the resist film and the projection lens. Since this allows projection lenses to be designed to a numerical aperture (NA) of 1.0 or higher, formation of finer patterns is possible. The immersion lithography is important for the ArF lithography to survive to the 45-nm node, with a further development thereof being accelerated. In the case of immersion lithography, pure water rinsing (or post-soaking) may be carried out after exposure for removing water droplets left on the resist film, or a protective coating may be applied onto the resist film after pre-baking for preventing any dissolution from the resist and improving water slip on the film surface. The resist protective coating used in the immersion lithography is preferably formed from a solution of a polymer having 1,1,1,3,3,3-hexafluoro-2-propanol residue which is insoluble in water and soluble in an alkaline developer liquid, in a solvent selected from alcohols of at least 4 carbon atoms, ethers of 8 to 12 carbon atoms, and mixtures thereof.

The technique enabling the ArF lithography to survive to the 32-nm node is a double patterning process. The double patterning process includes a trench process of processing an underlay to a 1:3 trench pattern by a first step of exposure and etching, shifting the position, and forming a 1:3 trench pattern by a second step of exposure, for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

### Example

Examples of the invention are given below by way of illustration and not by way of limitation. Mw is a weight average molecular weight as measured by gel permeation chromatography (GPC) versus polystyrene standards.

### Synthesis Example 1

### Synthesis of 4-hydroxy-adamantan-1-yl methacrylate (Monomer 1)

### Synthesis Example 1-1

### Synthesis of 4-oxo-adamantan-1-yl methacrylate (Monomer Intermediate 1)

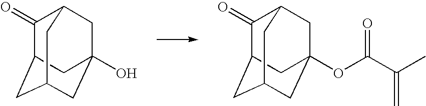

Under ice cooling, 75 g of methacryloyl chloride was added dropwise to a mixture of 100 g of 5-hydroxy-adamantan-2-one, 79 g of triethylamine, 0.1 g of 2,2′-methylenebis(6-t-butyl-p-cresol), and 400 g of methylene chloride. The mixture was stirred at room temperature for 15 hours, after which 500 g of 10% hydrochloric acid was added to quench the reaction. The organic layer was separated, washed with water, and concentrated. To remove the residual methacrylic anhydride, the concentrate was dissolved in 500 g of toluene. Then 300 g of a saturated sodium hydrogencarbonate aqueous solution and 0.5 g of 4-dimethylaminopyridine were added to the solution, which was stirred for 2 hours. The organic layer was separated, washed with water, and concentrated. To the concentrate, 450 g of toluene, 7.5 g of activated carbon, and 7.5 g of neutral alumina were added for adsorption treatment. After Celite® filtration, the reaction solution was concentrated, obtaining 120 g of the target compound as oily matter (yield 70%). 4-oxo-adamantan-1-yl methacrylate (Monomer Intermediate 1) GC-MS (EI): (m/z)⁺=41, 55, 69, 92, 104, 120, 148, 234 (M⁺)

### Synthesis Example 1-2

### Synthesis of 4-hydroxy-adamantan-1-yl methacrylate (Monomer 1)

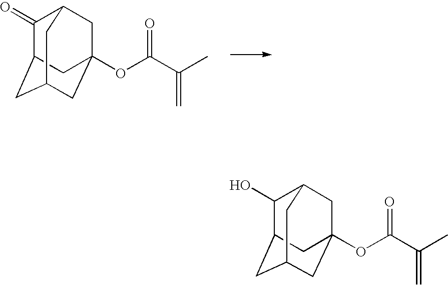

Under ice cooling, a mixture of 120 g of 4-oxo-adamantan-1-yl methacrylate and 240 g of tetrahydrofuran was added dropwise to a mixture of 16 g of sodium borohydride, 480 g of tetrahydrofuran and 0.1 g of 2,2′-methylenebis(6-t-butyl-p-cresol). After one hour of stirring, 550 g of 5% hydrochloric acid was added to quench the reaction. Ethyl acetate was added to the reaction solution, whereupon the organic layer was separated, washed with water, and concentrated. Hexane was added to the concentrate for recrystallization, obtaining 64 g of the target compound as white solids (yield 63%).

4-hydroxy-adamantan-1-yl methacrylate (Monomer 1) (mixture of ca. 70:30 isomers)

IR (KBr disk):
v=3155, 2939, 2912, 2865, 2852, 1704, 1454, 1376, 1361, 1328, 1301, 1292, 1178, 1105, 1091, 1064, 1045, 1037, 937, 811 cm⁻¹

¹H—NMR (600 MHz in DMSO-d₆, major isomer):
δ=1.30 (2Hx0.7H, app d, J=11.7 Hz), 1.53 (2Hx0.3H, app d, J=12.4 Hz), 1.64 (2Hx0.3H, app d, J=11.3 Hz), 1.79 (2Hx0.3H, s), 1.80 (3H, t, J=1.1, 1.5 Hz), 1.92 (2Hx0.7H, s), 1.98-2.08 (8H, m), 2.32 (2Hx0.3H, app d, J=10.7 Hz), 3.54 (1x0.3H, q, J=3.0 Hz), 3.71 (1x0.7H, q, J=3.5 Hz), 4.67 (1x0.7H, d, J=3.1 Hz), 4.70 (1x0.3H, d, J=3.0 Hz), 5.55-5.57 (1H, m), 5.89-5.91 (1H, m) ppm

¹³C-NMR (150 MHz in DMSO-d₆):
δ=17.96, 17.99, 29.07, 29.37, 29.55, 34.37, 34.66, 35.81, 36.82, 40.54, 40.83, 70.41, 71.04, 79.08, 79.45, 124.52, 124.69, 137.26, 137.36, 165.36, 165.49 ppm

GC-MS (EI): (m/z)⁻=41, 58, 79, 91, 106, 121, 133, 150, 236 (M⁺⁾

### Preparation of Resist Material

### Examples

Positive resist compositions were prepared by dissolving a polymer, acid generator, and basic compound in a solvent in accordance with the formulation shown in Table 1 and filtering through a Teflon® filter with a pore size of 0.2 μm. In all runs, the solvent contained 0.005 wt % of surfactant KH-20 (Asahi Glass Co., Ltd.).

TABLE 1

| | | Acid | | | |
|---|---|---|---|---|---|
| Resist | Resin | generator | Base | Solvent 1 | Solvent 2 |
| R-01 | P-01 (80) | PAG-1 (10.1) | Base-1 (1.41) | PGMEA (1,120) | CyHO (480) |
| R-02 | P-02 (80) | PAG-1 (10.1) | Base-1 (1.41) | PGMEA (1,120) | CyHO (480) |
| R-03 | P-03 (80) | PAG-1 (10.1) | Base-1 (1.41) | PGMEA (1,120) | CyHO (480) |
| R-04 | P-04 (80) | PAG-1 (10.1) | Base-1 (1.41) | PGMEA (1,120) | CyHO (480) |
| R-05 | P-05 (80) | PAG-1 (10.1) | Base-1 (1.41) | PGMEA (1,120) | CyHO (480) |
| R-06 | P-06 (80) | PAG-1 (10.1) | Base-1 (1.41) | PGMEA (1,120) | CyHO (480) |
| R-07 | P-07 (80) | PAG-1 (10.1) | Base-1 (1.41) | PGMEA (1,120) | CyHO (480) |
| R-08 | P-08 (80) | PAG-1 (10.1) | Base-1 (1.41) | PGMEA (1,120) | CyHO (480) |
| R-09 | P-09 (80) | PAG-1 (10.1) | Base-1 (1.41) | PGMEA (1,120) | CyHO (480) |
| R-10 | P-10 (80) | PAG-1 (10.1) | Base-1 (1.41) | PGMEA (1,120) | CyHO (480) |
| R-11 | P-11 (80) | PAG-1 (10.1) | Base-1 (1.41) | PGMEA (1,120) | CyHO (480) |
| R-12 | P-12 (80) | PAG-1 (10.1) | Base-1 (1.41) | PGMEA (1,120) | CyHO (480) |
| R-13 | P-13 (80) | PAG-1 (10.1) | Base-1 (1.41) | PGMEA (1,120) | CyHO (480) |
| R-14 | P-14 (80) | PAG-1 (10.1) | Base-1 (1.41) | PGMEA (1,120) | CyHO (480) |
| R-15 | P-15 (80) | PAG-1 (10.1) | Base-1 (1.41) | PGMEA (1,120) | CyHO (480) |
| R-16 | P-16 (80) | PAG-1 (10.1) | Base-1 (1.41) | PGMEA (1,120) | CyHO (480) |
| R-17 | P-17 (80) | PAG-1 (10.1) | Base-1 (1.41) | PGMEA (1,120) | CyHO (480) |
| R-18 | P-18 (80) | PAG-1 (10.1) | Base-1 (1.41) | PGMEA (1,120) | CyHO (480) |
| R-19 | P-19 (80) | PAG-1 (10.1) | Base-1 (1.41) | PGMEA (1,120) | CyHO (480) |
| R-20 | P-20 (80) | PAG-1 (10.1) | Base-1 (1.41) | PGMEA (1,120) | CyHO (480) |
| R-21 | P-01 (80) | PAG-2 (11.0) | Base-1 (1.41) | PGMEA (1,120) | CyHO (480) |
| R-22 | P-01 (80) | PAG-3 (10.0) | Base-1 (1.41) | PGMEA (1,120) | CyHO (480) |
| R-23 | P-01 (80) | PAG-4 (10.2) | Base-1 (1.41) | PGMEA (1,120) | CyHO (480) |
| R-24 | P-01 (80) | PAG-5 (12.6) | Base-1 (1.41) | PGMEA (1,120) | CyHO (480) |
| R-25 | P-01 (80) | PAG-6 (10.4) | Base-1 (1.41) | PGMEA (1,120) | CyHO (480) |
| R-26 | P-20 (80) | PAG-2 (11.0) | Base-1 (1.41) | PGMEA (1,120) | CyHO (480) |
| R-27 | P-20 (80) | PAG-3 (10.0) | Base-1 (1.41) | PGMEA (1,120) | CyHO (480) |
| R-28 | P-20 (80) | PAG-4 (10.2) | Base-1 (1.41) | PGMEA (1,120) | CyHO (480) |
| R-29 | P-20 (80) | PAG-5 (12.6) | Base-1 (1.41) | PGMEA (1,120) | CyHO (480) |
| R-30 | P-20 (80) | PAG-6 (10.4) | Base-1 (1.41) | PGMEA (1,120) | CyHO (480) |

The values in parentheses are in parts by weight.

### Comparative Examples

Resist compositions for comparison were prepared by the same procedure as in Examples aside from using the formulation shown in Table 2.

TABLE 2

| | | Acid | | | |
|---|---|---|---|---|---|
| Resist | Resin | generator | Base | Solvent 1 | Solvent 2 |
| R-31 | P-21 (80) | PAG-1 (10.1) | Base-1 (1.41) | PGMEA (1,120) | CyHO (480) |
| R-32 | P-22 (80) | PAG-1 (10.1) | Base-1 (1.41) | PGMEA (1,120) | CyHO (480) |
| R-33 | P-23 (80) | PAG-1 (10.1) | Base-1 (1.41) | PGMEA (1,120) | CyHO (480) |
| R-34 | P-24 (80) | PAG-1 (10.1) | Base-1 (1.41) | PGMEA (1,120) | CyHO (480) |

The values in parentheses are in parts by weight.

In Tables 1 and 2, the base and solvent are designated by abbreviations, which have the following meaning.
Base-1: tri(2-methoxymethoxyethyl)amine
PGMEA: propylene glycol monomethyl ether acetate
CyHO: cyclohexanone

The resins designated by abbreviations are polymers constructed as in Tables 3 to 6.

TABLE 3

| Resin | Unit 1 | Unit 2 | Unit 3 | Unit 4 | Unit 5 | Mw |
|---|---|---|---|---|---|---|
| P-01 | A-1M (0.30) | B-1M (0.25) | C-4M (0.45) | | | 6,900 |
| P-02 | A-2M (0.30) | B-1M (0.25) | C-4M (0.45) | | | 6,000 |
| P-03 | A-3M (0.30) | B-1M (0.25) | C-4M (0.45) | | | 7,000 |
| P-04 | A-4M (0.30) | B-1M (0.25) | C-4M (0.45) | | | 6,500 |
| P-05 | A-5M (0.30) | B-1M (0.25) | C-4M (0.45) | | | 6,700 |
| P-06 | A-6M (0.30) | B-1M (0.25) | C-4M (0.45) | | | 7,100 |
| P-07 | A-1M (0.30) | B-1M (0.25) | C-4A (0.45) | | | 6,400 |
| P-08 | A-1M (0.30) | B-2M (0.25) | C-4M (0.45) | | | 6,900 |
| P-09 | A-1M (0.30) | B-1M (0.25) | C-1M (0.45) | | | 6,600 |
| P-10 | A-1M (0.30) | B-1M (0.25) | C-2M (0.45) | | | 6,500 |
| P-11 | A-1M (0.30) | B-1M (0.25) | C-3M (0.45) | | | 6,700 |
| P-12 | A-1M (0.30) | B-1M (0.25) | C-2M (0.35) | C-5M (0.10) | | 6,700 |
| P-13 | A-1M (0.30) | B-1M (0.25) | C-2M (0.35) | C-6M (0.10) | | 6,800 |
| P-14 | A-1M (0.30) | B-1M (0.25) | C-2M (0.35) | C-7M (0.10) | | 6,600 |
| P-15 | A-1M (0.25) | B-1M (0.25) | C-4M (0.40) | C-8M (0.10) | | 6,500 |
| P-16 | A-1M (0.25) | B-1M (0.25) | C-4M (0.40) | C-9M (0.10) | | 6,400 |
| P-17 | A-1M (0.25) | B-1M (0.25) | C-4M (0.40) | C-10M (0.10) | | 6,400 |
| P-18 | A-1M (0.25) | B-1M (0.25) | C-4M (0.40) | C-11M (0.10) | | 6,200 |
| P-19 | A-1M (0.20) | A-4M (0.10) | B-1M (0.25) | C-2M (0.35) | C-6M (0.10) | 6,600 |
| P-20 | A-1M (0.20) | A-4M (0.10) | B-1M (0.25) | C-2M (0.35) | C-7M (0.10) | 6,500 |
| P-21 | A-1M (0.30) | B-3M (0.25) | C-4M (0.45) | | | 6,900 |
| P-22 | A-1M (0.30) | B-4M (0.25) | C-4M (0.45) | | | 6,700 |
| P-23 | A-1M (0.25) | B-3M (0.25) | C-4M (0.40) | C-9M (0.10) | | 6,500 |
| P-24 | A-1M (0.20) | A-4M (0.10) | B-3M (0.25) | C-2M (0.35) | C-7M (0.10) | 6,400 |

The value in parentheses is an incorporation ratio of a particular unit expressed in molar ratio.

TABLE 4

| | |
|---|---|
| 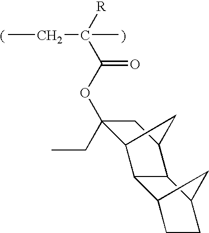 | 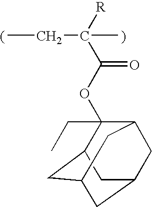 |
| A-1M (R = CH₃) | A-2M (R = CH₃) |
| A-1A (R = H) | A-2A (R = H) |

| | |
|---|---|
| 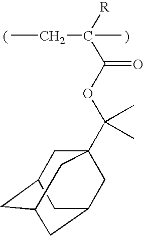 | 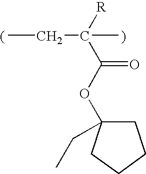 |
| A-3M (R = CH₃) | A-4M (R = CH₃) |
| A-3A (R = H) | A-4A (R = H) |

| | |
|---|---|
| 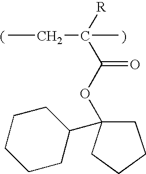 | 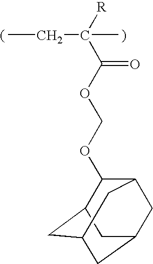 |
| A-5M (R = CH₃) | A-6M (R = CH₃) |
| A-5A (R = H) | A-6A (R = H) |

TABLE 5

| | |
|---|---|
| 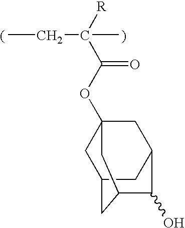 | 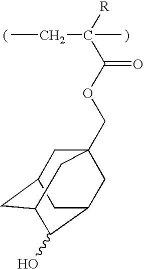 |
| B-1M (R = CH₃) | B-2M (R = CH₃) |
| B-1A (R = H) | B-2A (R = H) |

| | |
|---|---|
| 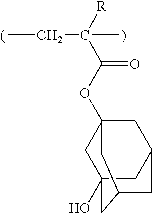 | 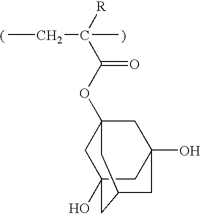 |
| B-3M (R = CH₃) | B-4M (R = CH₃) |
| B-3A (R = H) | B-4A (R = H) |

TABLE 6

| 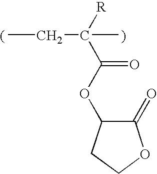 | | 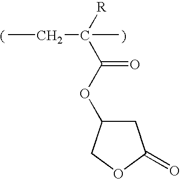 |
|---|---|---|
| C-1M (R = CH₃) | | C-2M (R = CH₃) |
| C-1A (R = H) | | C-2A (R = H) |
| | 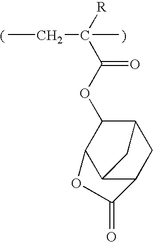 | 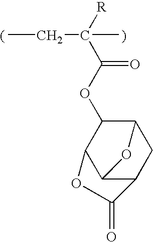 |
| | C-3M (R = CH₃) | C-4M (R = CH₃) |
| | C-3A (R = H) | C-4A (R = H) |
| | 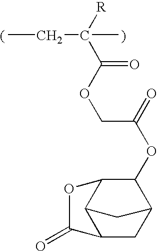 | 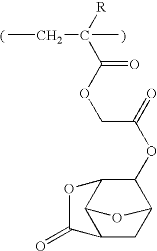 |
| | C-5M (R = CH₃) | C-6M (R = CH₃) |
| | C-5A (R = H) | C-6A (R = H) |

| | |
|---|---|
| 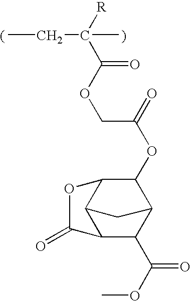 | 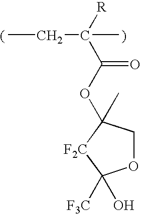 |
| C-7M (R = CH₃) | C-8M (R = CH₃) |
| C-7A (R = H) | C-8A (R = H) |

| | |
|---|---|
| 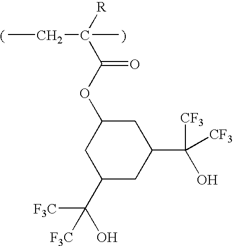 | 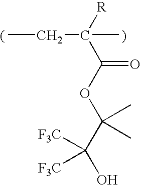 |
| C-9M (R = CH₃) | C-10M (R = CH₃) |
| C-9A (R = H) | C-10A (R = H) |

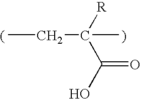

C-11M (R = CH₃)
C-11A (R = H)

The acid generators designated by abbreviations in Tables 1 and 2 are sulfonium salts shown in Table 7.

TABLE 7

PAG-1

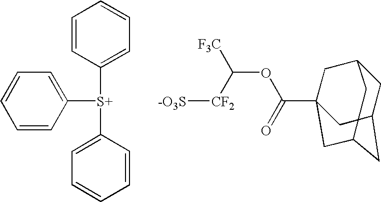

PAG-2

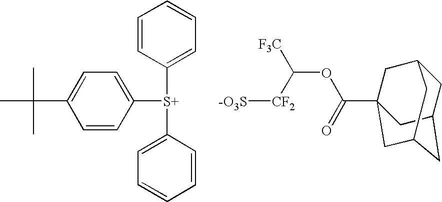

PAG-3

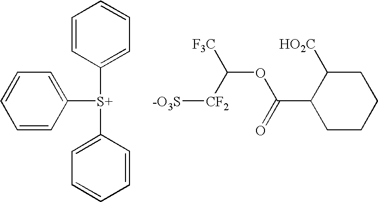

PAG-4

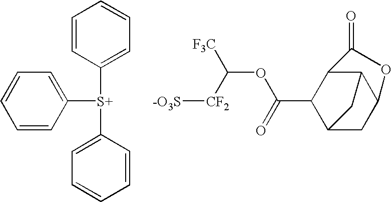

PAG-5

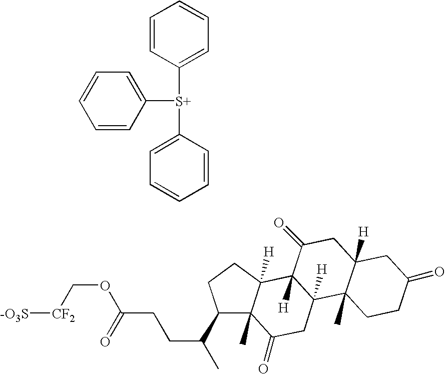

PAG-6

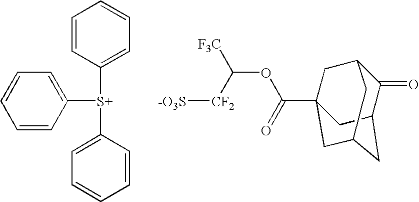

### Resolution Test

### Examples 1 to 30 and Comparative Examples 1 to 4

On silicon wafers having an antireflective coating (ARC29A, Nissan Chemical Industries Ltd.) of 78 nm thick, the resist compositions (R-01 to 30) of the invention and comparative resist compositions (R-31 to 34) were spin coated, then baked at 100° C. for 60 seconds to give resist films having a thickness of 120 nm. Using an ArF excimer laser stepper (Nikon Corp., NA 0.85), the resist films were exposed, baked (PEB) for 60 seconds and then puddle developed for 30 seconds with a 2.38 wt % aqueous solution of tetramethylammonium hydroxide. In this way, 1:1 line-and-space patterns and 1:10 isolated line patterns were formed. The PEB step used an optimum temperature for a particular resist composition.

The pattern-bearing wafers were observed under a top-down scanning electron microscope (TDSEM). The optimum exposure was an exposure dose (mJ/cm²) which provided a 1:1 resolution at the top and bottom of a 80-nm 1:1 line-and-space pattern. The maximum resolution of the resist was defined as the minimum line width (on-mask size, in increments of 5 nm) of a 1:1 line-and-space pattern that was found resolved and separated at the optimum exposure, with smaller values indicating better resolution. The 1:10 isolated line pattern at the optimum exposure was also observed for determining an actual on-wafer size of the isolated line pattern with an on-mask size of 140 nm, which was reported as mask fidelity (on-wafer size, a larger size being better). The pattern profile was visually observed to see whether or not it was rectangular.

Tables 8 and 9 tabulate the test results (maximum resolution, mask fidelity, and profile) of the inventive and comparative resist compositions, respectively.

TABLE 8

| | | | Optimum | Maximum | Mask | |
|---|---|---|---|---|---|---|
| | | PEB temp., | exposure, | resolution, | fidelity, | |
| Example | Resist | ° C. | mJ/cm² | nm | nm | Pattern profile |
| 1 | R-01 | 105 | 37 | 70 | 92 | rectangular |
| 2 | R-02 | 110 | 36 | 70 | 87 | rectangular |
| 3 | R-03 | 115 | 38 | 70 | 92 | rectangular |
| 4 | R-04 | 110 | 38 | 70 | 96 | rectangular |
| 5 | R-05 | 100 | 35 | 70 | 88 | rectangular |
| 6 | R-06 | 115 | 38 | 70 | 92 | rectangular |
| 7 | R-07 | 100 | 34 | 75 | 84 | somewhat rounded top |
| 8 | R-08 | 105 | 45 | 70 | 97 | rectangular |
| 9 | R-09 | 95 | 40 | 70 | 89 | rectangular |
| 10 | R-10 | 95 | 37 | 70 | 92 | rectangular |
| 11 | R-11 | 105 | 38 | 70 | 88 | somewhat T-top |
| 12 | R-12 | 95 | 38 | 70 | 90 | rectangular |
| 13 | R-13 | 95 | 37 | 70 | 92 | rectangular |
| 14 | R-14 | 95 | 37 | 70 | 90 | rectangular |
| 15 | R-15 | 105 | 35 | 70 | 88 | rectangular |
| 16 | R-16 | 105 | 36 | 70 | 91 | rectangular |
| 17 | R-17 | 105 | 35 | 70 | 87 | rectangular |
| 18 | R-18 | 105 | 36 | 70 | 93 | rectangular |
| 19 | R-19 | 95 | 37 | 70 | 91 | rectangular |
| 20 | R-20 | 95 | 39 | 70 | 92 | rectangular |
| 21 | R-21 | 105 | 41 | 70 | 95 | rectangular |
| 22 | R-22 | 105 | 39 | 70 | 94 | rectangular |
| 23 | R-23 | 105 | 38 | 70 | 93 | rectangular |
| 24 | R-24 | 115 | 36 | 70 | 93 | rectangular |
| 25 | R-25 | 105 | 38 | 70 | 94 | rectangular |
| 26 | R-26 | 95 | 43 | 70 | 96 | rectangular |
| 27 | R-27 | 95 | 40 | 70 | 96 | rectangular |
| 28 | R-28 | 95 | 40 | 70 | 95 | rectangular |
| 29 | R-29 | 105 | 38 | 70 | 95 | rectangular |
| 30 | R-30 | 95 | 39 | 70 | 94 | rectangular |

TABLE 9

| | | | Optimum | Maximum | Mask | |
|---|---|---|---|---|---|---|
| Comparative | | PEB temp., | exposure, | resolution, | fidelity, | |
| Example | Resist | ° C. | mJ/cm² | nm | nm | Pattern profile |
| 1 | R-31 | 105 | 340 | 75 | 80 | rectangular |
| 2 | R-32 | 105 | 32.0 | 75 | 78 | somewhat rounded top |
| 3 | R-33 | 105 | 32.0 | 75 | 78 | rectangular |
| 4 | R-34 | 95 | 33.0 | 75 | 81 | rectangular |

It is seen from the results of Table 8 that the resist compositions within the scope of the invention exhibit excellent resolution performance, mask fidelity, and satisfactory pattern profiles. In contrast, Table 9 reveals that Comparative Examples 1 to 4, which use prior art resins, are inferior in maximum resolution and mask fidelity. It has been demonstrated that a resist composition which uses a polymer comprising specific recurring units as a base resin is improved in resolution performance, as compared with resist compositions of the prior art design.

Japanese Patent Application No. 2008-227754 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. A positive resist composition comprising (A) a resin component which becomes soluble in an alkaline developer under the action of an acid and (B) a compound capable of generating an acid in response to actinic light or radiation, wherein
said resin component (A) is a polymer comprising recurring units containing a non-leaving hydroxyl group, represented by the general formula (1): 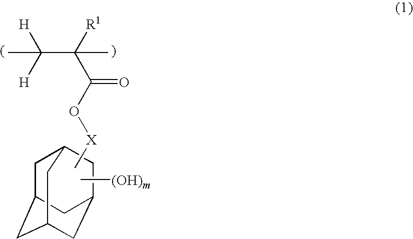
wherein R¹ is hydrogen, methyl or trifluoromethyl, X is a single bond or methylene, and m is 1 or 2, with the proviso that the number “m” of hydroxyl groups each attach to a secondary carbon atom.

2. The composition of claim 1 wherein the polymer as resin component (A) further comprises recurring units of the general formulae (2) and (3): 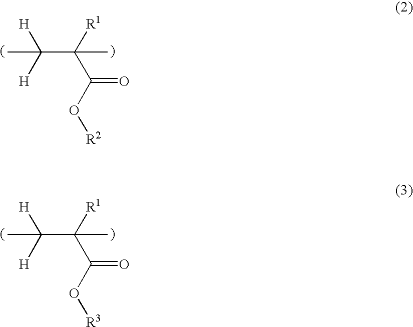
wherein R¹ is each independently hydrogen, methyl or trifluoromethyl, R² is an acid labile group, and R³ is a group containing a 5- or 6-membered lactone ring as a partial structure.

3. The composition of claim 1 wherein said compound (B) is a sulfonium salt compound having the general formula (4): 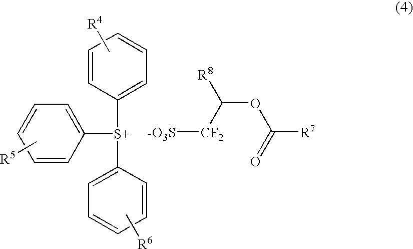
wherein R⁴, R⁵ and R⁶ are each independently hydrogen or a straight, branched or cyclic, monovalent hydrocarbon group of 1 to 20 carbon atoms which may contain a heteroatom, R⁷ is a straight, branched or cyclic, monovalent hydrocarbon group of 7 to 30 carbon atoms which may contain a heteroatom, and R⁸ is hydrogen or trifluoromethyl.

4. A process for forming a pattern, comprising the steps of applying the positive resist composition of claim 1 onto a substrate to form a resist coating, heat treating, exposing the resist coating to high-energy radiation or electron beam through a photomask, heat treating, and developing the exposed coating with a developer.

5. A process for forming a pattern, comprising the steps of applying the positive resist composition of claim 1 onto a substrate to form a resist coating, heat treating, exposing the resist coating to high-energy radiation or electron beam through a photomask, heat treating, and developing the exposed coating with a developer,
the exposing step being effected by the immersion lithography wherein a high refractive index liquid having a refractive index of at least 1.0 intervenes between the resist coating and a projection lens.

6. A process for forming a pattern, comprising the steps of applying the positive resist composition of claim 1 onto a substrate to form a resist coating, heat treating, exposing the resist coating to high-energy radiation or electron beam through a photomask, heat treating, and developing the exposed coating with a developer,
said process further comprising the step of applying a protective coating on the resist coating,
the exposing step being effected by the immersion lithography wherein a high refractive index liquid having a refractive index of at least 1.0 intervenes between the protective coating and a projection lens.

7. A polymerizable compound containing a non-leaving hydroxyl group, represented by the general formula (1a): 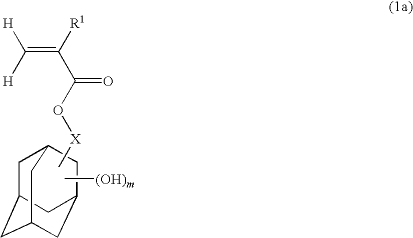
wherein R¹ is hydrogen, methyl or trifluoromethyl, X is a single bond or methylene, and m is 1 or 2, with the proviso that the number “m” of hydroxyl groups each attach to a secondary carbon atom.
